(19) United States
(12) Patent Application Publication
(10) Pub. No.: US 2026/0055158 A1
(43) Pub. Date: Feb. 26, 2026

(54) BIOORTHOGONAL T CELL RECEPTOR MOLECULES AND METHODS OF MAKING AND USING THE SAME

(71) Applicant: The University of North Carolina at Chapel Hill, Chapel Hill, NC (US)

(72) Inventors: Brian Kuhlman, Chapel Hill, NC (US); Tomoaki Kinjo, Chapel Hill, NC (US)

(21) Appl. No.: 19/100,231

(22) PCT Filed: Aug. 8, 2023
(86) PCT No.: PCT/US2023/071864

§ 371 Date: Jan. 31, 2025

Related U.S. Application Data
(60) Provisional application No. 63/370,730, filed on Aug. 8, 2022.

(51) Int. Cl. C07K 14/725; A61K 35/17; A61K 38/00; A61K 40/11; A61K 40/31; A61K 40/32; A61K 40/42; A61K 40/45; A61K 40/46; A61P 35/00; C07K 14/705; C07K 16/00; C12N 5/071; C12N 15/62; C12N 15/86

(52) U.S. Cl. CPC C07K 14/7051; A61K 35/17; A61K 40/11; A61K 40/31; A61K 40/32; A61K 40/42; A61K 40/45; A61K 40/46; A61P 35/00; C07K 14/70596; C07K 16/00; C12N 5/0602; C12N 15/625; C12N 15/86; A61K 38/00; C07K 2319/02; C07K 2319/30; C12N 2510/00

(57) ABSTRACT

This invention relates synthetic T cell receptor molecules and methods of making and using the same.

### STATEMENT OF PRIORITY

This application claims the benefit, under 35 U.S.C. § 119 (e), of U.S. Provisional Application No. 63/370,730, filed Aug. 8, 2022, the entire contents of which is incorporated by reference herein in its entirety.

### STATEMENT OF GOVERNMENT SUPPORT

This invention was made with government support under Grant Number GM131923 awarded by the National Institutes of Health. The government has certain rights in the invention.

### STATEMENT REGARDING ELECTRONIC FILING OF A SEQUENCE LISTING

A Sequence Listing in XML format, entitled 5470-934WO_ST26.xml, 102,558 bytes in size, generated on Aug. 8, 2023, and filed herewith, is hereby incorporated by reference in its entirety for its disclosures.

### FIELD OF THE INVENTION

This invention relates synthetic T cell receptor molecules comprising bioorthogonal interfaces, and methods of making and using the same.

### BACKGROUND OF THE INVENTION

T-cell receptors (TCRs) are attractive agents for cancer treatment because they specifically recognize peptides derived from intracellular proteins, most of which cannot be targeted by small molecules and antibodies. However, the creation of effective TCR drugs is in its infancy because of their inherent weak affinity and cross-reactive properties.

The present invention overcomes previous shortcomings in the art by providing synthetic T cell receptor molecules comprising bioorthogonal interfaces, and methods of making and using the same.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

One aspect of the present invention provides a synthetic T-cell receptor (TCR) molecule comprising: a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen, wherein the TCRCα and TCRCβ or fragments thereof are modified TCRCα and TCRCβ or fragments thereof wherein the first and the second interface are bioorthogonal to each other, and/or wherein the TCRVα and TCRVβ or fragments thereof are modified TCRVα and TCRVβ or fragments thereof wherein the third and the fourth interface are bioorthogonal to each other; wherein the bioorthogonal first and second interface and/or the bioorthogonal third and fourth interface each comprise one or more amino acid substitutions, wherein the bioorthogonal first and second interface selectively bind to each other and/or the bioorthogonal third and fourth interface selectively bind to each other via the one or more substitutions of each interface (e.g., wherein the bioorthogonal TCRC interfaces and/or bioorthogonal TCRV interfaces selectively bind to each other, e.g., selectively bind to each other as compared to unmodified interfaces).

In some embodiments, the synthetic TCR molecule of the present invention comprises the modified TCRCα or fragment thereof with a first interface and the modified TCRCβ or fragment thereof with a second interface which is bioorthogonal to the first interface; and the modified TCRVα or fragment thereof with a third interface, and the modified TCRVβ or fragment thereof with a fourth interface which is bioorthogonal to the third interface.

In some embodiments, the one or more substitutions of the bioorthogonal first and/or second interface comprise paired (e.g., bioorthogonal) knob-into-hole substitutions, charge swap substitutions, charge to hydrophobic substitutions, polar to cation-pi interaction, or any combination thereof.

In some embodiments, the one or more substitutions of the bioorthogonal first interface may comprise substitution at amino acid position 124 (Kabat 122), 126, 126, 128, 130, 132, 133, 134, 138, 140, 142, 144, 145, 158, 161, 163, 168, 171, 172, 173, 175, 177, 179, 181, 183, 205 and/or 207, wherein the numbering corresponds to PDB numbering of the reference TCRCα amino acid sequence of PDB: 6U07_A.

In some embodiments, the one or more substitutions of the bioorthogonal second interface may comprise substitution at amino acid position 126, 128, 130, 131, 133, 135, 136, 139, 140, 142, 144, 146, 148, 170, 175, 177, 179, 181, 182, 184, 185, 193, 170, 195, 197, 202 and/or 204, wherein the numbering corresponds to PDB numbering of the reference TCRCβ amino acid sequence of PDB: 6U07_B.

In some embodiments, the one or more amino acid substitutions of each the bioorthogonal third and fourth interface may be comprised in a conserved region of each of the modified TCRVα or fragment thereof and modified TCRVβ or fragment thereof.

In some embodiments, the one or more substitutions of the bioorthogonal third and/or fourth interface comprise paired (e.g., bioorthogonal) knob-into-hole substitutions, charge swap substitutions, charge to hydrophobic substitutions, polar to cation-pi interaction, or any combination thereof.

In some embodiments, the one or more substitutions of the bioorthogonal third interface may comprise substitution at amino acid position 31, 35, 37, 40, 41, 43, 45, 48, 86, 100, 101, 103, 105 and/or 108 (Kabat 109), wherein the numbering corresponds to PDB numbering of the reference TCRVα amino acid sequence of PDB: 2F53_D.

In some embodiments, the one or more substitutions of the bioorthogonal fourth interface may comprise substitution at amino acid position 7, 8, 9, 29, 31, 33, 35 (Kabat 37), 38 (Kabat 40), 39, 41, 42, 43, 86, 99, 101, 102, 104, 107 and/or 150, wherein the numbering corresponds to PDB numbering of the reference TCR VB amino acid sequence of PDB: 2F53_E.

Also provided herein is the synthetic TCR molecule of the present invention, in soluble form (e.g., wherein the synthetic TCR molecule is devoid of a transmembrane domain).

In some embodiments, the synthetic TCR molecule of the present invention may further comprise a second TCRCα, TCRCβ, TCRVα, and TCRVβ or fragments thereof, each comprising an N-terminus and a C-terminus and a first, second, third and fourth interface, respectively, wherein at least a portion of each of the second TCRVα and TCRVβ together form a variable portion with binding specificity to a second target antigen that is different from the first target antigen.

Also provided herein is the synthetic TCR molecule of the present invention, in soluble form (e.g., wherein the synthetic TCR molecule is devoid of a transmembrane domain).

Also provided herein is the synthetic TCR molecule of the present invention, in cellular form (e.g. further comprising a hinge region (e.g., CD8 hinge, CD4 hinge), a transmembrane domain, a linker, a costimulatory domain (e.g., CD28, 4-1BB, etc.) and/or an scFv and/or Fab, e.g., wherein the synthetic TCR molecule is a chimeric antigen receptor (CAR)).

Another aspect of the present invention provides a nucleic acid molecule (e.g., an isolated nucleic acid molecule) encoding the synthetic TCR molecule of the present invention.

Another aspect of the present invention provides a vector comprising the synthetic TCR molecule of the present invention and/or a nucleic acid molecule of the present invention.

Another aspect of the present invention provides an isolated cell comprising a synthetic TCR molecule of the present invention, nucleic acid molecule, composition and/or vector of the present invention.

Another aspect of the present invention provides a composition comprising the synthetic TCR molecule, nucleic acid molecule, vector, and/or isolated cell of the present invention.

Another aspect of the present invention provides a method of expressing a synthetic TCR molecule in a cell, comprising contacting the cell with a nucleic acid molecule, vector, and/or composition of the present invention.

Another aspect of the present invention provides a method of treating a disorder in a subject, comprising administering to the subject an effective amount of a synthetic TCR molecule, nucleic acid molecule, vector, isolated cell, and/or composition of the present invention, wherein the synthetic TCR binds an antigen associated with the disorder (e.g., a cancer antigen, a viral antigen, a bacterial antigen, or any combination thereof).

Another aspect of the present invention provides a method of producing a synthetic T-cell receptor (TCR) molecule, comprising: (a) providing a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen; (b) introducing one or more amino acid substitutions into the first and second interface and/or into the third and fourth interface, wherein the one or more amino acid substitutions modify the first and the second interface and/or the third and fourth interface such that the interfaces are bioorthogonal to each other (e.g., such that the bioorthogonal interfaces selectively bind to each other, e.g., selectively bind to each other as compared to unmodified interfaces); thereby producing a synthetic TCR molecule (e.g., a synthetic TCR molecule of the present invention).

Another aspect of the present invention provides a method of enhancing stability (e.g., in vitro and/or in vivo) of a T-cell receptor (TCR) molecule, comprising: (a) providing a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen; (b) introducing one or more amino acid substitutions into the first and second interface and/or into the third and fourth interface, wherein the one or more amino acid substitutions modify the first and the second interface and/or the third and fourth interface such that the interfaces are bioorthogonal to each other (e.g., such that the bioorthogonal interfaces selectively bind to each other, e.g., selectively bind to each other as compared to unmodified interfaces), thereby enhancing the stability of the synthetic TCR molecule; thereby producing a synthetic TCR molecule (e.g., a synthetic TCR molecule of the present invention), wherein the synthetic TCR molecule has enhanced stability (e.g., as compared to an unmodified TCR).

### BRIEF DESCRIPTION OF THE DRAWINGS

FIG. 1 shows a schematic of current treatment methods for cancer.

FIG. 2 shows schematics of example embodiments of the invention. FIG. 2 panel A shows an example design of orthogonal TCRs to prevent and/or reduce TCR subunit mispairing. FIG. 2 panel B shows schematics of example designs of soluble bispecific TCRs (left) and cellular bispecific TCR (e.g., CAR-T) therapeutic designs (right).

FIG. 3 shows schematics and data of example TCR structures. FIG. 3 panel A shows a schematic of the TCR-V (variable) and TCR-C (constant) domains of the alpha (α) chain (TCRα) and beta (β) chain (TCRβ). FIG. 3 panel B shows an image of an SDS-PAGE gel of the indicated designs from human and mouse TCRs. FIG. 3 panel C shows a data graph tabulating the melting temperature (Tm in ° C.) of the indicated designs.

FIG. 4 panel A shows a schematic of a validation scheme for the contemplated designs. FIG. 4 panel B shows a data plot of representative data of interface scoring.

FIG. 5 panel A shows schematics of site saturation mutagenesis (SSM) and second site suppressor (SSR) strategies in the development of bio-orthogonal TCR interfaces. FIG. 5 panel B shows representative Rosetta output data of calculated interface scores.

FIG. 6 shows Rosetta models (FIG. 6 panel A) bar graphs (FIG. 6 panels B-E) and data plots (FIG. 6 panel F) related to designed TCRs. (FIG. 6 panel A) Rosetta models for TCR-C (FIG. 6 panel B) nanoDSF and SDS-PAGE of TCR-C designs (FIG. 6 panel C) Rosetta models for TCR-V (FIG. 6 panel E) nanoDSF and SDS-PAGE of TCR-C/V combinative design (FIG. 6 panel F) SPR analysis of WT and designed TCR.

FIG. 7 shows schematics of example soluble forms of the invention. (FIG. 7 panel A) Tandem bsTCR, (FIG. 7 panel B) IgG bsTCR, and (FIG. 7 panel C) bsTCR-based T-cell engager.

FIG. 8 shows schematics of example cellular forms of the invention. (FIG. 8 panel A) Avidity-controlled bsTCR-CAR. (FIG. 8 panel B) bsTCR-CAR with split signaling domains.

FIG. 9 shows results from (FIG. 9 top panel A) nanoDSF and (FIG. 9 bottom panel B) SDS-PAGE of example TCR constant domain designs. The designed TCR α and β subunits were co-expressed in Expi293 cells in single wells as MT/MT, MT/WT, and WT/MT states, and purified by Ni-affinity chromatography. To compare the expression of each state, SDS-PAGE of equal amounts of protein lysate from the different states was performed. To assess the stability of the α/β interface, thermodynamic properties were measured by nano-differential scanning fluorimetry (nanoDSF).

FIG. 10 shows nanoDSF (FIG. 10 top panel A) and SDS-PAGE (FIG. 10 bottom panel B) of example TCR variant domain design. The designed TCR α and β subunits were co-expressed in Expi293 cells in single wells as MT/MT, MT/WT, and WT/MT states, and purified by Ni-affinity chromatography. To compare the expression of each state, SDS-PAGE of equal amounts of protein lysate from the different states was performed. To assess the stability of the α/β interface, thermodynamic properties were measured by nano-differential scanning fluorimetry (nanoDSF).

FIG. 11 shows (FIG. 11 panel A) nanoDSF and (FIG. 11 panel B) SDS-PAGE of TCR-C/V combinative design, and (FIG. 11 panel C) SPR analysis of WT and designed TCR. The designed TCR α and β subunits were co-expressed in Expi293 cells in single wells as MT/MT, MT/WT, and WT/MT states, and purified by Ni-affinity chromatography. To compare the expression of each state, SDS-PAGE of equal amounts of protein lysate from the different states was performed. To assess the stability of the α/β interface, thermodynamic properties were measured by nano-differential scanning fluorimetry (nanoDSF). The binding affinity was measured by Surface plasmon resonance (SPR) analysis.

FIG. 12 shows (FIG. 12 panel A) schematics of Tandem-bsTCR Format. To assemble the Tandem bispecific TCR, the following domains were expressed in Expi293 cells for correct domain assembly: Format-v1) 1) TCR1β-TCR2β, 2) TCR1α, 3) TCR2α, Format-v2: 1) TCR1α-TCR2α, 2) TCR1β 3) TCR2β, Format-v3: 1) TCR1α-TCR2α, 2) TCR1β-TCR2β, and purified by Ni-affinity chromatography. FIG. 12 panel B shows that to compare the expression of each state, SDS-PAGE of equal amounts of protein lysate was performed. The binding affinity was measured by Surface plasmon resonance (SPR) analysis as shown in FIG. 12 panel C.

FIG. 13 shows (FIG. 13 panel A) schematics of IgG bsTCR Format. To assemble the IgG bispecific TCR, the following domains were expressed in Expi293 cells for correct domain assembly: Format-v1) 1) TCR1β-Fc1, 2) TCR2β-Fc2, 3) TCR1α, 4) TCR2α, Format-v2: 1) TCR1α-Fc1, 2) TCR2α-Fc2, 3) TCR1β, 4) TCR2β, and purified by Ni-affinity chromatography. To compare the expression of each state, SDS-PAGE (FIG. 13 panel B) of equal amounts of protein lysate was performed. The binding affinity was measured by Surface plasmon resonance (SPR) analysis (FIG. 13 panel C).

FIGS. 14A-14D show schematics of the TCRCα PDB: 6U07_A (FIG. 14A; SEQ ID NO: 1), TCRCβ PDB: 6U07_B (FIG. 14B; SEQ ID NO:2), TCRVα PDB: 2F53_D (FIG. 14C; SEQ ID NO:31) and TCRVβ PDB: 2F53_E (FIG. 14D; SEQ ID NO:32) reference amino acid TCR sequences used for numbering. Figures are related to Tables 6 to 9 providing alignments between commonly used numbering systems in the art for antibody and TCR structures including PDB, Kabat, and IMGT.

FIG. 15 shows an alignment of example non-limiting generated TCRCα, TCRCβ, TCRVα, and TCRVβ designs of the invention. Sequences shown correspond to SEQ ID NOs: 1, 3, 5, 7, 9, 11, 13, 15, 17, 19, 21, 23, 25, 27 and 29 (TCRCa_Design_alignment); SEQ ID NOs: 2, 4, 6, 8, 10, 12, 14, 16, 18 and 20 (TCRCb_Design_alignment); SEQ ID NO:31, 33, 35, 37, 39, 41, 43 and 45 (TCRVa_Design_alignment); SEQ ID NOs: 32, 34, 36, 38, 40, 42, 44 and 46 (TCRVb_Design_alignment); SEQ ID NO:31 as compared to 1G4-122 (desV30combiC46; SEQ ID NO:33) and 1G4-c49c50 (SEQ ID NO:49) (TCRVa_variation); and SEQ ID NO:32 as compared to 1G4-122 (desV30combiC46; SEQ ID NO:34) and 1G4-c49c50 (SEQ ID NO: 50) (TCRVb_variation).

FIG. 16 shows additional example design configurations for trispecific constructs, as related to FIG. 7, referred to therein as trispecific T-cell engager or “TriTE”. The left shows a schematic based on use of a scFv region between the TCR1 and TCR2. The right shows a schematic based on use of a Fab region between the TCR1 and TCR2.

FIG. 17 shows images of SDS-PAGE blots of the TCR constant domain designs as indicated. The designed TCR α and β subunits were coexpressed in Expi293 cells in single wells as MT/MT, MT/WT, and WT/MT states, and purified by Ni-affinity chromatography. To compare the expression of each state, SDS-PAGE of equal amounts of protein lysate from the different states was performed. To assess the stability of the α/β interface, thermodynamic properties were analyzed by nanoDSF. Figure terminology and detailed information of TriTE domains: TCR1a: α chain of 1G4-122 TCR (desV30combiC46); TCR1b: β chain of 1G4-122 TCR (desV30combiC46); TCR2a: α chain of MEL5-α24β17 TCR (wtVstC); TCR2b: β chain of MEL5-α24β17 TCR (wtVstC) LCimc: light chain of anti-CD3 Fab, variant domain with anti-CD3 “imc” clone from ImmTAC and constant domain CA of human IgG; HCimc: heavy chain of anti-CD3 Fab, variant domain with anti-CD3 clone from ImmTAC, constant domain CHI of human IgG. LCsp: light chain of anti-CD3 Fab, variant domain with anti-CD3 clone “SP34” and constant domain Cλ of human IgG; HCsp: heavy chain of anti-CD3 Fab, variant domain, with anti-CD3 clone SP34, constant domain CHI of human IgG.

FIG. 18 shows images of data plots related to tumor killing assays. A375 melanoma cells were pulsed with NY-ESO-1 peptide and MART-1 peptide, and then exposed to TriTE constructs 1 through 24.

FIG. 19 shows an image of an example data plot related to tumor killing assays pulsed with different peptides as indicated, and exposed to TriTE19.

FIG. 20 shows images of data plots related to tumor killing assays. SK-MEL5 cells expressing GFP-luciferase labeled SK-MEL-5 endogenous peptide were exposed to TriTE constructs 1 through 24.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

As used herein, the term “antigen” refers to a molecule capable of inducing the production of immunoglobulins (e.g., antibodies). As used herein, the term “immunogen” refers to when a molecule is capable of inducing a multi-faceted humoral and/or cellular-mediated immune response. In some embodiments, an antigen may be referred to as an immunogen, e.g., under conditions when the antigen is capable of inducing a multi-faceted humoral and/or cellular-mediated immune response. A molecule and/or composition (e.g., including but not limited to a nucleic acid, protein, polysaccharide, ribonucleoprotein (RNP), whole bacterium, and/or composition comprising the same) that is capable of antibody may be referred to as “antigenic” and/or that is capable of immune response stimulation may be referred to as “immunogenic,” and can be said to have the ability of antigenicity and/or immunogenicity, respectively. The binding site for an antibody within an antigen and/or immunogen may be referred to as an epitope (e.g., an antigenic epitope).

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, or can be a chimeric or humanized antibody. See, e.g., Walker et al., Molec. Immunol. 26:403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980. The antibody can further be a single chain antibody or bispecific antibody. The antibody can also be humanized for administration to a human subject.

Non-limiting examples of an antibody or fragment thereof of the present invention include a monoclonal antibody or fragment thereof, a chimeric antibody or fragment thereof, a CDR-grafted antibody or fragment thereof, a humanized antibody or fragment thereof, an Fc, a Fab, a Fab′, a F(ab′)2, a Fv, a disulfide linked Fv, a single chain antibody (scFv), a single domain antibody (dAb), a diabody, a multispecific antibody (e.g., a bispecific antibody) or fragment thereof, an anti-idiotypic antibody or fragment thereof, a bifunctional hybrid antibody or fragment thereof, a functionally active epitope-binding antibody fragment, an affibody, a nanobody, and any combination thereof. Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′) 2 fragments. Alternatively, Fab expression libraries can be constructed to allow rapid and easy identification of monoclonal Fab fragments with the desired specificity (Huse et al., (1989) Science 254:1275-1281).

As used herein “T cell receptor” and/or “TCR” refers to natural, modified, and/or synthetic protein structures related to native TCR of a T cell or NKT cell as known in the art. Native TCRs are transmembrane receptors expressed on the surface of T and/or NKT cells that recognize antigens bound to major histocompatibility complex molecules (MHC). Native TCRs are heterodimeric and comprise an alpha (α) chain comprising a constant domain (TCRCα) and a variable domain (TCRVα), and a beta (β) chain comprising a constant domain (TCRCβ) and a variable domain (TCRVβ), wherein the alpha and beta chains are linked through a disulfide bond. The TCR is expressed as part of a complex with accessory proteins which include CD3 (e.g., CD3 epsilon (ε), zeta (ζ), delta (δ)). TCR structures are known in the art, as will be readily apparent to the skilled artisan, and are further described for example in Janeway's Immunobiology, 10^{th} edition, Murphy et al. 2022.

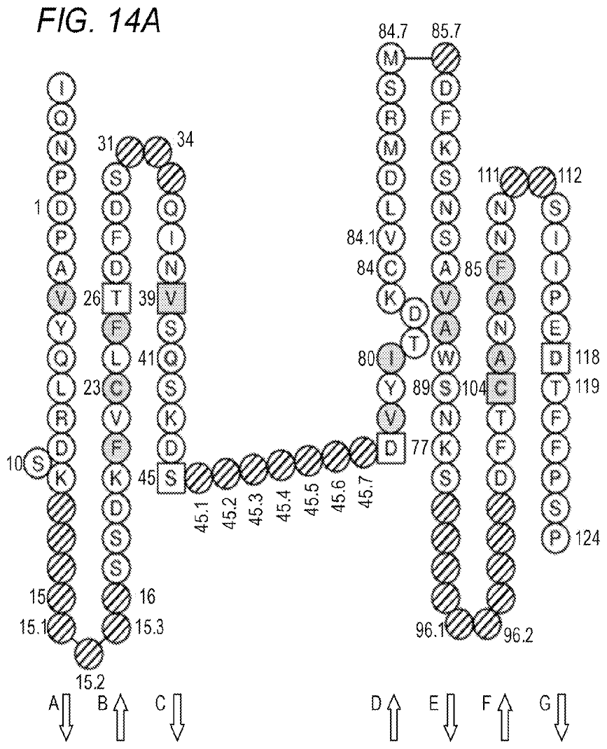

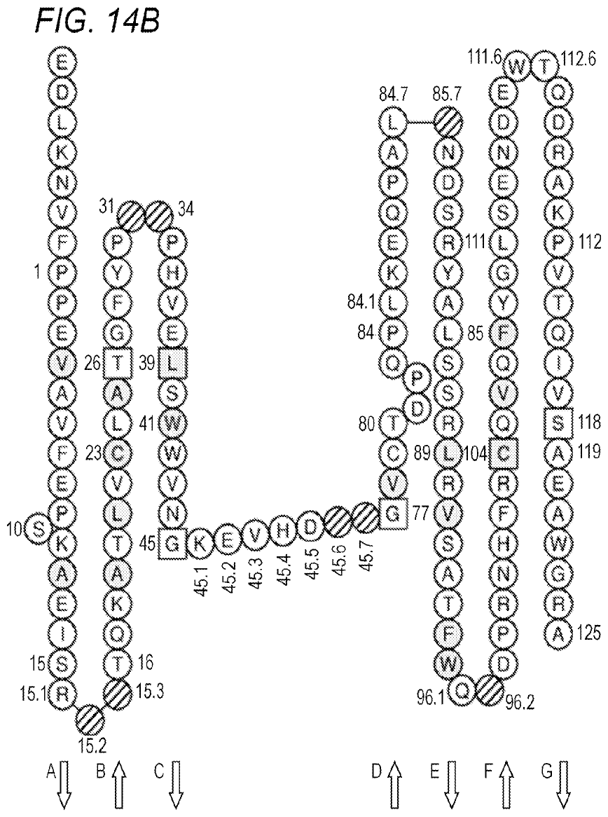

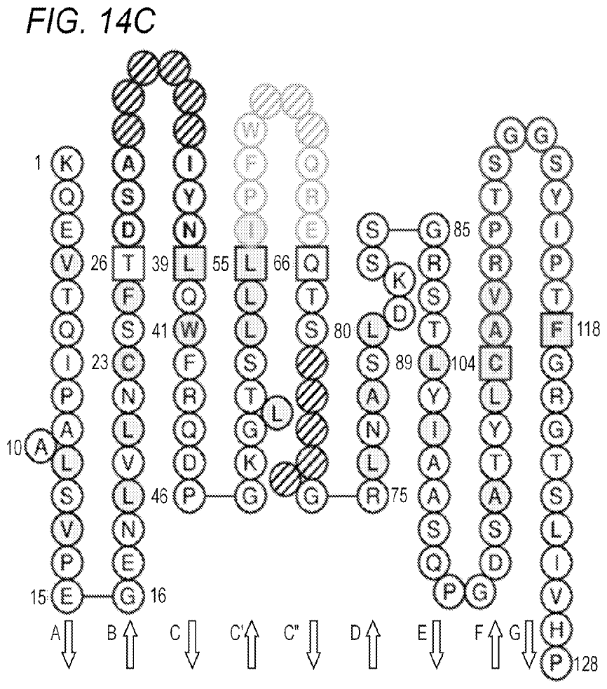

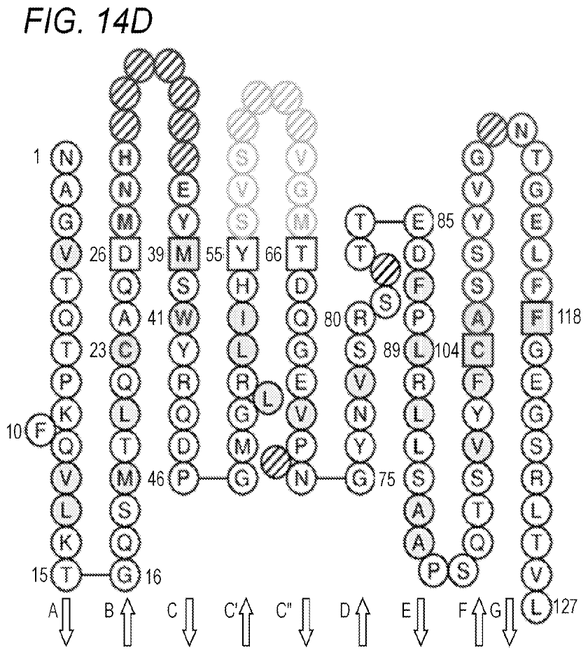

The methods and compounds of the present invention comprise designed amino acid modifications at particular residues within the variable and constant domains of TCR alpha and/or beta chains. As one of ordinary skill in the art will appreciate, various numbering conventions may be employed for designating particular amino acid residues within TCR alpha and beta chains. Commonly used conventions that may include corrections or alternate numbering systems for variable domains include the designations of sequences and structures as found in the Protein Data Bank (PDB; Berman et al. 2000 Nucleic Acids Research 28 (1): 235-242; see rcsb.org/); Kabat (see, Kabat et al., Sequences of Proteins of Immunological Interest, 5th Ed, Public Health Service, National Institutes of Health, Bethesda, Md. (1991)), Chothia (Chothia C, Lesk A M (1987), J Mol Biol 196:901-917; Chothia, et al. (1989), Nature 342:877-883), IMGT (Lefranc, et al. (2003), Dev Comp Immunol 27:55-77), and AHo (Honegger A, Pluckthun A (2001) J Mol Biol 309:657-670). These references provide amino acid sequence numbering schemes for TCR alpha and beta chains that define the location of amino acid residues of antibody and TCR sequences based on structure similarity. Unless otherwise expressly stated herein, all references to amino acid residues (i.e. numbers and/or positions) appearing in the Examples and Claims are based on the numbering associated with the reference TCRCα, TCRCβ, TCRVα and/or TCRVβ deposit sequences as found in the Protein Data Bank for reference TCRs 6U07 and 2F53, the disclosures of which are incorporated herein by reference. With knowledge of the residue number according to PDB, Kabat or IMGT numbering, one of ordinary skill can apply the teachings of the art to identify amino acid sequence modifications within the present invention, according to any commonly used numbering convention. While the Examples and Claims of the present invention employ PDB and/or Kabat numbering to identify particular amino acid residues, it is understood that SEQ IDs appearing in a Sequence Listing accompanying the present application provide sequential numbering of amino acids within a given polypeptide and, thus, do not conform to the corresponding amino acid numbers as provided by the PDB and/or Kabat numbering convention. Alignments between the PDB assigned numbering, Kabat, and IMGT are provided in Tables 6-9 and FIGS. 14A-14D.

“Effective amount” as used herein refers to an amount of a vector, nucleic acid molecule, epitope, polypeptide, cell, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The terms “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing a disorder by the methods of the present invention can be determined by detecting a clinical improvement as indicated by a change in the subject's symptoms and/or clinical parameters (e.g., viremia for a viral infection, etc.), as would be well known to one of skill in the art.

Unless indicated otherwise, the terms “protect,” “protecting,” “protection” and “protective” (and grammatical variations thereof) encompass both methods of preventing and treating a disorder in a subject.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “subject” of the invention includes any animal susceptible to a disorder expressing and/or associated with an antigen to which a synthetic TCR of the present invention binds (e.g., a cancer antigen, a viral antigen, a bacterial antigen, or any combination thereof). Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. In some embodiments, a laboratory animal may include but is not limited to any standard laboratory mouse strain.

A “subject in need” of the methods of the invention can be a subject known to be, or suspected of being, infected with, or at risk of being infected with, a disorder and/or infection comprising an antigen targeted by a synthetic TCR of the present invention (e.g., wherein the TCR of the present invention comprises a variable portion with binding specificity to the antigen expressed and/or associated with the disorder).

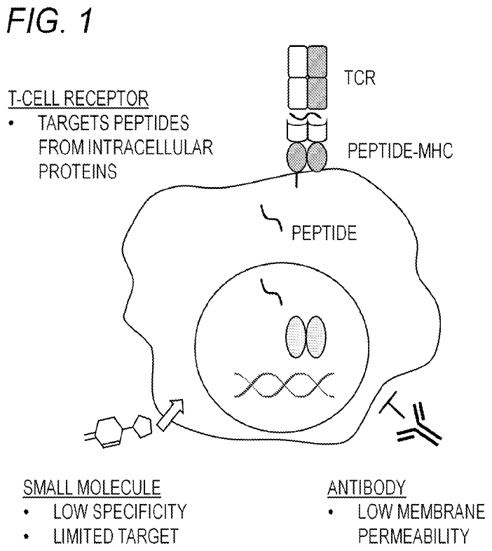

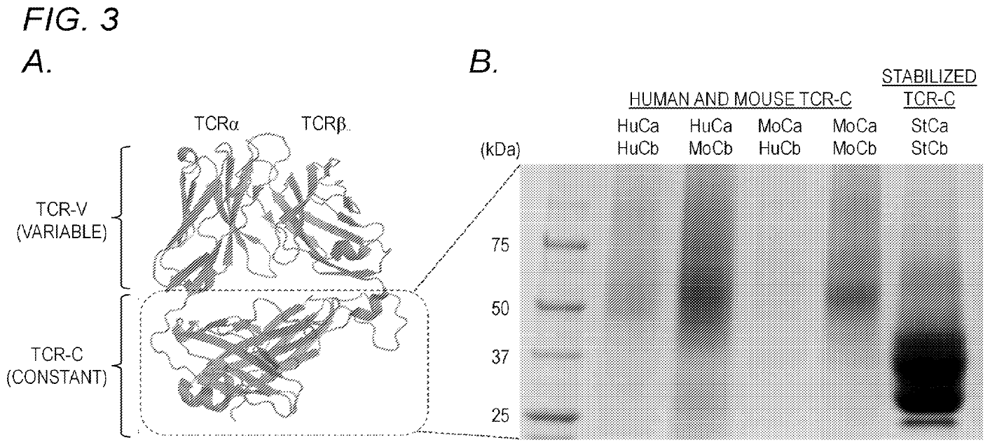

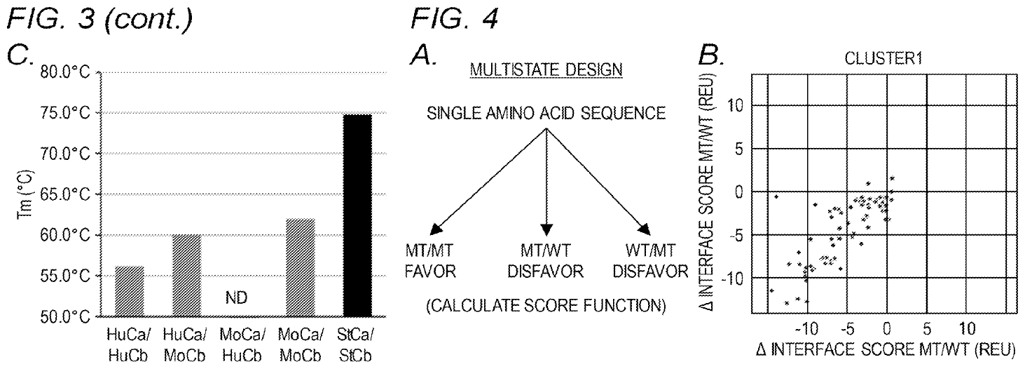

A challenge in cancer treatment is precise recognition of cancer cells because cancer cells rarely possess a single surface marker that distinguishes them from healthy cells. Most intracellular proteins that drive cancerous transformation are difficult to attack with small molecules because the proteins have large flat surfaces and lack deep hydrophobic pockets. They are also difficult targets for antibodies due to membrane impermeability of antibody (FIG. 1). T-cell receptors (TCRs) are attractive in the field of cancer therapeutics because TCRs recognize unique peptides that are derived from intracellular proteins and displayed on the cell surface by major histocompatibility complexes. Despite these properties, TCRs inherently have weak affinity and cross-reactive properties that significantly prevent the development of TCR-based therapeutics. For example, TCRs are composed of an α and β chain, each containing a variable (TCR-V) and constant (TCR-C) domain (as shown in a representative model in FIG. 3 panel A) that structurally resemble antibodies, but unlike antibodies, TCRs are inherently unstable and tend to aggregate, which has posed a technical challenge in attempts to develop TCR-based therapeutics. While not wishing to be bound to theory, effective TCR therapeutics may require simultaneous targeting of two different molecules to increase total avidity and specificity to their target, e.g., cancer cells.

A major technical challenge in engineering bispecific TCRs is subunit mispairing. A TCR is composed of an α and β chain, and co-expression of two different TCRs produces misassembled by-products that cause undesired molecular specificities and prevent the efficient generation of TCR therapeutics (as described in references 16 and 17, incorporated herein by reference). Stabilized TCRs and TCR therapeutics have been generated, including ImmTAC (single TCR-based bispecific T-cell redirector), such as described in Liddy et al. 2012 Nature Medicine 18:980-987; Froning et al. 2020 Nature Communications 11:2330; Nathan et al. 2021 NJEM 385:1196-1206; drugs.ncats.io/drug/N658GY6L3E; and U.S. Pat. No. 9,068,178, the disclosures of each of which are incorporated herein by reference. Strategies in the field for designing transgenic TCRs without mispairing to endogenous TCRs include adding interchain disulfide bonds, using single-stranded TCRs, and exchanging constant domains with murinized portions. However, none of these strategies sufficiently avoid mispairing.

Computer-based approaches have been developed for modeling stabilized proteins and creating new structures with predefined conformations. Rosetta is a software suite for macromolecular design which relies on the rotamer-based sampling of amino acid side chains and an energy function that accounts for atomic interactions, packing, and implicit solvation (references 18 and 19). Rosetta has been used in structure prediction, engineering of antibody binding sites, de novo design of proteins, and engineering of bispecific antibodies via the design of orthogonal interfaces between Fc homodimer as well as heavy chain and light chain interface of antibodies, such as described in references 22 and 23 and U.S. Pat. No. 10,047,167, the disclosures of each of which are incorporated herein by reference.

The present invention is based on the strategy of improving synthetic TCR stability, avidity, and specificity through designing TCRs without and/or reduced mispairing to endogenous TCRs by introducing paired bioorthogonal modifications into the interfaces of corresponding alpha (α) and beta (β) chains of the synthetic TCRs.

One aspect of the present invention provides synthetic T-cell receptor (TCR) molecule comprising: a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen, wherein the TCRCα and TCRCβ or fragments thereof are modified TCRCα and TCRCβ or fragments thereof wherein the first and the second interface are bioorthogonal to each other, and/or wherein the TCRVα and TCRVβ or fragments thereof are modified TCRVα and TCRVβ or fragments thereof wherein the third and the fourth interface are bioorthogonal to each other; wherein the bioorthogonal first and second interface and/or the bioorthogonal third and fourth interface each comprise one or more amino acid substitutions, wherein the bioorthogonal first and second interface selectively bind to each other and/or the bioorthogonal third and fourth interface selectively bind to each other via the one or more substitutions of each interface (e.g., wherein the bioorthogonal TCRC interfaces and/or bioorthogonal TCRV interfaces selectively bind to each other, e.g., selectively bind to each other as compared to unmodified interfaces).

As used herein, the terms “TCR alpha chain constant domain (TCRCα),” “TCR beta chain constant domain (TCRCβ),” “TCR alpha chain variable domain (TCRVα),” and/or “TCR beta chain variable domain (TCRVβ)” refer to known domain components of a standard TCR molecule as understood in the field, as will be apparent to one skilled in the art upon review of the present disclosure.

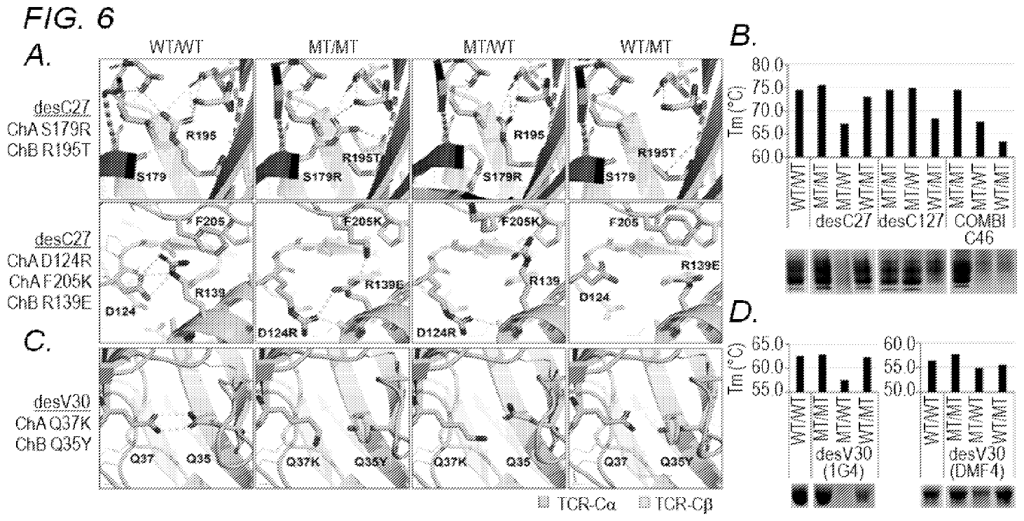

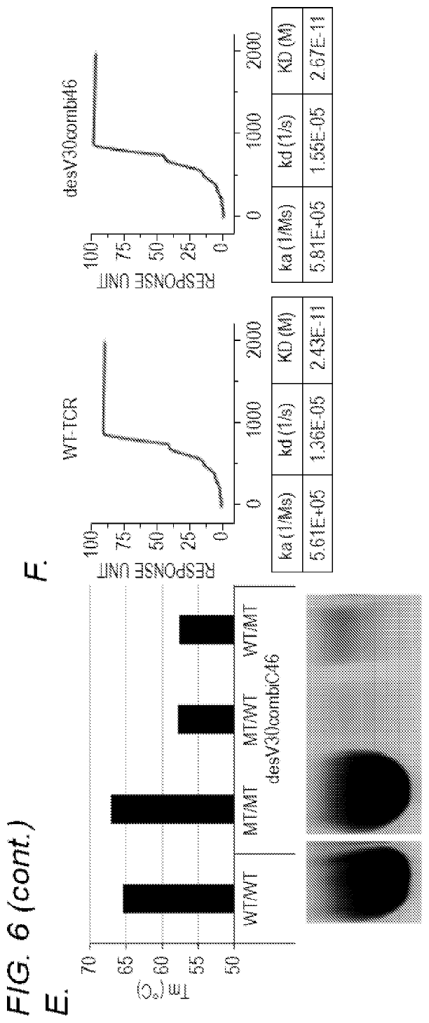

As used herein, the term “interface” refers to the sites of interaction between the alpha and beta chains of the TCR molecule, e.g., the sites of interaction between the alpha chain constant domain and the beta chain constant domain, and/or the sites of interaction between the alpha chain variable domain and the beta chain variable domain. For example, a non-limiting schematic example of such sites is shown in FIG. 6 panel A. However, any residue within the TCRCα, TCRCβ, TCRVα, and/or TCRVβ which may interact with its corresponding alpha or beta chain partner is contemplated as a modification site with the interfaces (e.g., first, second, third, and/or fourth interface) of the present invention.

As used herein, the terms “bioorthogonal” and/or “orthogonal” refer to paired modifications on two interfaces (e.g., the first and second interface and/or the third and fourth interface) which modify the corresponding TCRCα, TCRCβ, TCRVα, and/or TCRVβ such that the TCRCα, TCRCβ, TCRVα, and/or TCRVβ selectively bind to their corresponding pair (e.g., TCRCα and TCRCβ and/or TCRVa, and/or TCRVβ), e.g., wherein the TCRCα and TCRCβ first and second interfaces selectively bind to each other (e.g., have enhanced binding affinity to each other) and/or the TCRVα and TCRVβ third and fourth interfaces selectively bind to each other (e.g., have enhanced binding affinity to each other) as compared to their binding affinity and/or selective binding to an unmodified corresponding pair. Without wishing to be bound to theory, modified TCRCα, TCRCβ, TCRVα, and/or TCRVβ of the present invention comprises bioorthogonal interfaces which enhance pairing and reduce and/or eliminate mispairing with endogenous and/or unmodified TCRCα, TCRCβ, TCRVα, and/or TCRVβ, thereby enhancing total avidity, specificity, and/or stability of the synthetic TCR molecule. In some embodiments, bioorthogonality refers to the ability of the pairing to selectively occur and/or be retained in in vitro and/or in vivo conditions such that the pairing avoids side reactions with other biological compounds (e.g., mispairing with endogenous and/or other TCR alpha and/or beta chains), and/or are non-toxic and functional in appropriate biological conditions.

As used herein, the term “variable portion” refers to the portion of the TCRα and/or TCRβ chain variable domain which comprises a variable region that defines the binding specificity to a target antigen (e.g., the first and/or second target antigen of the present invention). The variable region(s) of a TCR molecule is a known term in the art, and such a region would be readily determinable by one of skill in the art upon review of the present disclosure.

A “target antigen” as used herein refers to a molecule that binds to a variable region of the TCRα and/or TCRβ chain comprised within the variable portion.

In some embodiments, the synthetic TCR molecule of the present invention may comprise the modified TCRCα or fragment thereof with a first interface and the modified TCRCβ or fragment thereof with a second interface which is bioorthogonal to the first interface; and an unmodified TCRVα or fragment thereof with a third interface, and an unmodified TCRVβ or fragment thereof with a fourth interface which is bioorthogonal to the third interface.

In some embodiments, the synthetic TCR molecule of the present invention may comprise an unmodified TCRCα or fragment thereof with a first interface and an unmodified TCRCβ or fragment thereof with a second interface which is bioorthogonal to the first interface; and the modified TCRVα or fragment thereof with a third interface, and the modified TCRVβ or fragment thereof with a fourth interface which is bioorthogonal to the third interface.

In some embodiments, the synthetic TCR molecule of the present invention may comprise the modified TCRCα or fragment thereof with a first interface and the modified TCRCβ or fragment thereof with a second interface which is bioorthogonal to the first interface; and the modified TCRVα or fragment thereof with a third interface, and the modified TCRVβ or fragment thereof with a fourth interface which is bioorthogonal to the third interface.

In some embodiments, each of the (modified and/or unmodified) TCRCα, TCRCβ, TCRVα, and TCRVα domains or fragment thereof comprises an N-terminus and a C-terminus, and wherein the TCRCα C-terminus is linked to the TCRVα N-terminus, and wherein the TCRCβ C-terminus is linked to the TCRVβ N-terminus.

The synthetic TCR molecule of the present invention may comprise any pair of modifications (e.g., amino acid substitution, insertion and/or deletion) wherein one of the paired modifications is comprised in the first interface and the other of the paired modifications is comprised in the second interface; and/or wherein one of the paired modifications is comprised in the third interface and the other of the paired modifications is comprised in the fourth interface, such that the first and second interfaces and/or third and fourth interfaces are bioorthogonal to each other, e.g., selectively bind to each other over an unmodified corresponding first, second, third or fourth interface.

In some embodiments, the one or more substitutions of the bioorthogonal first interface and/or the second interface comprise paired (e.g., bioorthogonal) knob-into-hole substitutions, charge swap substitutions, charge to hydrophobic substitutions, polar to cation-pi interaction, or any combination thereof.

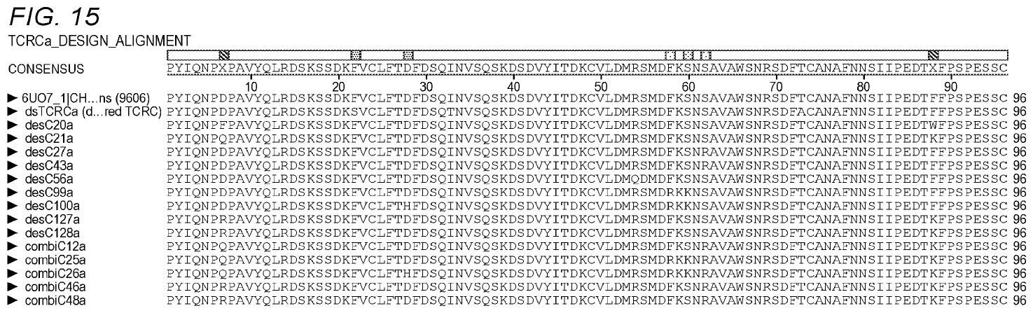

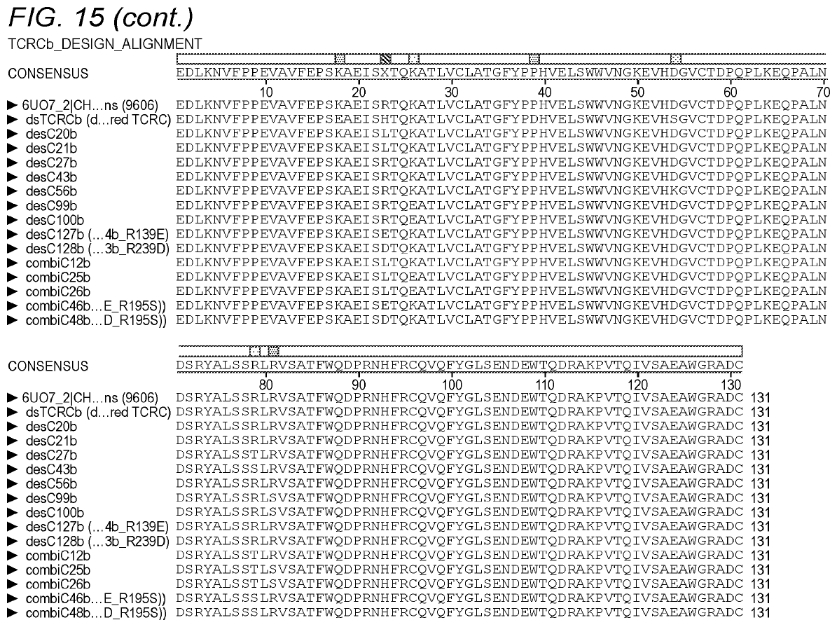

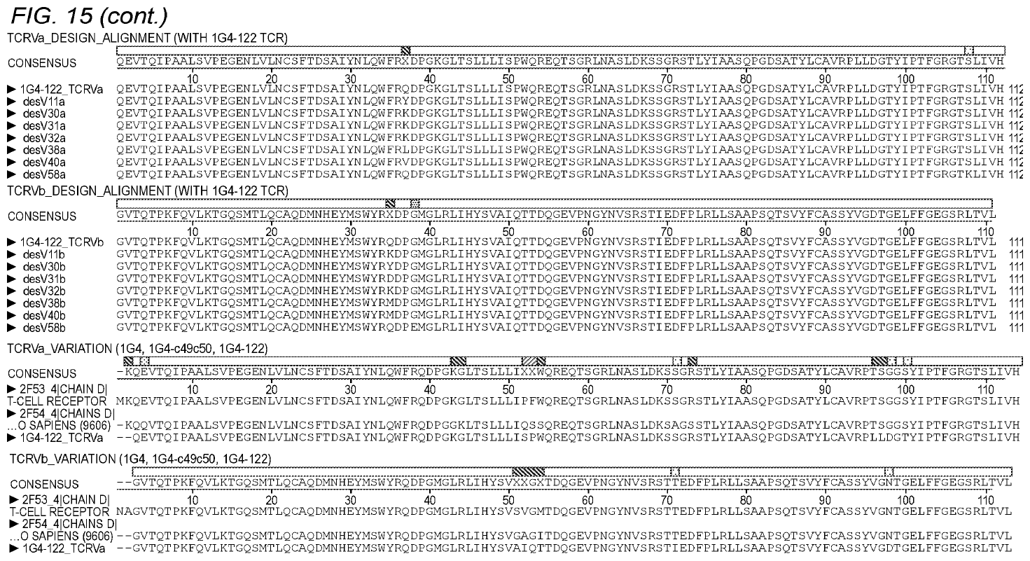

In some embodiments, the one or more substitutions of the bioorthogonal first interface comprise substitution at amino acid position 124 (Kabat 122), 126, 126, 128, 130, 132, 133, 134, 138, 140, 142, 144, 145, 158, 161, 163, 168, 171, 172, 173, 175, 177, 179, 181, 183, 205 and/or 207, wherein the numbering corresponds to PDB numbering of the reference TCRCα amino acid sequence of PDB: 6U07_A. Additional non-limiting examples of the one or more substitutions of the bioorthogonal first interface include any amino acid position and/or residue change as described in FIGS. 14A-14D and 15; and Tables 1-9.

In some embodiments, the one or more substitutions of the bioorthogonal first interface comprise substitution at amino acid position 124 (Kabat 122), 145, 171, 172, 175, 177, 179, and/or 205, wherein the numbering corresponds to PDB numbering of the reference TCRCα amino acid sequence of PDB: 6U07_A (e.g., PDB numbering within residues 118-213 of the reference TCRCα amino acid sequence of 6U07_A (Chain A) in the Protein Data Bank (rcsb.org/sequence/6U07)).

### SEQ ID NO:1 Residues 118-124 of PDB: 6U07 A (TCR Constant Domain of α Chain; TCRCα):

PYIQNPDPAVYQLRDSKSSDKFVCLFTDFDSQINVSQSKDSDVYITDKC
VLDMRSMDFKSNSAVAWSNKSDFTCANAFNNSIIPEDTFFPSPESSC

In some embodiments, the one or more substitutions of the bioorthogonal first interface comprise 124F, 124Q, 124R (Kabat 122F, 122Q, 122R), 145H, 171Q, 172D, 175R, 177K, 179R, and/or 205K.

In some embodiments, the one or more substitutions of the bioorthogonal first interface comprise D124F, D124Q, D124R (Kabat D122F, D122Q, D122R), D145H, R171Q, S172D, F175R, S177K, S179R, and/or F205K.

In some embodiments, the one or more substitutions of the bioorthogonal second interface comprise substitution at amino acid position 126, 128, 130, 131, 133, 135, 136, 139, 140, 142, 144, 146, 148, 170, 175, 177, 179, 181, 182, 184, 185, 193, 170, 195, 197, 202 and/or 204, wherein the numbering corresponds to PDB numbering of the reference TCRCβ amino acid sequence of PDB: 6U07_B. Additional non-limiting examples of the one or more substitutions of the bioorthogonal second interface include any amino acid position and/or residue change as described in FIGS. 14A-14D and 15; and Tables 1-9.

In some embodiments, the one or more substitutions of the bioorthogonal second interface comprise substitution at amino acid position 139, 142, 170, 195 and/or, 197, wherein the numbering corresponds to PDB numbering of the reference TCRCβ amino acid sequence of PDB: 6U07_B (e.g., PDB numbering within residues 117-247 of the reference TCRCβ amino acid sequence of 6U07_B (Chain B) in the Protein Data Bank (rcsb.org/sequence/6U07)).

### SEQ ID NO:2 Residues 117-247 of PDB: 6U07 B (TCR Constant Domain of β Chain; TCRCβ):

EDLKNVFPPEVAVFEPSKAEISRTQKATLVCLATGFYPPHVELSWWVNG
KEVHDGVCTDPQPLKEQPALNDSRYALSSRLRVSATFWQDPRNHFRCQV
QFYGLSENDEWTQDRAKPVTQIVSAEAWGRADC

In some embodiments, the one or more substitutions of the bioorthogonal second interface comprise 139L, 139D, 139E, 142E, 170K, 195T, 195S and/or 197S.

In some embodiments, the one or more substitutions of the bioorthogonal first interface comprise R139L, R139D, R139E, K142E, D170K, R195T, R195S and/or R197S.

In some embodiments, the one or more substitutions of the bioorthogonal first interface and the bioorthogonal second interface comprise 179R in the first interface and 195S in the second interface (e.g., desC43).

In some embodiments, the one or more substitutions of the bioorthogonal first interface and the bioorthogonal second interface comprise 124R and 205K in the first interface and 139E in the second interface (e.g., desC127).

In some embodiments, the one or more substitutions of the bioorthogonal first interface and the bioorthogonal second interface comprise 124R, 179R and 205K in the first interface and 139E and 195S in the second interface (e.g., combiC46).

In some embodiments, the one or more substitutions of the bioorthogonal first interface and the bioorthogonal second interface comprise 124Q and 205K in the first interface and 139L in the second interface (e.g., desC21).

In some embodiments, the one or more substitutions of the bioorthogonal first interface and the bioorthogonal second interface comprise 179R in the first interface and 195T in the second interface (e.g., desC27).

In some embodiments, the one or more substitutions of the bioorthogonal first interface and the bioorthogonal second interface comprise 175R and 177K in the first interface and 142E and 197S in the second interface (e.g., desC99).

In some embodiments, the one or more substitutions of the bioorthogonal first interface and the bioorthogonal second interface comprise 124F and 205W in the first interface and 139L in the second interface (e.g., desC20).

In some embodiments, the one or more substitutions of the bioorthogonal first interface and the bioorthogonal second interface comprise 171Q and 172D in the first interface and 170K in the second interface (e.g., desC56).

In some embodiments, the one or more substitutions of the bioorthogonal first interface and the bioorthogonal second interface comprise 145H, 175R and 177K in the first interface and 142E and 197S in the second interface (e.g., desC100).

In some embodiments, the one or more substitutions of the bioorthogonal first interface and the bioorthogonal second interface comprise 124R and 205K in the first interface and 139D in the second interface (e.g., desC128).

In some embodiments, the one or more substitutions of the bioorthogonal first interface and the bioorthogonal second interface comprise 124Q, 179R, and 205K in the first interface and 139L and 195T in the second interface (e.g., combiC12).

In some embodiments, the one or more substitutions of the bioorthogonal first interface and the bioorthogonal second interface comprise 124Q, 145H, 185R, 177K, 179R and 205K in the first interface and 139L, 142E, 195T and 197S in the second interface (e.g., combiC26).

In some embodiments, the one or more substitutions of the bioorthogonal first interface and the bioorthogonal second interface comprise 124R, 179R and 205K in the first interface and 139D and 195S in the second interface (e.g., combiC48).

In some embodiments, the one or more substitutions of the bioorthogonal first interface and the bioorthogonal second interface comprise 124Q, 175R, 177K, 179R, and 205K in the first interface and 139L, 142E, 195T and 197S in the second interface (e.g., combiC25).

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC43):

(TCRCα; SEQ ID NO: 3)
PYIQNPDPAVYQLRDSKSSDKFVCLFTDFDSQINVSQSKDSDVYITDKC
VLDMRSMDFKSNRAVAWSNKSDFTCANAFNNSIIPEDTFFPSPESSC,
and
(TCRCβ; SEQ ID NO: 4)
EDLKNVFPPEVAVFEPSKAEISRTQKATLVCLATGFYPPHVELSWWVN
GKEVHDGVCTDPQPLKEQPALNDSRYALSSSLRVSATFWQDPRNHFRC
QVQFYGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC127):

(TCRCα;)
SEQ ID NO: 5
PYIQNPRPAVYQLRDSKSSDKFVCLFTDFDSQINVSQSKDSDVYITDKCV
LDMRSMDFKSNSAVAWSNKSDFTCANAFNNSIIPEDTKFPSPESSC,
and
(TCRCβ;)
SEQ ID NO: 6
EDLKNVFPPEVAVFEPSKAEISETQKATLVCLATGFYPPHVELSWWVNGK
EVHDGVCTDPQPLKEQPALNDSRYALSSRLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (combiC46):

(TCRCα;)
SEQ ID NO: 7
PYIQNPRPAVYQLRDSKSSDKFVCLFTDFDSQINVSQSKDSDVYITDKCV
LDMRSMDFKSNRAVAWSNKSDFTCANAFNNSIIPEDTKFPSPESSC,
and
(TCRCβ;)
SEQ ID NO: 8
EDLKNVFPPEVAVFEPSKAEISETQKATLVCLATGFYPPHVELSWWVNGK
EVHDGVCTDPQPLKEQPALNDSRYALSSSLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC21):

(TCRCα;)
SEQ ID NO: 9
PYIQNPQPAVYQLRDSKSSDKFVCLFTDFDSQINVSQSKDSDVYITDKCV
LDMRSMDFKSNSAVAWSNKSDFTCANAFNNSIIPEDTKFPSPESSC,
and
(TCRCβ;)
SEQ ID NO: 10
EDLKNVFPPEVAVFEPSKAEISLTQKATLVCLATGFYPPHVELSWWVNGK
EVHDGVCTDPQPLKEQPALNDSRYALSSRLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC27):

(TCRCα;)
SEQ ID NO: 11
PYIQNPDPAVYQLRDSKSSDKFVCLFTDFDSQINVSQSKDSDVYITDKCV
LDMRSMDFKSNRAVAWSNKSDFTCANAFNNSIIPEDTFFPSPESSC,
and
(TCRCβ;)
SEQ ID NO: 12
EDLKNVFPPEVAVFEPSKAEISRTQKATLVCLATGFYPPHVELSWWVNGK
EVHDGVCTDPQPLKEQPALNDSRYALSSTLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC99):

(TCRCα;)
SEQ ID NO: 13
PYIQNPDPAVYQLRDSKSSDKFVCLFTDFDSQINVSQSKDSDVYITDKCV
LDMRSMDRKKNSAVAWSNKSDFTCANAFNNSIIPEDTFFPSPESSC,
and
(TCRCβ;)
SEQ ID NO: 14
EDLKNVFPPEVAVFEPSKAEISRTQEATLVCLATGFYPPHVELSWWVNGK
EVHDGVCTDPQPLKEQPALNDSRYALSSRLSVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC20):

(TCRCα;)
SEQ ID NO: 15
PYIQNPFPAVYQLRDSKSSDKFVCLFTDFDSQINVSQSKDSDVYITDKCV
LDMRSMDFKSNSAVAWSNKSDFTCANAFNNSIIPEDTWFPSPESSC,
and
(TCRCβ;)
SEQ ID NO: 16
EDLKNVFPPEVAVFEPSKAEISLTQKATLVCLATGFYPPHVELSWWVNGK
EVHDGVCTDPQPLKEQPALNDSRYALSSRLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC56):

(TCRCα;)
SEQ ID NO: 17
PYIQNPDPAVYQLRDSKSSDKFVCLFTDFDSQINVSQSKDSDVYITDKCV
LDMQDMDFKSNSAVAWSNKSDFTCANAFNNSIIPEDTFFPSPESSC,
and
(TCRCβ;)
SEQ ID NO: 18
EDLKNVFPPEVAVFEPSKAEISRTQKATLVCLATGFYPPHVELSWWVNGK
EVHKGVCTDPQPLKEQPALNDSRYALSSRLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC100):

(TCRCα;)
SEQ ID NO: 19
PYIQNPDPAVYQLRDSKSSDKFVCLFTHFDSQINVSQSKDSDVYITDKCV
LDMRSMDRKKNSAVAWSNKSDFTCANAFNNSIIPEDTFFPSPESSC,
and
(TCRCβ;)
SEQ ID NO: 20
EDLKNVFPPEVAVFEPSKAEISRTQEATLVCLATGFYPPHVELSWWVNGK
EVHDGVCTDPQPLKEQPALNDSRYALSSRLSVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desC128):

(TCRCα;)
SEQ ID NO: 21
PYIQNPRPAVYQLRDSKSSDKFVCLFTDFDSQINVSQSKDSDVYITDKCV
LDMRSMDFKSNSAVAWSNKSDFTCANAFNNSIIPEDTKFPSPESSC,
and
(TCRCβ;)
SEQ ID NO: 22
EDLKNVFPPEVAVFEPSKAEISDTQKATLVCLATGFYPPHVELSWWVNGK
EVHDGVCTDPQPLKEQPALNDSRYALSSRLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (combiC12).

(TCRCα;)
SEQ ID NO: 23
PYIQNPQPAVYQLRDSKSSDKFVCLFTDFDSQINVSQSKDSDVYITDKCV
LDMRSMDFKSNRAVAWSNKSDFTCANAFNNSIIPEDTKFPSPESSC,
and
(TCRCβ;)
SEQ ID NO: 24
EDLKNVFPPEVAVFEPSKAEISLTQKATLVCLATGFYPPHVELSWWVNGK
EVHDGVCTDPQPLKEQPALNDSRYALSSTLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (combiC26).

(TCRCα;)
SEQ ID NO: 25
PYIQNPQPAVYQLRDSKSSDKFVCLFTHFDSQINVSQSKDSDVYITDKCV
LDMRSMDRKKNRAVAWSNKSDFTCCANAFNNSIIPEDTKFPSPESSC,
and
(TCRCβ;)
SEQ ID NO: 26
EDLKNVFPPEVAVFEPSKAEISLTQEATLVCLATGFYPPHVELSWWVNGK
EVHDGVCTDPQPLKEQPALNDSRYALSSTLSVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (combiC48).

(TCRCα;)
SEQ ID NO: 27
PYIQNPRPAVYQLRDSKSSDKFVCLFTDFDSQINVSQSKDSDVYITDKCV
LDMRSMDFKSNRAVAWSNKSDFTCANAFNNSIIPEDTKFPSPESSC,
and
(TCRCβ;)
SEQ ID NO: 28
EDLKNVFPPEVAVFEPSKAEISDTQKATLVCLATGFYPPHVELSWWVNGK
EVHDGVCTDPQPLKEQPALNDSRYALSSSLRVSATFWQDPRNHFRCQVQF
YGLSENDEWTQDRAKPVTQIVSAEAWGRADC.

In some embodiments, the modified TCRCα domain and modified TCRCβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (combiC25):

(TCRCα;)
SEQ ID NO: 29
PYIQNPQPAVYQLRDSKSSDKFVCLFTDFDSQINVSQSKDSDVYITDKCV
LDMRSMDRKKNRAVAWSNKSDFTCANAFNNSIIPEDTKFPSPESSC,
and
(TCRCβ;)
SEQ ID NO: 30
EDLKNVFPPEVAVFEPSKAEISLTQEATLVCLATGFYPPHVELSWWVNGK
EVHDGVCTDPQPLKEQPALNDSRYALSSTLSVSATFWQDPRNHFRCQVQF
YGLSENDEWTQDRAKPVTQIVSAEAWGRADC.

In some embodiments, the one or more amino acid substitutions of each the bioorthogonal third and fourth interface are comprised in a conserved region of each of the modified TCRVα or fragment thereof and modified TCRVβ or fragment thereof.

In some embodiments, the conserved region of each of the modified TCRVα or fragment thereof and modified TCRVβ or fragment thereof comprises amino acid Kabat positions 1-24, 32-48, 62-92, and/or 105-116 in the TCRVα or fragment thereof, and/or amino acid Kabat positions 1-24, 32-48, 65-94, and/or 107-116 in the TCRVβ or fragment thereof, such as described in plueckthun.bioc.uzh.ch/antibody/Numbering/NumFrame.html and Thomas et al. 2019 Nature Communications 10:4451, the disclosures of each of which are incorporated herein by reference.

In some embodiments, the one or more substitutions of the bioorthogonal third interface and/or fourth interface comprise paired (e.g., bioorthogonal) knob-into-hole substitutions, charge swap substitutions, charge to hydrophobic substitutions, polar to cation-pi interaction, or any combination thereof.

In some embodiments, the one or more substitutions of the bioorthogonal third interface comprise substitution at amino acid position 31, 35, 37, 40, 41, 43, 45, 48, 86, 100, 101, 103, 105 and/or 108 (Kabat 109), wherein the numbering corresponds to PDB numbering of the reference TCRVα amino acid sequence of PDB: 2F53_D. Additional non-limiting examples of the one or more substitutions of the bioorthogonal third interface include any amino acid position and/or residue change as described in FIGS. 14A-14D and 15; and Tables 1-9.

In some embodiments, the one or more substitutions of the bioorthogonal third interface comprise substitution at amino acid positions 37 and/or 108 (Kabat 109), wherein the numbering corresponds to PDB numbering of the reference TCRVα amino acid sequence of PDB: 2F53_D (e.g., PDB numbering within residues-1 to 191 of the reference TCRVα amino acid sequence of 2F53_D (Chain D) in the Protein Data Bank (rcsb.org/sequence/2F53)).

Residues −1 to 191 of 2F53 D(TCRα chain,
Chain D):
SEQ ID NO: 31
MKQEVTQIPAALSVPEGENLVLNCSFTDSAIYNLQWFRQDPGKGLTSLLL
IPFWQREQTSGRLNASLDKSSGRSTLYIAASQPGDSATYLCAVRPTSGGS
YIPTFGRGTSLIVHPYIQNPDPAVYQLRDSKSSDKSVCLFTDFDSQTNVS
QSKDSDVYITDKCVLDMRSMDFKSNSAVAWSNKSDFACANAFN

In some embodiments, the one or more substitutions of the bioorthogonal third interface comprise 37Y, 37K, 37D, 37L, 37V, or 108K (Kabat 109K).

In some embodiments, the one or more substitutions of the bioorthogonal third interface comprise Q37Y, Q37K, Q37D, Q37L, Q37V, or S108K (Kabat S109K).

In some embodiments, the one or more substitutions of the bioorthogonal fourth interface comprise substitution at amino acid position 7, 8, 9, 29, 31, 33, 35 (Kabat 37), 38 (Kabat 40), 39, 41, 42, 43, 86, 99, 101, 102, 104, 107 and/or 150, wherein the numbering corresponds to PDB numbering of the reference TCRVβ amino acid sequence of PDB: 2F53_E. Additional non-limiting examples of the one or more substitutions of the bioorthogonal fourth interface include any amino acid position and/or residue change as described in FIGS. 14A-14D and 15; and Tables 1-9.

In some embodiments, the one or more substitutions of the bioorthogonal fourth interface comprise substitution at amino acid position 35 (Kabat 37) and/or 38 (Kabat 40) wherein the numbering corresponds to PDB numbering of the reference TCRVα amino acid sequence of PDB: 2F53_E (e.g., PDB numbering within residues-1 to 241 of the reference TCRVβ amino acid sequence of 2F53_E (Chain E) in the Protein Data Bank (rcsb.org/sequence/2F53)).

Residues −1 to 241 of 2F53 E(TCRβ chain,
Chain E):
SEQ ID NO: 32
NAGVTQTPKFQVLKTGQSMTLQCAQDMNHEYMSWYRQDPGMGLRLIHYSV
SVGMTDQGEVPNGYNVSRSTTEDFPLRLLSAAPSQTSVYFCASSYVGNTG
ELFFGEGSRLTVLEDLKNVFPPEVAVFEPSEAEISHTQKATLVCLATGFY
PDHVELSWWVNGKEVHSGVCTDPQPLKEQPALNDSRYALSSRLRVSATFW
QDPRNHFRCQVQFYGLSENDEWTQDRAKPVTQIVSAEAWGRAD

In some embodiments, one or more substitutions of the bioorthogonal fourth interface comprise 35K, 35Y, 35D, 35M (Kabat 37K, 37Y, 37D, 37M), or 38E (Kabat 40E).

In some embodiments, the one or more substitutions of the bioorthogonal fourth interface comprise Q35K, Q35Y, Q35D, Q35M, (Kabat Q37K, Q37Y, Q37D, Q37M), or G38E (Kabat G40E).

In some embodiments, the one or more substitutions of the bioorthogonal third interface and the bioorthogonal fourth interface comprise 37K in the third interface and 35Y (Kabat 37Y) in the fourth interface (e.g., desV30).

In some embodiments, the one or more substitutions of the bioorthogonal third interface and the bioorthogonal fourth interface comprise 37K in the third interface and 35Y (Kabat 37Y) in the fourth interface (e.g., desV11).

In some embodiments, the one or more substitutions of the bioorthogonal third interface and the bioorthogonal fourth interface comprise 37K in the third interface and 35D (Kabat 37D) in the fourth interface (e.g., desV31).

In some embodiments, the one or more substitutions of the bioorthogonal third interface and the bioorthogonal fourth interface comprise 37D in the third interface and 35K (Kabat 37K) in the fourth interface (e.g., desV32).

In some embodiments, the one or more substitutions of the bioorthogonal third interface and the bioorthogonal fourth interface comprise 37V in the third interface and 35M (Kabat 37M) in the fourth interface (e.g., desV40).

In some embodiments, the one or more substitutions of the bioorthogonal third interface and the bioorthogonal fourth interface comprise 108K (Kabat 109K) in the third interface and 38E (Kabat 40E) in the fourth interface (e.g., desV58).

In some embodiments, the one or more substitutions of the bioorthogonal third interface and the bioorthogonal fourth interface comprise 37L and 35M (Kabat 37M) in the third interface and 139E in the fourth interface (e.g., desV38).

In some embodiments, the modified TCRVα domain and modified TCRVβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV30):

(TCRVα; SEQ ID NO: 33)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 34)
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL.

In some embodiments, the modified TCRVα domain and modified TCRVβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV11):

(TCRVα; SEQ ID NO: 35)
QEVTQIPAALSVPEGENLVLNCSFTDSAIYNLQWFRYDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 36)
GVTQTPKFQVLKTGQSMTLQCAQDMNHEYMSWYRKDPGMGLRLIHYSVA
IQTTDQGEVPNGYNVSRSTIEDFPLRLLSAAPSQTSVYFCASSYVGDTG
ELFFGEGSRLTVL.

In some embodiments, the modified TCRVα domain and modified TCRVβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV31):

(TCRVα; SEQ ID NO: 37)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 38)
GVTQTPKFQVLKTGQSMTLQCAQDMNHEYMSWYRDDPGMGLRLIHYSVA
IQTTDQGEVPNGYNVSRSTIEDFPLRLLSAAPSQTSVYFCASSYVGDTG
ELFFGEGSRLTVL;

In some embodiments, the modified TCRVα domain and modified TCR Vβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV32):

(TCRVα; SEQ ID NO: 39)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 40)
GVTQTPKFQVLKTGQSMTLQCAQDMNHEYMSWYRKDPGMGLRLIHYSVA
IQTTDQGEVPNGYNVSRSTIEDFPLRLLSAAPSQTSVYFCASSYVGDTG
ELFFGEGSRLTVL.

In some embodiments, the modified TCRVα domain and modified TCRVβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV40):

(TCRVα; SEQ ID NO: 41)
QEVTQIPAALSVPEGENLVLNCSFTDSAIYNLQWFRVDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 42)
GVTQTPKFQVLKTGQSMTLQCAQDMNHEYMSWYRMDPGMGLRLIHYSVA
IQTTDQGEVPNGYNVSRSTIEDFPLRLLSAAPSQTSVYFCASSYVGDTG
ELFFGEGSRLTVL;

In some embodiments, the modified TCRVα domain and modified TCRVβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV58):

(TCRVα; SEQ ID NO: 43)
QEVTQIPAALSVPEGENLVLNCSFTDSAIYNLQWFRQDPGKGLTSLLLI
SPWQREQTSGRLNASLDKSSGRSTLYIAASQPGDSATYLCAVRPLLDGT
YIPTFGRGTKLIVH,
and
(TCRVβ; SEQ ID NO: 44)
GVTQTPKFQVLKTGQSMTLQCAQDMNHEYMSWYRQDPEMGLRLIHYSVA
IQTTDQGEVPNGYNVSRSTIEDFPLRLLSAAPSQTSVYFCASSYVGDTG
ELFFGEGSRLTVL.

In some embodiments, the modified TCRVα domain and modified TCRVβ domain or fragment thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV38):

(TCRVα; SEQ ID NO: 45)
QEVTQIPAALSVPEGENLVLNCSFTDSAIYNLQWFRLDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 46)
GVTQTPKFQVLKTGQSMTLQCAQDMNHEYMSWYRMDPGMGLRLIHYSVA
IQTTDQGEVPNGYNVSRSTIEDFPLRLLSAAPSQTSVYFCASSYVGDTG
ELFFGEGSRLTVL.

The synthetic TCR molecules of the present invention may comprise any modified TCRCα, TCRCβ, TCRVα, and/or TCRVβ described herein, in any combination, with or without one or more (e.g., two, three, four, five, six, seven, eight, nine, ten, etc.) additional amino acid modifications (e.g., deletions, insertions, and/or substitutions), such as but not limited to any additional substitutions described herein, e.g., such as in FIGS. 14A-14D and FIG. 15, and Tables 1-9.

In some embodiments, the one or more substitutions of each bioorthogonal interface comprise 124D (Kabat 122D), 179R, and 205K in the first interface, 139L and 195T in the second interface, 37K in the third interface, and 35Y (Kabat 37Y) in the fourth interface (e.g., desV30combiC12).

In some embodiments, the one or more substitutions of each bioorthogonal interface comprise 124Q (Kabat 122Q), 175R, 177K, 179S and 205K in the first interface, 139L, 142E, 195T and 197S in the second interface, 37K in the third interface, and 35Y (Kabat 37Y) in the fourth interface (e.g., desV30combiC25).

In some embodiments, the one or more substitutions of each bioorthogonal interface comprise 124Q (Kabat 122Q), 145H, 175R, 177K, 179R and 205K in the first interface, 139L, 142E, 195T and 197S in the second interface, 37K in the third interface, and 35Y (Kabat 37Y) in the fourth interface (e.g., desV30combiC26).

In some embodiments, the one or more substitutions of each bioorthogonal interface comprise 124R (Kabat 122R), 179R and 205K in the first interface, 139D and 195S in the second interface, 37K in the third interface, and 35Y (Kabat 37Y) in the fourth interface (e.g., des V30combiC48).

In some embodiments, the one or more substitutions of each bioorthogonal interface comprise 124Q (Kabat 122Q), 179S and 205K in the first interface, 139E and 195S in the second interface, 37K in the third interface, and 35Y (Kabat 37Y) in the fourth interface (e.g., desV30combiC46).

In some embodiments, the modified TCRCα domain, modified TCRCβ domain, TCRVα domain, modified TCRVβ domain, or fragments thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV30combiC12):

(TCRCα; SEQ ID NO: 23)
PYIQNPQPAVYQLRDSKSSDKFVCLFTDFDSQINVSQSKDSDVYITDKC
VLDMRSMDFKSNRAVAWSNKSDFTCANAFNNSIIPEDTKFPSPESSC,
(TCRCβ; SEQ ID NO: 24)
EDLKNVFPPEVAVFEPSKAEISLTQKATLVCLATGFYPPHVELSWWVNG
KEVHDGVCTDPQPLKEQPALNDSRYALSSTLRVSATFWQDPRNHFRCQV
QFYGLSENDEWTQDRAKPVTQIVSAEAWGRADC
(TCRVα; SEQ ID NO: 33)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 34)
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL.

In some embodiments, the modified TCRCα domain, modified TCRCβ domain, TCRVα domain, modified TCRVβ domain, or fragments thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV30combiC25):

(TCRCα; SEQ ID NO: 29)
PYIQNPQPAVYQLRDSKSSDKFVCLFTDFDSQINVSQSKDSDVYITDKC
VLDMRSMDRKKNRAVAWSNKSDFTCANAFNNSIIPEDTKFPSPESSC,
(TCRCβ; SEQ ID NO: 30)
EDLKNVFPPEVAVFEPSKAEISLTQEATLVCLATGFYPPHVELSWWVNG
KEVHDGVCTDPQPLKEQPALNDSRYALSSTLSVSATFWQDPRNHFRCQV
QFYGLSENDEWTQDRAKPVTQIVSAEAWGRADC,
(TCRVα; SEQ ID NO: 33)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 34)
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL.

In some embodiments, the modified TCRCα domain, modified TCRCβ domain, TCRVα domain, modified TCRVβ domain, or fragments thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV30combiC26):

(TCRCα; SEQ ID NO: 25)
PYIQNPQPAVYQLRDSKSSDKFVCLFTHEDSQINVSQSKDSDVYITDKC
VLDMRSMDRKKNRAVAWSNKSDFTCCANAFNNSIIPEDTKFPSPESSC,
(TCRCβ; SEQ ID NO: 26)
EDLKNVFPPEVAVFEPSKAEISLTQEATLVCLATGFYPPHVELSWWVNG
KEVHDGVCTDPQPLKEQPALNDSRYALSSTLSVSATFWQDPRNHFRCQV
QFYGLSENDEWTQDRAKPVTQIVSAEAWGRADC,
(TCRVα; SEQ ID NO: 33)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 34)
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL.

In some embodiments, the modified TCRCα domain, modified TCRCβ domain, TCRVα domain, modified TCRVβ domain, or fragments thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV30combiC48):

(TCRCα; SEQ ID NO: 27)
PYIQNPRPAVYQLRDSKSSDKFVCLFTDFDSQINVSQSKDSDVYITDKC
VLDMRSMDFKSNRAVAWSNKSDFTCANAFNNSIIPEDTKFPSPESSC,
(TCRCβ; SEQ ID NO: 28)
EDLKNVFPPEVAVFEPSKAEISDTQKATLVCLATGFYPPHVELSWWVNG
KEVHDGVCTDPQPLKEQPALNDSRYALSSSLRVSATFWQDPRNHFRCQV
QFYGLSENDEWTQDRAKPVTQIVSAEAWGRADC,
(TCRVα; SEQ ID NO: 33)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 34)
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL.

In some embodiments, the modified TCRCα domain, modified TCRCβ domain, TCRVα domain, modified TCRVβ domain, or fragments thereof comprises, consists essentially of, or consists of an amino acid sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of (desV30combiC46):

(TCRCα; SEQ ID NO: 7)
PYIQNPRPAVYQLRDSKSSDKFVCLFTDFDSQINVSQSKDSDVYITDKC
VLDMRSMDFKSNRAVAWSNKSDFTCANAFNNSIIPEDTKFPSPESSC,
(TCRCβ; SEQ ID NO: 8)
EDLKNVFPPEVAVFEPSKAEISETQKATLVCLATGFYPPHVELSWWVNG
KEVHDGVCTDPQPLKEQPALNDSRYALSSSLRVSATFWQDPRNHFRCQV
QFYGLSENDEWTQDRAKPVTQIVSAEAWGRADC,
(TCRVα; SEQ ID NO: 33)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 34)
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL.

The synthetic TCR molecules of the present invention may be in soluble form (e.g., wherein the synthetic TCR molecule is devoid of a transmembrane domain) or in cellular form (e.g., as a chimeric antigen receptor (CAR) expressed on the surface of a cell).

In some embodiments, a synthetic TCR molecule of the present invention may further comprise a second TCRCα, TCRCβ, TCRVα, and TCRVβ or fragments thereof, each comprising an N-terminus and a C-terminus and a first, second, third and fourth interface, respectively, wherein at least a portion of each of the second TCRVα and TCRVβ together form a variable portion with binding specificity to a second target antigen that is different from the first target antigen.

In some embodiments, the second TCRCα, TCRCβ, TCRVa, and TCRVβ or fragments thereof are unmodified (e.g., wildtype).

In some embodiments, the second TCRCα, TCRCβ, TCRVa, and TCRVβ or fragments thereof are modified (e.g., modified to comprise bioorthogonal interfaces, e.g., wherein the second TCRCα and TCRCβ first and second interfaces selectively bind to each other and/or the second TCRVα and TCRVβ third and fourth interfaces selectively bind to each other, e.g., selectively bind to each other as compared to unmodified interfaces).

In some embodiments, the C-terminus or N-terminus of the modified TCRCα, TCRCβ, TCRVα, and/or TCRVβ or fragment thereof with an interface which is bioorthogonal to a corresponding interface, is linked to the N-terminus or C-terminus of the second (e.g., modified or unmodified) TCRCα, TCRCβ, TCRVα, and/or TCRVβ or fragment thereof.

In some embodiments, the synthetic TCR molecule of the present invention is a bispecific TCR.

In some embodiments, the synthetic TCR molecule of the present invention may further comprise an antibody Fc or fragment thereof comprising an N-terminus and a C-terminus, wherein the C-terminus of the modified TCRCα or fragment thereof with a first interface which is bioorthogonal to the second interface, or the C-terminus of the TCRCβ or fragment thereof with a second interface which is bioorthogonal to the first interface, is linked to an N-terminus of the antibody Fc or fragment thereof, and optionally wherein the C-terminus of the second (modified or unmodified) TCRCα or fragment thereof or the C-terminus of the second TCRCβ or fragment thereof is linked to another N-terminus of the antibody Fc or fragment thereof.

An antibody Fc or fragment thereof of the present invention may be any known or as yet discovered and/or generated natural (e.g., wildtype), synthetic, and/or modified Fc. In some embodiments, the antibody Fc or fragment thereof of the present invention comprises an IgE, IgA, IgM, IgD, or IgG Fc or fragment thereof. In some embodiments, the antibody Fc or fragment thereof of the present invention comprises an IgG Fc or fragment thereof.

In some embodiments, the antibody Fc or fragment thereof comprises two or more bioorthogonal domains, each comprising one or more modifications (e.g., amino acid substitutions) and which selectively bind to each other via the one or more modifications (e.g., wherein the Fc domains are bioorthogonal, e.g., selectively bind to each other as compared to unmodified interfaces). In some embodiments, the antibody Fc or fragment thereof of the present invention may comprise any antibody Fc or fragment thereof as described in Leaver-Fay et al. 2016 Structure 24 (4): 641-651 and/or US Patent Application No. US 2021/0054103, the disclosures of each of which are incorporated herein.

In some embodiments, the synthetic TCR molecule of the present invention may further comprise an antibody or antibody fragment (e.g., an antibody Fab, a Fab′, a F(ab′)2, a Fv, a disulfide linked Fv, a single chain antibody (scFv), a single domain antibody (dAb), a diabody, a nanobody, and/or an affibody or fragment thereof) e.g., in particular embodiments, an antibody scFv) comprising an N-terminus and a C-terminus, wherein the C-terminus of the modified TCRVβ or fragment thereof with a fourth interface which is bioorthogonal to the third interface, is linked to an N-terminus of the antibody or antibody fragment, and optionally wherein the C-terminus of the second (modified or unmodified) TCRCβ or fragment thereof is linked to another N-terminus of the antibody or antibody fragment.

In some embodiments, the synthetic TCR molecule of the present invention may further comprise a T cell inhibitory domain (e.g., PD-1, ITIM) or fragment thereof.

In some embodiments, the synthetic TCR molecule of the present invention may further comprise a T cell signaling domain (e.g., CD32) or fragment thereof and/or a T cell co-stimulatory domain (e.g., CD28, 4-1BB).

In some embodiments, the synthetic TCR molecule of the present invention may be devoid of a signaling domain or fragment thereof.

In some embodiments, the synthetic TCR molecule of the present invention may be devoid of a costimulatory domain or fragment thereof.

In some embodiments, the synthetic TCR molecule of the present invention may comprise a T cell signaling domain or fragment thereof and a T cell co-stimulatory domain.

In some embodiments, wherein the synthetic TCR molecule of the present invention may further comprise a second TCRCα, TCRCβ, TCRVα, and TCRVβ or fragments thereof, each comprising an N-terminus and a C-terminus, wherein at least a portion of each of the second TCRVα and TCRVβ together form a variable portion with binding specificity to a second target antigen that is different from the first target antigen, the C-terminus or N-terminus of the modified TCRCα, TCRCβ, TCRVα, and/or TCRVβ or fragment thereof with an interface which is bioorthogonal to a corresponding interface, is linked to the N-terminus or C-terminus of the second (e.g., modified or unmodified) TCRCα, TCRCβ, TCRVα, and/or TCRVβ or fragment thereof.

In some embodiments, the synthetic TCR molecule of the present invention may further comprise a hinge region (e.g., CD8 hinge, CD4 hinge), a transmembrane domain, a linker, a costimulatory domain (e.g., CD28, 4-1BB, etc.) and/or an scFv and/or Fab (e.g., wherein the synthetic TCR molecule is a chimeric antigen receptor (CAR).

In some embodiments, the synthetic TCR molecule of the present invention binds a major histocompatibility complex (MHC) (e.g., in vivo and/or in vitro).

In some embodiments, the bioorthogonal first and second interface of the present invention, when selectively bound to each other, and/or the bioorthogonal third and fourth interface, when selectively bound to each other, have a melting temperature of about 55° C. to about 85° C. Tm (e.g., about 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85° C. or any value or range therein, e.g., about 70 to about 80, e.g., for TCRC designs, e.g., about 60 to about 65, e.g., for TCRV designs, about 62 to about 68, e.g., for TCRVC designs).

In some embodiments, the synthetic TCR molecule of the present invention may further comprise a detectable moiety.

In some embodiments, the synthetic TCR molecule of the present invention may further comprise an effector molecule selected from the group consisting of a drug, a toxin, a small molecule, a radioactive molecule, a photoactivatable molecule, an antibody, a cytokine, an oncolytic virus, an enzyme, a nanoparticle, a biomaterial, a scaffold and any combination thereof.

A target antigen of the present invention (e.g., the first and/or the second target antigen) may be any target antigen. Without wishing to be bound to theory, the invention as described encompasses modifications to the TCR constant domain and/or conserved regions of TCR variable (variant) domains and accordingly do not modify the TCR variable portion with binding specificity to the target antigen. Accordingly, the TCR molecules of the present invention are not limited to any particular class of target antigens. Non-limiting examples of target antigens include a cancer antigen, a virus antigen, and/or a bacterial antigen, or any combination thereof. For example, in some embodiments, the target antigen may be (cancer antigens) NY-ESO-1, MAGE_A3, MAGE_A4, gp100, MART-1/MelanA, KRas, p53, WT1, hTERT etc., (viral antigens) EBV, HTLV-1, HPV E6, HPV E7, HIV

Also provided is a cell (e.g., an isolated cell) comprising a synthetic TCR molecule, vector, nucleic acid molecule, and/or composition of this invention, singly or in any combination. The isolated cell may be from any source (e.g., mammalian, insect, synthetic, cell-like particle, etc.). In some embodiments, the cell may be selected from the group consisting of an αβT cell (e.g., a CD4+αβT cell, a CD8+αβT cell), a natural killer (NK) cell, a cytotoxic T lymphocyte (CTL), a regulatory T cell, a natural killer T (NKT) cell, a Th17 cell, a γδT cell, a neutrophil, a macrophage, an artificial cell (e.g., cell-like particle) and any combination thereof. In some embodiments, the isolated cell may be an in vitro cell (e.g., an immortalized cell, e.g., a cell line). In some embodiments, the isolated cell may be an ex vivo cell from a subject (e.g., a human patient).

In some embodiments, the isolated cell may comprise a synthetic TCR molecule of the present invention, wherein the synthetic TCR molecule is expressed on the surface of the cell.

In some embodiments, the isolated cell for of the present invention may comprise a chimeric antigen receptor (CAR) that is different from the synthetic TCR molecule (e.g., that has specificity for a target antigen that is different from the first and/or second target antigen of the synthetic TCR molecule).

The present invention further provides an isolated nucleic acid molecule encoding a synthetic TCR molecule of the invention. In some embodiments, a nucleic acid molecule of this invention may be a cDNA molecule. In some embodiments, a nucleic acid molecule of this invention may be an mRNA molecule.

Also provided is a vector, plasmid or other nucleic acid construct (e.g., a virus vector, e.g., a virus-like particle) comprising the isolated nucleic acid molecule of this invention.

A vector can be any suitable means for delivering a polynucleotide to a cell. A vector of this invention can be an expression vector that contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid molecule or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a synthetic TCR molecule of this invention is produced in the cell (e.g., a host cell). In addition, the vector of this invention can be in a cell, which can be a cell expressing the nucleic acid of the vector whereby a synthetic TCR molecule of this invention is produced in the cell. It is also contemplated that the nucleic acid molecules and/or vectors of this invention can be present in a host organism (e.g., a transgenic organism), which expresses the nucleic acids of this invention and produces a synthetic TCR molecule of this invention. In some embodiments, the vector is a plasmid, a viral vector, a bacterial vector, an expression cassette, a transformed cell, or a nanoparticle. For example, in some embodiments a synthetic TCR molecule of the present invention may be used in combination (e.g., in scaffold(s) and/or conjugated with) other molecules such as, but not limited to, nanoparticles, e.g., as delivery devices.

Types of nanoparticles of this invention for use as a vector and/or delivery device include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art.

Also provided are compositions comprising a synthetic TCR molecule, nucleic acid molecule, vector, and/or isolated cell of the present invention. In some embodiments, a composition of the present invention may further comprise a pharmaceutically acceptable carrier, diluent and/or adjuvant (e.g., a pharmaceutical composition, e.g., a pharmaceutical formulation).

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the present invention provides a pharmaceutical composition comprising a synthetic TCR molecule, nucleic acid molecule (e.g., an mRNA molecule), vector, cell, and/or composition of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions comprising a synthetic TCR molecule, nucleic acid molecule (e.g., an mRNA molecule), vector, cell, and/or composition of the present invention may be formulated by any means known in the art. Such compositions, especially vaccines and/or therapeutics, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise a synthetic TCR molecule, nucleic acid molecule (e.g., an mRNA molecule), vector, cell, and/or composition of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition comprising a synthetic TCR molecule, nucleic acid molecule, vector, cell, and/or composition of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc.

In some embodiments, a pharmaceutical composition comprising a synthetic TCR molecule, nucleic acid molecule (e.g., an mRNA molecule), vector, cell, and/or composition of the present invention, and a pharmaceutically acceptable carrier may further comprise an adjuvant. As used herein, “suitable adjuvant” describes an adjuvant capable of being combined with a synthetic TCR molecule, nucleic acid molecule (e.g., an mRNA molecule), vector, cell, and/or composition of the present invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24 (6): 766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

### Methods

The synthetic TCR molecule, nucleic acid molecule, vector, cell, and/or composition of the present invention is intended for use as therapeutic agents and immunological reagents, for example, as antigens, immunogens, prophylactics, therapeutics, vaccines, and/or delivery vehicles. Accordingly, the present invention can be practiced for prophylactic, therapeutic and/or diagnostic purposes. The compositions described herein can be formulated for use as reagents and/or for administration in a pharmaceutical carrier in accordance with known techniques. See, e.g., Remington, The Science and Practice of Pharmacy (latest edition).

Accordingly, another aspect of the invention provides a method of expressing a synthetic TCR molecule in a cell, comprising contacting the cell with the nucleic acid molecule, vector, and/or composition of the present invention. In some embodiments, the cell is in a subject (e.g., a human patient).

Another aspect of the invention provides a method of treating a disorder in a subject, comprising administering to the subject an effective amount of a synthetic TCR molecule, nucleic acid molecule, vector, isolated cell, and/or composition of the present invention, wherein the synthetic TCR binds an antigen associated with the disorder (e.g., a cancer antigen, a viral antigen, a bacterial antigen, or any combination thereof).

The disorder may be any disorder that expresses and/or is associated with an antigen, wherein the antigen is the target antigen (e.g., first and/or second target antigen) of a synthetic TCR molecule of the present invention. Non-limiting examples of disorders contemplated in the invention include cancer (e.g., melanoma, lymphoma, leukemia, pancreatic cancer), viral infection, bacterial infection, autoimmune disease, cellular senescence, or any combination thereof.

Another aspect of the present invention provides a method of producing a synthetic T-cell receptor (TCR) molecule, comprising: (a) providing a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen; (b) introducing one or more amino acid substitutions into the first and second interface and/or into the third and fourth interface, wherein the one or more amino acid substitutions modify the first and the second interface and/or the third and fourth interface such that the interfaces are bioorthogonal to each other (e.g., such that the bioorthogonal interfaces selectively bind to each other, e.g., selectively bind to each other as compared to unmodified interfaces); thereby producing a synthetic TCR molecule (e.g., a synthetic TCR molecule of the present invention).

Another aspect of the present invention provides a method of enhancing stability (e.g., in vitro and/or in vivo) of a T-cell receptor (TCR) molecule, comprising: (a) providing a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen; (b) introducing one or more amino acid substitutions into the first and second interface and/or into the third and fourth interface, wherein the one or more amino acid substitutions modify the first and the second interface and/or the third and fourth interface such that the interfaces are bioorthogonal to each other (e.g., such that the bioorthogonal interfaces selectively bind to each other, e.g., selectively bind to each other as compared to unmodified interfaces), thereby enhancing the stability of the synthetic TCR molecule; thereby producing a synthetic TCR molecule (e.g., a synthetic TCR molecule of the present invention), wherein the synthetic TCR molecule has enhanced stability (e.g., as compared to an unmodified TCR).

The synthetic TCR molecule of the present invention may be administered in any frequency, amount, and/or route as needed to elicit an effective prophylactic and/or therapeutic effect in a subject (e.g., in a subject in need thereof) as described herein. In certain embodiments, synthetic TCR molecule, nucleic acid molecule, vector, cell, and/or composition of the present invention is administered/delivered to the subject, e.g., systemically (e.g., intravenously). In particular embodiments, more than one administration (e.g., two, three, four or more administrations) may be employed to achieve the desired level of protein expression over a period of various intervals, e.g., daily, weekly, monthly, yearly, etc. The most suitable route in any given case will depend on the nature and severity of the condition being treated and on the nature of the particular delivery method that is being used. In embodiments wherein a vector is used, the vector will typically be administered in a liquid formulation by direct injection (e.g., stereotactic injection) to the desired region or tissues. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. For example, controlled release of parvovirus and AAV vectors is described in international patent publication WO 01/91803, which is incorporated by reference herein for these teachings.

Administration may be by any suitable means, such as intraperitoneally, intramuscularly, intranasally, intravenously, intradermally (e.g., by a gene gun), intrarectally and/or subcutaneously. The compositions herein may be administered via a skin scarification method, and/or transdermally via a patch or liquid. The compositions can be delivered subdermally in the form of a biodegradable material that releases the compositions over a period of time. As further non-limiting examples, the route of administration can be by inhalation (e.g., oral and/or nasal inhalation), oral, buccal (e.g., sublingual), rectal, vaginal, topical (including administration to the airways), intraocular, by parenteral (e.g., intramuscular [e.g., administration to skeletal muscle], intravenous, intra-arterial, intraperitoneal and the like), subcutaneous (including administration into the footpad), intrapleural, intracerebral, intrathecal, intraventricular, intra-aural, intra-ocular (e.g., intra-vitreous, sub-retinal, anterior chamber) and peri-ocular (e.g., sub-Tenon's region) routes or any combination thereof.

In some embodiments, the synthetic TCR molecule can be administered to a subject as a nucleic acid molecule, which can be a naked nucleic acid molecule or a nucleic acid molecule present in a vector (e.g., a delivery vector, which in some embodiments can be a cell (e.g., a CAR-expressing cell, e.g., a CAR-T cell). The nucleic acids and vectors of this invention can be administered orally, intranasally, parenterally (e.g., intravenously), by intramuscular injection, by intraperitoneal injection, transdermally, extracorporeally, topically or the like. In the methods described herein which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for expression of the polypeptides and/or fragments of this invention. The vector can be a commercially available preparation or can be constructed in the laboratory according to methods well known in the art.

Delivery of the nucleic acid or vector to cells can be via a variety of mechanisms, including but not limited to recombinant vectors including bacterial, viral, and fungal vectors, liposomal delivery agents, nanoparticles, and gene gun related mechanisms.

In some embodiments, the nucleic acid molecules encoding the synthetic TCR molecule of this invention can be part of a recombinant nucleic acid construct comprising any combination of restriction sites and/or functional elements as are well known in the art that facilitate molecular cloning and other recombinant nucleic acid manipulations. Thus, the present invention further provides a recombinant nucleic acid construct comprising a nucleic acid molecule encoding a synthetic TCR molecule of this invention. The nucleic acid molecule encoding the synthetic TCR molecule of this invention can be any nucleic acid molecule that functionally encodes the synthetic TCR molecule of this invention. To functionally encode the synthetic TCR molecule of this invention (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Non-limiting examples of expression control sequences that can be present in a nucleic acid molecule of this invention include promoters derived from metallothionine genes, actin genes, immunoglobulin genes, CMV, SV40, adenovirus, bovine papilloma virus, etc. A nucleic acid molecule encoding a selected synthetic TCR molecule of this invention can readily be determined based upon the genetic code for the amino acid sequence of the selected polypeptide and/or fragment of interest included in the synthetic TCR molecule of this invention, and many nucleic acids will encode any selected polypeptide and/or fragment. Modifications in the nucleic acid sequence encoding the polypeptide and/or fragment are also contemplated. Modifications that can be useful are modifications to the sequences controlling expression of the polypeptide and/or fragment to make production of the polypeptide and/or fragment inducible or repressible as controlled by the appropriate inducer or repressor. Such methods are standard in the art. The nucleic acid molecule and/or vector of this invention can be generated by means standard in the art, such as by recombinant nucleic acid techniques and/or by synthetic nucleic acid synthesis or in vitro enzymatic synthesis.

The nucleic acids and/or vectors of this invention can be transferred into a host cell (e.g., a prokaryotic or eukaryotic cell) by well-known methods, which vary depending on the type of cell host. For example, calcium chloride transfection is commonly used for prokaryotic cells, whereas calcium phosphate treatment, transduction, cationic lipid treatment and/or electroporation can be used for other cell hosts.

As another example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega, Madison, WI), as well as other liposomes developed according to procedures standard in the art. In addition, the nucleic acid or vector of this invention can be delivered in vivo by electroporation, the technology for which is available from Genetronics, Inc. (San Diego, CA) as well as by means of a SONOPORATION machine (ImaRx Pharmaceutical Corp., Tucson, AZ).

As another example, vector delivery can be via a viral system, such as a retroviral vector system, which can package a recombinant retroviral genome. The recombinant retrovirus can then be used to infect and thereby deliver to the infected cells nucleic acid encoding the polypeptide and/or fragment of this invention. The exact method of introducing the exogenous nucleic acid into mammalian cells is, of course, not limited to the use of retroviral vectors. Other techniques are widely available for this procedure including the use of adenoviral vectors, alphaviral vectors (e.g., VRPs), adeno-associated viral (AAV) vectors, lentiviral vectors, pseudotyped retroviral vectors and vaccinia viral vectors, as well as any other viral vectors now known or developed in the future. Physical transduction techniques can also be used, such as liposome delivery and receptor-mediated and other endocytosis mechanisms. This invention can be used in conjunction with any of these or other commonly used gene transfer methods.

The present invention further provides a kit comprising one or more compositions of this invention. It would be well understood by one of ordinary skill in the art that the kit of this invention can comprise one or more containers and/or receptacles to hold the reagents (e.g., synthetic TCR molecule, vectors, compositions, nucleic acids) of the kit, along with appropriate buffers and/or diluents and/or other solutions and directions for using the kit, as would be well known in the art. Such kits can further comprise adjuvants and/or other immunostimulatory or immunomodulating agents, as are well known in the art.

The compositions and kits of the present invention can also include other medicinal agents, pharmaceutical agents, carriers, diluents, immunostimulatory cytokines, etc. Actual methods of preparing such dosage forms are known, or will be apparent, to those skilled in this art.

Immunomodulatory compounds, such as immunomodulatory chemokines and cytokines (preferably, CTL inductive cytokines) can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, a viral adjuvant expresses the cytokine.

If ex vivo methods are employed, cells or tissues can be removed and maintained outside the body according to standard protocols well known in the art. The nucleic acids and vectors of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

An adjuvant for use with the present invention, such as any adjuvant disclosed herein, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science And Practice of Pharmacy (9^{th} Ed. 1995). In the manufacture of a pharmaceutical composition according to the invention, the compositions are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL [R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

Further, the composition can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1: Development of Bioorthogonal TCR Designs

This project aimed to develop bispecific TCR (bsTCR) therapeutics composed of two different TCRs to improve affinity and specificity toward cancer cells. To solve subunit mispairing, orthogonal interfaces between TCR α and β chains were designed so that each α chain paired with the correct β chain. The designed orthogonal TCRs can be employed to develop logic gated TCR drugs that enable precise recognition of specific cancer cells.

Studies on bispecific antibodies showed that bivalent recognition with two recognition domains greatly enhance binding avidity, and similarly that bispecific antibodies achieve increased selectivity by simultaneously engaging two different targets. By tuning the combination of TCR pairs and their total binding avidities, bsTCR therapeutics can achieve AND-gate Boolean logic operations, to provide target-cell killing only when the target cell expresses two specific antigens. The following aims were pursued:

### Computational Design of an Orthogonal Interface for Correct TCR Subunit Assembly:

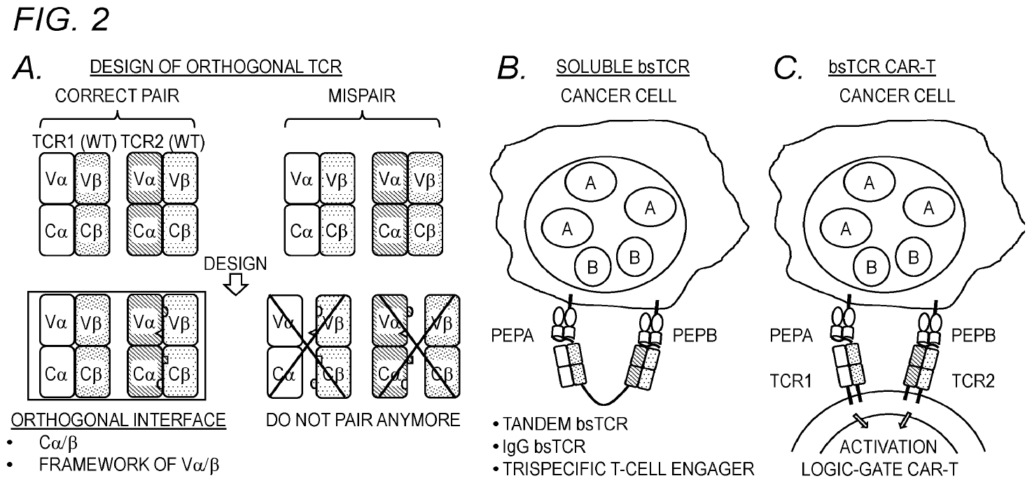

Designs for developing orthogonal TCR (as schematized in FIG. 2 panel A) were pursued by making mutations in the TCRα and β chains so that the resultant mutants interact with each other but no longer interact with their wild-type partners. To identify such mutations, a multistate design (MSD) protocol in Rosetta (refs 22, 31, 32) and a newly developed modeling pipeline, based on a second site suppressor (SSS) strategy (refs 33-35). Rosetta relies on rotamer-based sampling of amino acid side chains and an energy function accounting for atomic interactions, packing, and implicit solvation. Multistate design protocols in Rosetta have been previously used to create orthogonal interfaces between the heavy and light chains of bispecific antibodies. To ensure that the resultant designs are applicable to a broad set of TCRs, mutations were restricted to the TCR constant domains and the framework region of the variable domains. The designed TCRs were tested by being expressed in mammalian cells and validated with biochemical and biophysical molecular interaction analysis and X-ray crystallography. These designed orthogonal interfaces enable correct assembly of TCR subunits, which is important for the development of soluble and cellular bsTCR therapeutics, such as schematized in FIG. 2 panels B and C. Selected designs were validated with biochemical and biophysical analyses, including SDS-PAGE, nanoDSF, SPR, and X-ray crystallography.

Designs were initiated from a TCR-C (PDB: 6U07) designed by the Kuhlman lab with mutations that increase the melting temperature (Tm) of Cα/Cβ by 20° C.; the mutations improved assembly and stability of TCRs without affecting binding affinity and T-cell activation function (ref 26; incorporated herein by reference)²⁶. The expression levels and stability of the starting TCR-C (stC) as well as human (huC) and mouse TCR-C (moC), the most widely used pair to avoid TCR mispairing^{38,39} , was examined. Results indicated that the huC and moC were unstable and formed a smear-like pattern in SDS-PAGE due to glycosylation, unlike stTCR (FIG. 3 panel B). Moreover, the huCα/moCβ mispair had equivalent or higher Tm and increased expression level, compared with correct huC and moC pair, which indicated that more than half of the expressed proteins caused mispairing (FIG. 3 panel C). The design of a corresponding TCR-V was initiated from the 1G4 TCR (PDB: 2F53) which targets peptides from NY-ESO-1, a common antigen in melanomas (ref 40).

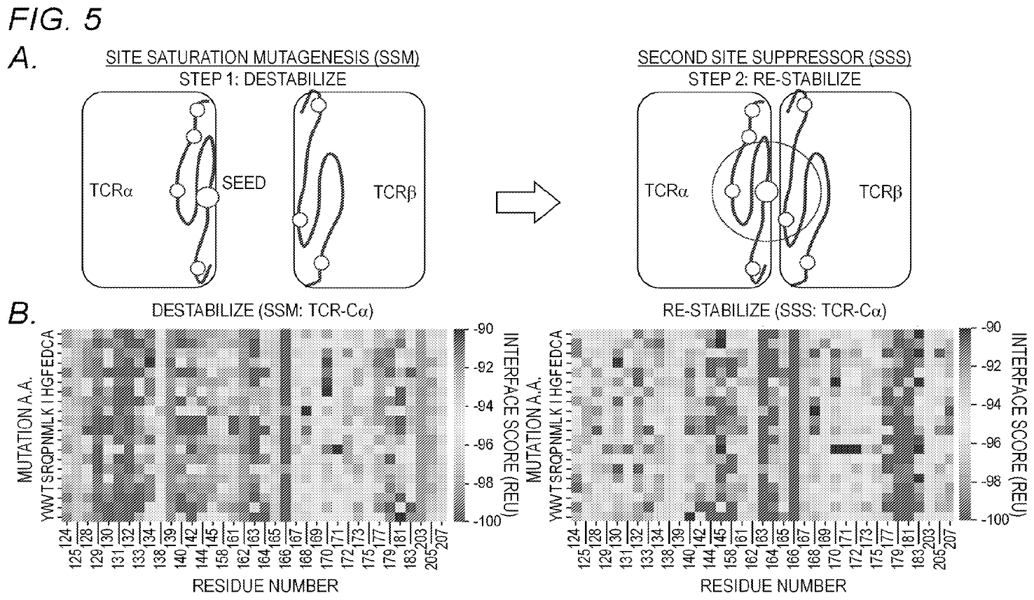

The MSD protocol creates explicit models of the MT/MT pair (i.e., mutant α chain with mutant β chain), MT/WT and WT/MT pair, and then searches for sequences that increase the energy gap between MT/MT and the two other undesired pairs (FIG. 4 panel A). The stability of the interface was evaluated based on the interface score term of Rosetta, which represents the change in Rosetta energy unit (REU) when the interface-forming chains are separated versus when they interact (FIG. 4 panel B). Focus was placed on residue clusters in different regions of the interface (48 clusters for TCR-C, 12 clusters for TCR-V). 140 resultant designs (420 states) with favorable energies and structure models were selected for experimental characterization.

Although the use of MSD algorithms provided some designs, designs were restricted to a small number of residues (˜12) for each simulation, which prevented an unbiased and comprehensive survey of the entire TCR interface. Therefore, a novel Rosetta modeling pipeline was developed by combining a computational approach with a prior design strategy, the Second Site Suppressor (SSS) strategy (refs 33-35). Site saturation mutagenesis (SSM) was computationally performed to the residues in the TCR α and β interface and seed mutations that could destabilize the interaction were obtained. Then, from within a sphere of constant radius from the seed residues, a searched was applied for mutations that suppressed the effect of the seed mutation, e.g., mutations in the opposite chain that were compatible with first mutation and which restabilized the binding interface (FIG. 5 panel A). The stability of the interface was evaluated based on the interface score in REU. FIG. 5 panel B shows representative simulated scores for TCR-Cα. This design strategy was applied to both constant and variant domain interfaces of TCRs and, from over 100,000 models, 108 designs (324 states) were selected for experimental characterization based on the scores and structure models made with Rosetta. To ensure that designs are applicable to a broad set of TCRs, mutations were restricted to the TCR constant domains and the framework region of the variable domains that is highly conserved in most TCRs (ref 24).

176 designs were tested for TCR-C and 72 designs for TCR-V. α and β subunits were co-expressed in Expi293 cells in single wells as MT/MT, MT/WT, and WT/MT states, and the complexes were purified by Ni-affinity chromatography. To compare the expression of each state, SDS-PAGE was performed of equal amounts of protein lysate from the different states. To assess the stability of the α/β interface, thermodynamic properties were measured by nano-differential scanning fluorimetry (nanoDSF). Because strong thermodynamic cooperativity exists between the α/β TCR subunits, nanoDSF provides a single melting peak that corresponds to the Tm of the α/β subunit interface (ref 26, incorporated herein by reference). In addition, the designed TCRs had two disulfide bonds between the α/β interface; nanoDSF results was not affected by the concentration of proteins.

FIG. 6 shows representative designs. For the TCR-C domain (FIG. 6 panels A and B), design 27 (desC27) is a knob-into-hole design. In the WT structure, Arg195 from TCR-Cβ forms a H-bond network at the interface. In the MT/MT state, this Arg195 was mutated to Thr and Ser179 in TCR-Cβ was mutated to Arg which form a new H-bond network and stabilize the interface. In the MT/WT state, two Arg face each other and destabilize the interface. Also, in the WT/MT pair, the interface was underpacked compared with MT/MT.

The design 127 in the constant domain (“desC127”) is a charge-swap design, in which Asp124 and Phe205 in TCR-Ca were changed to Arg and Lys, and Arg in TCR-Cβ was changed to Glu, making a new salt-bridge. In both MT/WT and WT/MT states, charged residues remain and destabilize the interface.

Successful constant designs into a single TCR (“combiC46”). For the TCR-V domain (FIG. 6 panels C and D), design 30 (desV30) is a top performing design. A pair of Gln that interact across the interface were mutated to a Lys and Tyr to form a novel cation-pi interaction. In the MT/WT pair, an unpaired charged residue (Lys) remains at the interface and destabilizes the interaction. The Tm and expression level of MT/WT was significantly decreased as compared with the MT/MT pair.

In addition, studies were performed which confirmed that desV30 can be applied to other TCR-V, such as the DMF4 TCR (ref 42) targeting MART-1 derived peptides (melanocyte-specific antigen). CombiC46 was combined with desV30 to generate “desV30combiC46,” creating a highly orthogonal interface as confirmed by SDS-PAGE and nanoDSF (FIG. 6 panel E). The binding affinity of this TCR (“desV30combiC46”) was equivalent to the starting structure; thus, none of the mutations negatively affected binding affinity (FIG. 6 panel F).

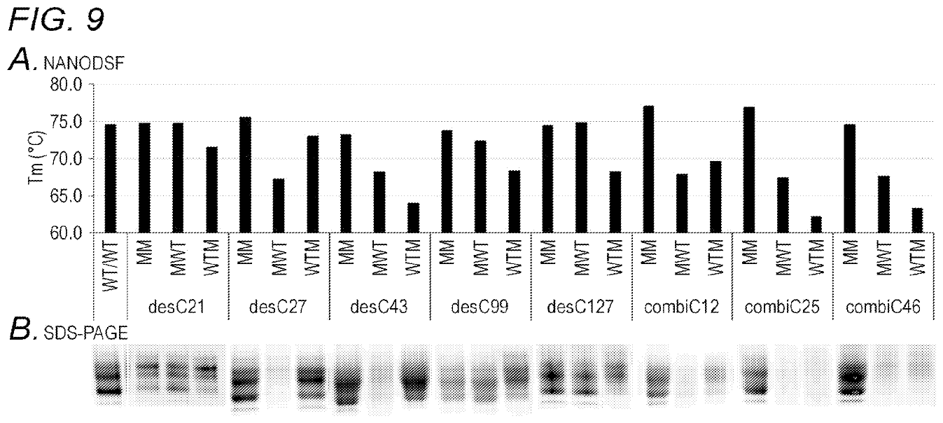

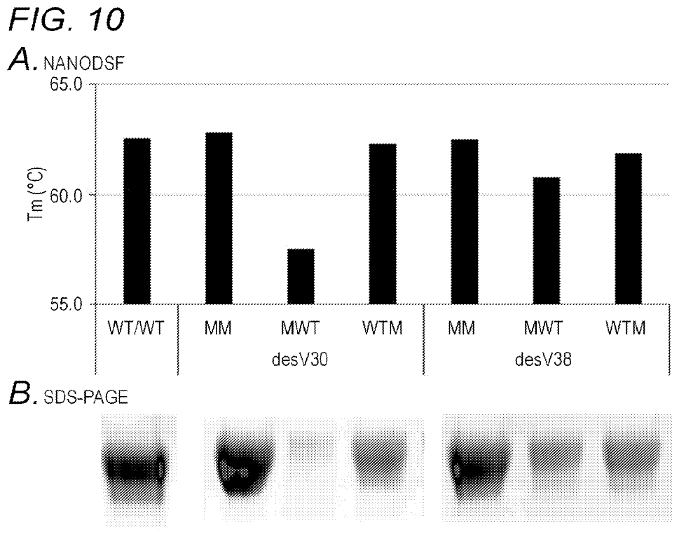

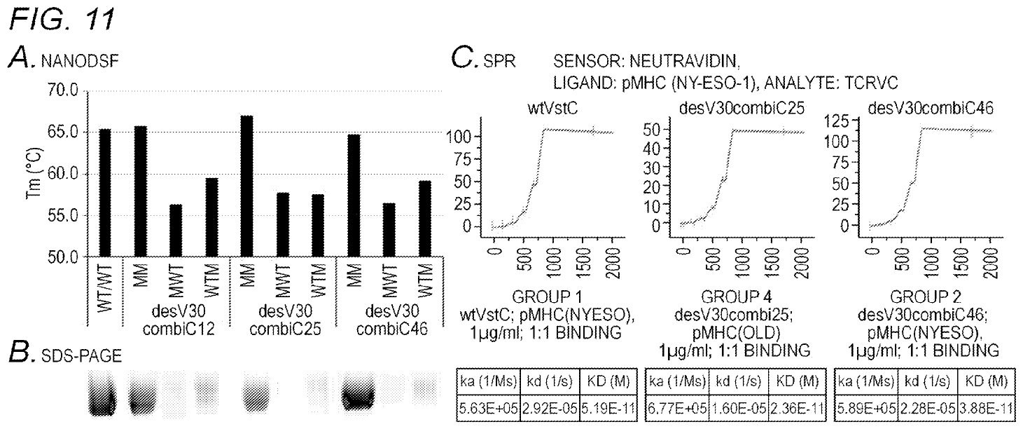

Prepared designs are tabulated in Tables 1-5 and results of design testing is shown in FIGS. 9-11. In FIG. 9, results from testing of TCRC designs is shown. With example combiC25 and combi46 design, the correct pair were well expressed, and mispair (MWT/WTM) were well destabilized. Similarly in FIG. 10, results from TCRV designs is shown. With examples desV30 and desV38 design, the correct pair was well expressed, and mispair (MWT/WTM) was well destabilized. In FIG. 11, results from combined TCRC+TCRV combinative TCR designs is shown. The correct pairs are well expressed, and mispair (MWT/WTM) are well destabilized. Example desV30combiC25 and desV30combi46 are shown to not impair binding affinity.

Further studies to validate the designs of Example 1 are performed. To further validate that the orthogonal TCRs are forming interactions as modeled, X-ray structures of selected orthogonal TCRs are developed. The purified TCR are crystallized by the sitting-drop vapor-diffusion method. Crystals are mixed protein solution with reservoir solution. Drops are seeded with crushed crystals. The plate is checked for two weeks until diffraction-quality crystals are obtained. The crystals are harvested into a drop of reservoir solution and flash frozen in liquid nitrogen. Synchrotron X-ray diffraction data is collected on a single crystal using the SER-CAT Advanced Photon Source. The structures are solved by molecular replacement in PHENIX with the starting TCR structure (PDB: 2F53, incorporated herein by reference). Several rounds of model building and refinement are conducted with Coot and PHENIX software (refs 43 and 44).

### Example 2: Development of Soluble bsTCR-Based Drugs

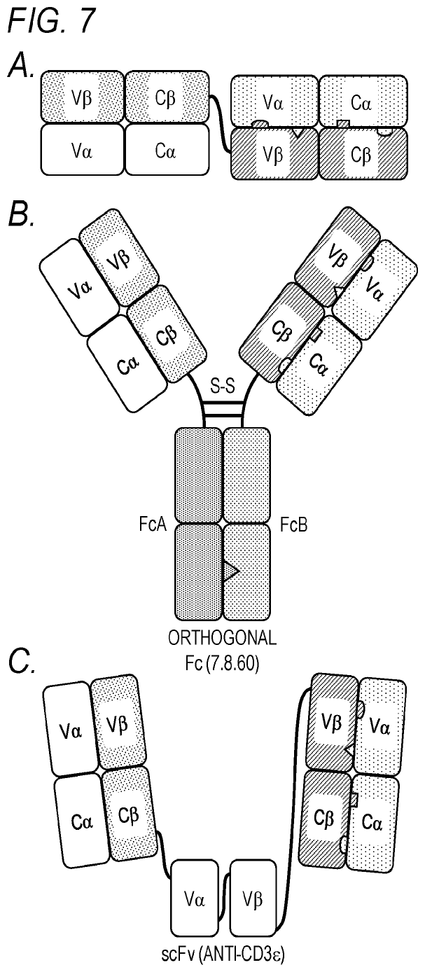

Example soluble bsTCRs are created in the format of (FIG. 7 panel A) Tandem bsTCR, (FIG. 7 panel B) IgG bsTCR, and (FIG. 7 panel C) bsTCR-based trispecific T-cell engager, with in vitro assembly techniques. As a proof-of-concept, a pair of TCRs for NY-ESO-1 and MART-1 peptides on HLA-A02:01, antigens in melanomas, are employed. In this proof-of-concept experiment, linkers are designed to minimize immunogenicity. To achieve precise targeting, the combination of TCR pairs with various affinity are tested. The binding avidities of bsTCRs are evaluated by surface plasmon resonance and fluorescence assisted cell sorting analysis. Drug efficacy is evaluated with in vitro and in vivo tumor cytotoxicity assays.

Tandem bsTCRs are generated by connecting two orthogonal TCRs (“TCR1” and “TCR2”) with a linker designed to minimize immunogenicity (ref 25). The following three domains are co-expressed in Expi293 cells for correct domain assembly: 1) TCR1α, 2) TCR2α, 3) TCR1β-TCR2β (FIG. 7 panel A). A therapeutic feature can be incorporated by conjugating the bsTCRs with cytotoxic payloads by peptide-based lysosomal protease-sensitive linkers, such as those described in references 46 and 47, incorporated herein by reference.

In the form of bsTCRs in an IgG format, the Fc domain of the IgG format activates NK cells and macrophages to exert cytotoxicity. The Fab domains of an antibody are replaced with two orthogonal TCRs (“TCR1” and “TCR2”), which prevent mis-assembly of heavy and light chains. In addition, in the IgG format the homodimeric interface of the Fc domain is modified to a heterodimeric interface to achieve proper heavy chain assembly, such as for example as those described in references 23 and 26, incorporated herein by reference. The IgG bsTCRs are assembled by expressing the following four domains in Expi293 cells (FIG. 7 panel B): 1) TCR1α, 2) TCR2α, 3) TCR1β-Fc-A (e.g., Fc 7.8.60-A), (4) TCR2β-Fc-B (e.g., Fc 7.8.60-B).

TCRs can also be used to redirect endogenous T cells to target tumor cells by fusing an scFv that targets an activating TCR subunit such as CD3ε (refs 50 and 51). The assembly of this format has been challenging due to the subunit mispairing. In other attempts to simplify assembly, prior studies have used only the variable domains of TCRs in a single chain format (scTv), akin to an antibody single chain Fv (scFv). However, scTvs are unstable, aggregation-prone, and poorly soluble, which has prevented creation of this format (ref 37). The orthogonal TCRs addresses these difficulties. A bsTCR-based trispecific T-cell engager is generated by expressing following three domains in Expi293 cells: 1) TCR1α, 2) TCR2α, and 3) TCR1β-scFv (CD3ε)-TCR2β (FIG. 7 panel C).

Plasmids are generated to evaluate expression of these formats. Domain assembly is assessed by SDS-PAGE, size-exclusion chromatography (SEC), and mass spectrometry after purification and removal of glycosylation by PNGaseF. Molecular weight-based analysis (SEC and mass photometry) by tagging domains can be further applied with different molecular weight tags. Binding affinity is measured by SPR and fluorescence-assisted cell sorting.

In vitro and in vivo tumor killing assay with designed soluble bsTCR drugs are performed. For in vitro assays, flow cytometry-based tumor cytotoxicity assays with mixed-population of SK-MEL-5 and M14 melanoma cell lines that express different combinations of antigens and fluorescent markers are performed. Jurkat/NFAT-luc (InvivoGen) is used to assess the efficacy of T-cell engager. For in vivo xenograft tumor cytotoxic analysis, female and male NSG mice (7-9 weeks of age) are injected either subcutaneously (s.c.) or intravenously (i.v.) via tail injection with luciferase-labeled SK-MEL-5 and M14 melanoma cell lines. Seven days after tumor cell injection (day 0) and at days+5 and +12, mice are infused with designed soluble bsTCR drugs (e.g., 1G4 and DMF4 TCR variants). Melanoma tumor cell growth is monitored weekly either with caliper measurement for s.c. tumors, or by bioluminescence using the IVIS kinetic in vivo imaging system (PerkinElmer) for the i.v. metastatic models.

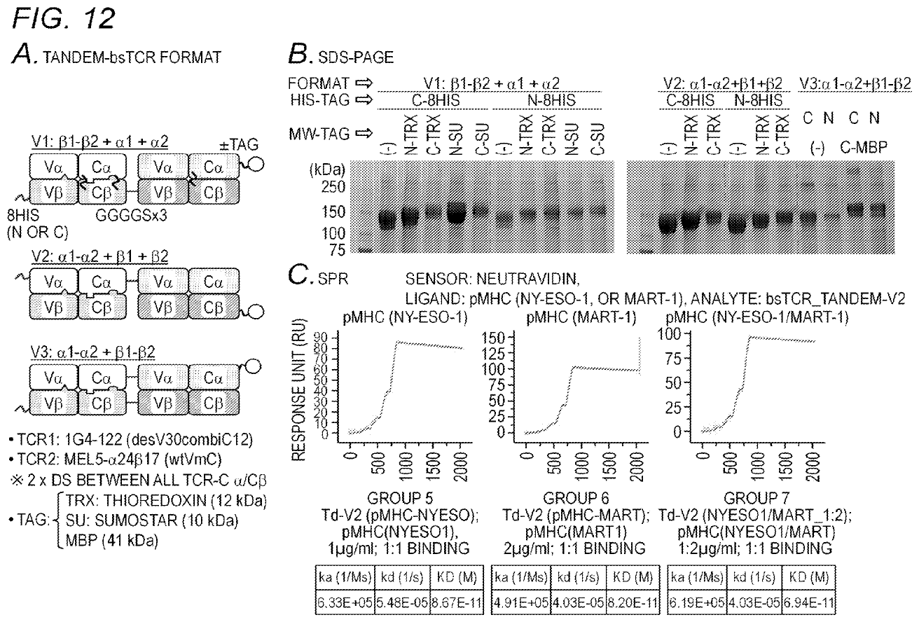

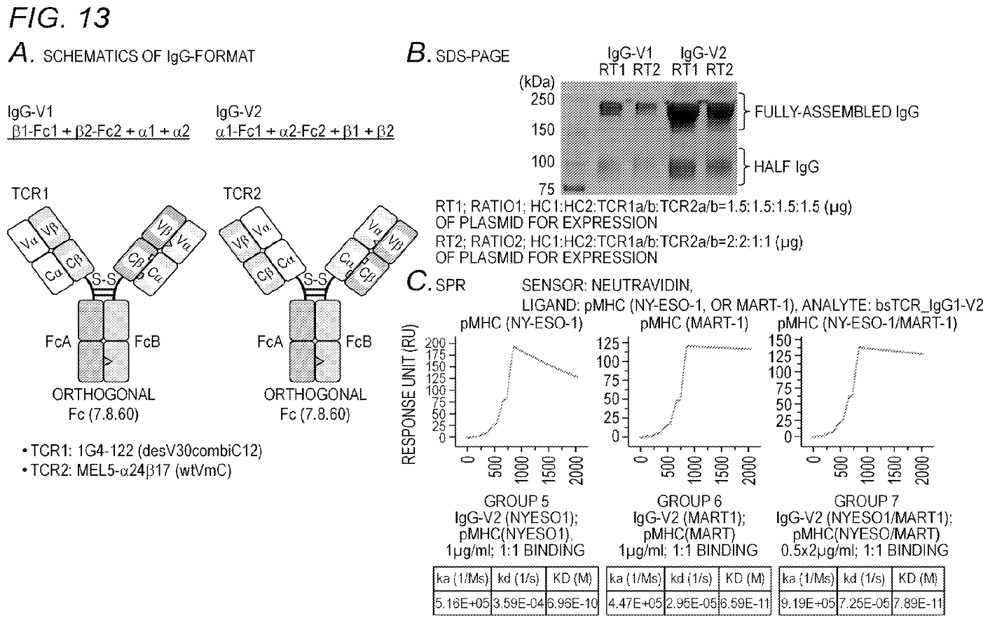

Results from generated bispecific TCR structures is shown in FIGS. 12 and 13. As shown in FIG. 12, tandem designs such as Tandem-v2 bsTCR were well expressed and bound to pMHC presenting NY-ESO-1 or MART-1 peptides. FIG. 13 shows that IgG designs such as IgG-v2 bsTCR were also well expressed and bound to pMHC presenting NY-ESO-1 or MART-1 peptides. Residue numbering and corresponding numbers in other numbering systems used in the art are provided in Tables 6-9 and FIGS. 14A-14B. Alignment of example non-limiting generated TCRCα, TCRCβ, TCRVα, and TCRVβ designs is provided in FIG. 15.

### Methods:

TCRV: 1G4-c49c50 TCR (PDB: 2f53) was used for computational modeling for variant design. All variant design residue in excel sheet was numbered according to this sequence. 1G4-122 TCR was used for experimental testing. Framework regions are same as 1G4-c49c50 so all designs are applicable. Tm and SDS-page results based on this TCR. All variant designs provided based on 1G4-122 TCR. The following are non-limiting TCRs which may be applicable as reference TCRs for numbering and/or backbone structures.
TCR origin (wild type allele): TCRVα (V-Segment: TRAV21*01, J-Segment:
TRAJ6*01), TCRVβ (V-Segment: TRBV6-5*01, J-Segment: TRBJ2-2*01)
1G4 TCR (CDR engineered TCR): PDB 2f54 (doi.org/10.1110/ps.051936406)
1G4-c49c50 TCR (PDB: 2f53 (variant): (doi.org/10.1110/ps.051936406)
1G4-122 TCR (no PDB (doi.org/10.1038/nbt1070) (Table 2b)

TCRC: stTCRC (stC) (disulfate engineered and stabilized TCRC): PDB: 6u07 was used for computational modeling and experimental tests for TCRC designs. All designs are based on this sequence. dsTCRC (dsC) was used for experimental tests for inclusion of constant domain during expression studies because only TCRV does not express well. The following are non-limiting TCRs which may be applicable as reference TCRs for numbering and/or backbone structures.
TCRC origin (wild type allele): UniProt: P01848 (TRAC_HUMAN), UniProt: A0A5B9 (TRBC2_HUMAN)
dsTCRC (dsC) (disulfate engineered TCRC): no PDB (doi.org/10.1038/s41467-020-16231-7)
stTCRC (stC) (disulfate engineered and stabilized TCRC): PDB: 6u07 (doi.org/10.1038/s41467-020-16231-7)

### Example 3: Development of bsTCR CAR-T Therapies

The orthogonal TCRs designed can also be expressed on cells to produce cells that express two different TCRs such as Chimeric antigen receptor (CAR)-T cells. CAR T-cells with engineered scFv from antibodies can perform Boolean logic operations (AND, OR, and NOT) by tuning the avidity of recognition arms or splitting signaling domains. The designed bsTCRs are incorporated into these logic-gated CAR-T cells in the format of (FIG. 3 panel A) Avidity-controlled bsTCR CAR and (FIG. 3 panel B) bsTCR CAR with split signaling domains, with SFG retroviral vector expressing multiple CAR domains by 2A ribosomal skipping sequences. The efficacy of CAR-T cells is evaluated with in vitro cell functional assays and in vivo tumor cytotoxicity assays.

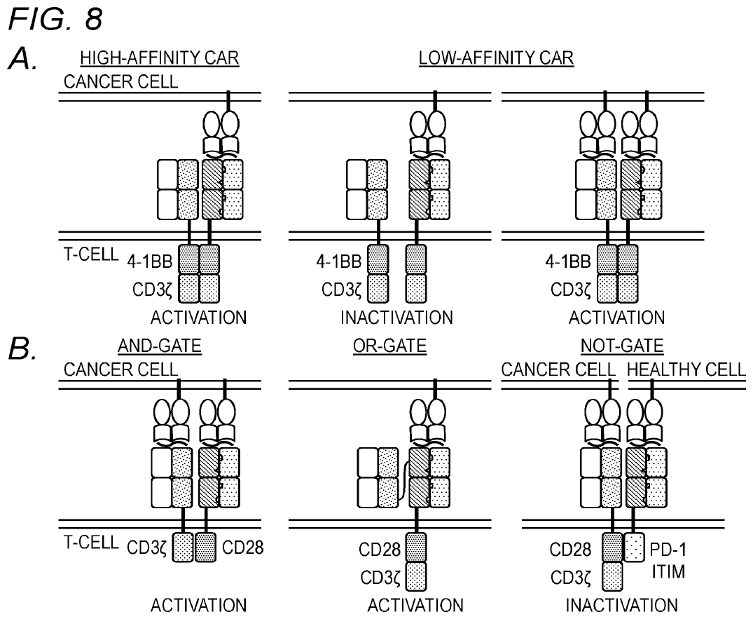

It is possible to control the activity of CAR by tuning the avidity of two recognition domains (refs 13 and 28). CARs with two low-affinity (e.g., μM) binding domains are highly potent only when simultaneously engaging two antigens, which enables AND logic gate control. Conversely, CARs with two high-affinity (e.g., nM) binding domains are activated by recognition of either domain alone, which works as an OR logic gate. Thus, in some embodiments, bsTCRs of this invention are generated into these avidity-controlled CAR-T formats to attain logic gate control of CAR-T with the two different TCRs (FIG. 8 panel A).

Another combinatorial antigen approach is based on segregation of the CD33 domain and the co-stimulatory domain on two different recognition domains (refs 14, 29, 30). As both of these domains are needed for T-cell activation, in this format, CAR-T cells are only activated in response to the engagement of both antigens, which enables AND logic operations. If the CD35 and co-stimulatory domains are linked together to a tandem bsTCR CAR, binding to either antigen will cause T-cell activation, thereby operating as an OR logic gate. If an antigen-binding domain against a ‘healthy tissue’ antigen is fused to inhibitory intracellular signaling domains, such as immunoreceptor tyrosine-based inhibitory motif (ITIM) of programmed cell death protein 1 (PD-1), CAR-T will not work in the presence of the suppressive signal, thus being a NOT logic gate. In some embodiments, bsTCRs of this invention are generated into this split CAR-T format to attain logic control of CAR-T based on the two different TCRs (FIG. 8 panel B).

The stability of each CAR domain and the linker is optimized by Rosetta modeling. The designed bsTCR CAR is expressed with the SFG retroviral vector (refs 55 and 56) in Jurkat T-cell lines and human T cell purchased from the Gulf Coast Regional Blood Center (Houston). To express multiple CAR domains in a single T cell, 2A ribosomal skipping sequences is used, as described in refs 57-59 and incorporated herein by reference. The cell-surface expression of designed bsTCR CAR is assessed by flow cytometry with fluorescent antibodies and pMHC multimers. The functionality of T cells expressing two orthogonal TCRs is assessed with a T-cell proliferation assay, IFN-γ release assay, and flow cytometry-based killing assays with mixed-population of SK-MEL-5 and M14 melanoma cell lines that express different combinations of antigens and fluorescent markers.

In vivo xenograft tumor killing assay of bsTCR CAR-T are performed. Female and male NSG mice (7-9 weeks) are injected either subcutaneously or intravenously (i.v.) via tail injection with luciferase-labeled tumor cells. Seven days after tumor cell injection (day 0) and at days +5 and +12, mice are infused T cells expressing the bsTCR CAR (e.g., 1G4 and DMF4 TCR variants). Melanoma tumor cell growth are monitored weekly either with caliper measurement for s.c. tumors, or by bioluminescence using the IVIS kinetic in vivo imaging system (PerkinElmer) for the i.v. metastatic models.

### Example 4: Further Development of bsTCR Trispecific T-Cell Engager

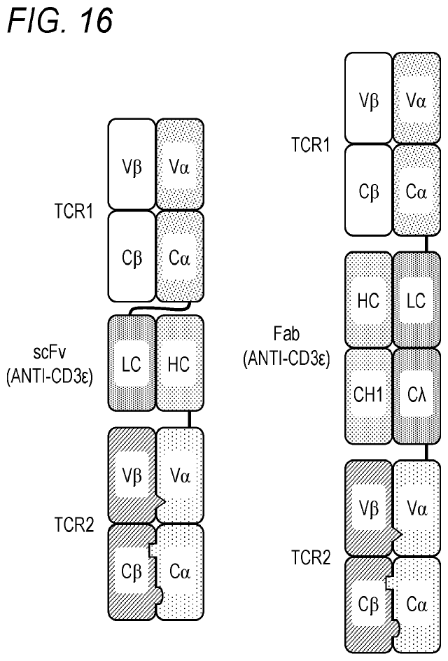

Additional conformations were tested for the trispecific T-cell engager, also referred to as “TriTE” (FIG. 16). As an initial example, a pair of TCRs (“TCR1” as1G4-122 TCR (targeting pMHC with NY-ESO-1 derived peptide) and “TCR2” as MEL5-α24β17 TCR (targeting pMHC with MART-1 derived peptides), were used to redirect human T cells to target tumor cells by fusing an scFv (TriTE1-12) or Fab (TriTE13-24) domains that target an activating TCR subunit such as CD38. Sequences of example configurations are provided in SEQ ID NOs: 51-74 (TriTE1-24); as well as components thereof (SEQ ID NO:75-88).
SEQ ID NO:51 TriTE-main1_TCR1a-TCR2a-imc VLVH
SEQ ID NO:52 TriTE-main2_TCR1a-TCR2a-sp34VLVH
SEQ ID NO:53 TriTE-main3_TCR1a-TCR2a-imc VHVL
SEQ ID NO:54 TriTE-main4_TCR1a-TCR2a-sp34VHVL
SEQ ID NO:55 TriTE-main5_TCR1a-imcVLVH-TCR2a
SEQ ID NO:56 TriTE-main6_TCR1a-sp34VLVH-TCR2a
SEQ ID NO:57 TriTE-main7_TCR1a-imcVHVL-TCR2a
SEQ ID NO:58 TriTE-main8_TCR1a-sp34VHVL-TCR2a
SEQ ID NO:59 TriTE-main9_imcVLVH-TCR1a-TCR2a
SEQ ID NO:60 TriTE-main10_sp34VLVH-TCR1a-TCR2a
SEQ ID NO:61 TriTE-main11 imcVHVL-TCR1a-TCR2a
SEQ ID NO:62 TriTE-main12_sp34VHVL-TCR1a-TCR2a
SEQ ID NO:63 TriTE-main13_TCR1a-TCR2a-LCimc
SEQ ID NO:64 TriTE-main14_TCR1a-TCR2a-LCsp34
SEQ ID NO:65 TriTE-main15_TCR1a-TCR2a-HCimc
SEQ ID NO:66 TriTE-main16_TCR1a-TCR2a-HCsp34
SEQ ID NO:67 TriTE-main17_TCR1a-LCimc-TCR2a
SEQ ID NO:68 TriTE-main18_TCR1a-LCsp34-TCR2a
SEQ ID NO:69 TriTE-main19_TCR1a-HCimc-TCR2a
SEQ ID NO:70 TriTE-main20_TCR1a-HCsp34-TCR2a
SEQ ID NO:71 TriTE-main21_LCimc-TCR1a-TCR2a
SEQ ID NO:72 TriTE-main22_LCsp34-TCR1a-TCR2a
SEQ ID NO:73 TriTE-main23_HCimc-TCR1a-TCR2a
SEQ ID NO:74 TriTE-main24_HCsp34-TCR1a-TCR2a
SEQ ID NO:75 TriTE-sub1_LCimc
SEQ ID NO:76 TriTE-sub2_LCsp34
SEQ ID NO:77 TriTE-sub3_HCimc
SEQ ID NO:78 TriTE-sub4_HCsp34
SEQ ID NO:79 TriTE-sub5_TCR1a
SEQ ID NO:80 TriTE-sub6_TCR1b
SEQ ID NO:81 TriTE-sub7_TCR2a
SEQ ID NO:82 TriTE-sub8_TCR2b
SEQ ID NO:83 anti-CD3_1_imcVLVH
SEQ ID NO:84 anti-CD3_2_sp34VLVH
SEQ ID NO:85 anti-CD3_3_imcVHVL
SEQ ID NO:86 anti-CD3_4_sp34VHVL
SEQ ID NO:87 anti-CD3_5_HCimc
SEQ ID NO:88 anti-CD3_6_HCsp34

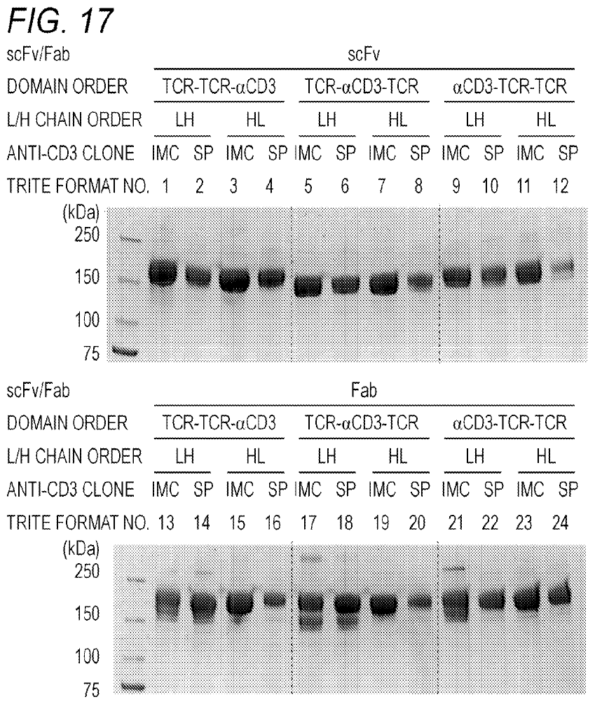

To assemble the bsTCR-based trispecific T-cell engager, the following subunits including two TCRs with designed orthogonal interface (desV30 for the variant domain and combiC46 mutations for the constant domain) have been expressed in Expi293 cells. TriTE1-12:1) TCR1α-scFv (anti-CD3ε)-TCR2α, 2) TCR1β, 3) TCR2β, TriTE13-24:1) TCR1α-LC or HC (anti-CD3ε)-TCR2α, 2) TCR1β, 3) TCR2β, (4) LC or HC (anti-CD3ε). The assembled TriTE proteins were purified with Ni-affinity chromatography use equal amounts of protein lysate. Correct construction and size was determined by SDS-PAGE, as shown in FIG. 17.

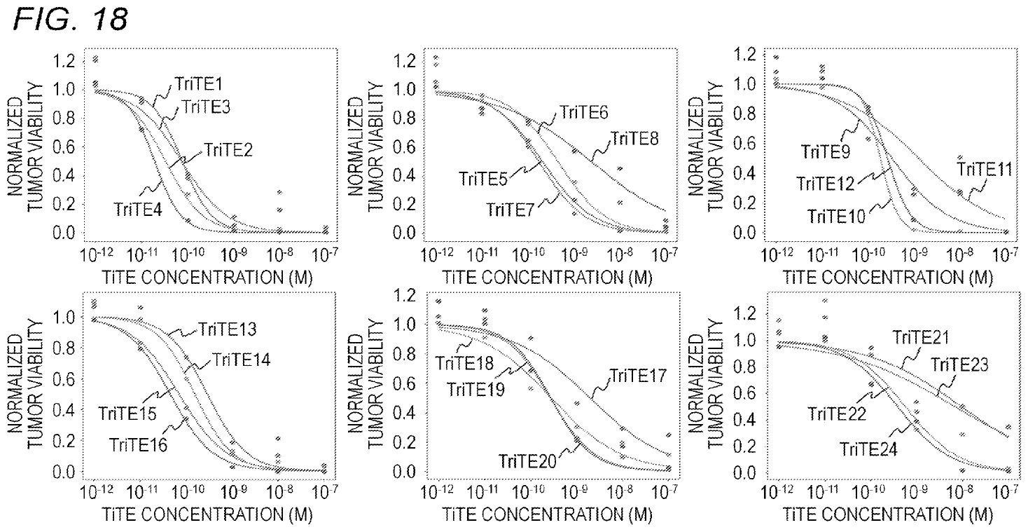

T-cell redirected tumor lysis assays using exogenous peptides were performed as follows (FIGS. 18-20). Human primary CD3+ T cells (StemExpress, catalog number: PB03020C, lot number: 2111150142, Donor number: D001006110) were thawed and resuspended in complete RPMI medium (RPMI 1640, 10% FBS, 1% Penicillin-Streptomycin, 1% GlutaMAX) containing anti-CD28 antibody (BD Biosciences 555725, CD28.27, final concentration: 2.5 μg/mL) and human interleukin-2 (R&D systems 202-IL/CF, final concentration: 2 ng/mL) and expanded in the flasks which is precoated overnight with anti-CD3 antibody (BD Biosciences cat #555329, UCHT1, pre-coat concentration: 5 μg/mL). T cell expansion was allowed between 4 and 14 days before use. At day 7, T cells were re-stimulated with complete RPMI including anti-CD28 antibody and IL-2 in a new flask precoated with anti-CD3 antibody. At the day before the killing assay, the tumor cells were removed from the culture flasks using TrypLE™ Express Enzyme (Gibco #12605010) and resuspended in complete medium and seeded at 5000 cells/well in CELLSTAR® 96, clear bottom plate (Greiner Bio-One #655098) and incubated overnight at 5% CO2 and 37° C. The next day, the medium was removed and SLLMWITQC (NY-ESO-1 derived) peptide and/or ELAGIGILTV (MART-1 derived) peptide were mixed with pure RPMI (RPMI 1640), added to the well at 1 μM (total volume of 100 μL RPMI including final 0.01% dimethyl sulfoxide), and incubated at 5% CO2 and 37° C. for three to six hours. Then, 100 μL of peptide solution was removed from each well and bsTCR-TriTE were mixed in complete RPMI medium (RPMI 1640, 10% FBS, 1% P/S, 1% GlutaMAX) and added to the well (50 μL/well, 2× indicated TriTE concentration). The expanded primary T cells were washed once with pure RPMI, resuspended in complete RPMI, and then 50 μL of resuspended solution were added to the well at 50 K cells/well (total 100 μL, 1× indicated TriTE concentration). Non-peptide plates were treated similarly to those with peptide, except that no peptide was used in the procedure (final total volume 100 μL of RPMI 1640 including 10% FBS, 1% Penicillin-Streptomycin, 1% GlutaMAX, 0.01% dimethyl sulfoxide, 1× indicated TriTE concentration). The cells were incubated at 5% CO2 and 37° C. for 48 h. After 48 h, the plates were washed twice gently with pure RPMI. To determine the amount of tumor cells that were alive at the end of the incubation, 100 μL of pure RPMI1640 and 100 μL of CellTiter-Glo® 2.0 (Promega: #G9242) were added, mixed with a shaker at 500 rpm for 2 minutes, and incubated in the dark for 10 minutes. Finally, luminescence was read with a CLARIOstar Plus microplate reader (BMG LABTECH). Materials included: Primary T cells (StemCell cat #PB03020C, lot #2111150142, Donor #D002006110); Tumor cells (adherent cells); anti-CD3 (BD Biosciences 555329, UCHT1, final conc: 5 μg/mL)> for precoat; anti-CD28 (BD Biosciences 555725, CD28.27, final conc: 2.5 g/mL); Interleukin-2 (R&D systems 202-IL/CF, final conc: 2 ng/mL); RPMI 1640 (pure: add noting, complete: 10% FBS, 1% P/S, 1% GlutaMAX); Fetal Bovine Serum (Omega Scientific #FB11A): Penicillin-Streptomycin (10,000 U/mL) (Life Technologies Inc. #15140122); TrypLE™ Express Enzyme (Gibco #12605010); DPBS (Life Technologies #14190144); GLUTAMAX I, 100× (TCF/Life Technologies Inc. #35050061); CELLSTAR® 96, clear bottom plate (Greiner Bio-One #655098); CellTiter-Glo® 2.0 (Promega: #G9242). Data is shown in FIG. 18.

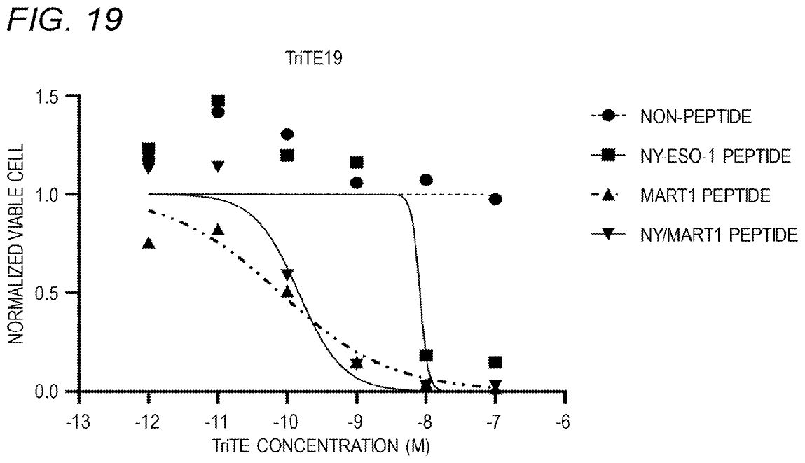

FIG. 19 shows one exemplar plot focusing on TriTE19. T-cell redirected tumor lysis assays using exogenous peptides were performed as described above except that the A375 tumor cells were treat with 4 different peptide condition, non-peptide, 1 μM of SLLMWITQC (NY-ESO-1 derived; SEQ ID NO:47) peptide only, 1 μM of ELAGIGILTV (MART-1 derived peptide; SEQ ID NO:48) only, or both 1 μM of SLLMWITQC (SEQ ID NO:47) and 1 μM of ELAGIGILTV (SEQ ID NO:48) peptides.

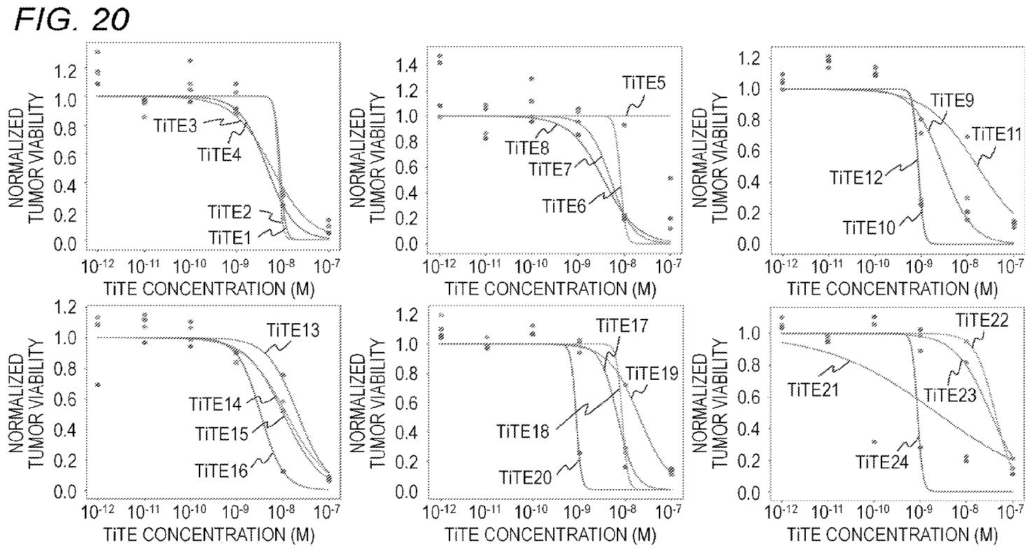

FIG. 20 shows T-cell redirected tumor lysis assays using exogenous peptides, which were performed as described above except using the SK-MEL-5 cells without peptide treatment.

### Example 5: Use of bsTCR Trispecific T-Cell Engager

To assemble the bsTCR-based trispecific T-cell engager (bsTCR-TriTE), the following subunits including two TCRs with the designed orthogonal interface (desV30 for the variant domain and combiC46 mutations for the constant domain) are expressed in Expi293 cells. TriTE1-12:1) TCR1α-scFv (anti-CD3ε)-TCR2α, 2) TCR1β, 3) TCR2β, TriTE13-24:1) TCR1α-LC or HC (anti-CD3ε)-TCR2α, 2) TCR1β, 3) TCR2β, (4) LC or HC (anti-CD3ε). The assembled TriTE proteins will be purified with Ni-affinity chromatography and size exclusion chromatography.

For in vivo xenograft tumor cytotoxic analysis, female and male NOD-SCID-Il2rg^{-/-} (NSG) mice (7-9 weeks of age) are anesthetized and injected either subcutaneously (s.c.) or intravenously (i.v.) via tail injection with 1×10⁷ human T cells and 1×10⁶ of luciferase-labeled tumor cell lines. Seven days after tumor cell injection (day 0) and at days +5 and +12, mice are infused with TriTE proteins intraperitoneally (i.p.) or intravenously (i.v.) at the specified infusion rates using sterile surgical technique. The tumor cell growth is monitored weekly either with caliper measurement for s.c. tumors, or by bioluminescence using the IVIS kinetic in vivo imaging system (PerkinElmer) for the i.v. metastatic models.

### Example 6: Further Development and Use of bsTCR-CAR Constructs

A bsTCR CAR targeting MART-1 or gp100 peptides on HLA-A02:01 is designed to compose the following domains that target the cell membrane with signal peptide (SP) and transmembrane domain (TM): (1) SP1-TCR1β, (2) SP2-TCR2β, (3) SP3-TCR1α-linker-TCR2α-Flag-hinge-CD8aTM-CD28-CD3z. To express multiple domains in a single T cell, 2A ribosomal skipping sequences are used. The CARs are expressed in HEK293T cells and assessed the cell-surface expression by flow cytometry (FCM) with anti-FLAG antibodies (Biolegend) and pMHC pentamers (Proimmune). The bsTCR CAR are transduced into T-cells with the SFG retroviral vector and expression on human T cell is confirmed from peripheral blood mononuclear cells. The functionality of bsTCR CAR-T is assessed with a T-cell proliferation assay, cytokine release assay (INF-γ, IL-2), and cancer-killing assays with peptide-pulsed T2 cells and/or untreated melanoma cell lines that express different combinations of antigens.

For in vitro assays, flow cytometry-based tumor cytotoxicity assays with mixed-population of melanoma cell lines that express different combinations of antigens and fluorescent markers are performed. For in vivo xenograft tumor cytotoxic analysis, female and male NOD-SCID-Il2rg^{-/-} (NSG) mice (7-9 weeks of age) are injected either subcutaneously (s.c.) or intravenously (i.v.) via tail injection with 1×10⁷ bsTCR-CAR-T cells and 1×10⁶ of luciferase-labeled melanoma cell lines. Seven days after tumor cell injection (day 0) and at days +5 and +12, mice are infused designed bsTCR-CAR. Melanoma tumor cell growth is monitored weekly either with caliper measurement for s.c. tumors, or by bioluminescence using the IVIS kinetic in vivo imaging system (PerkinElmer) for the i.v. metastatic models.

TABLE 1
Characterization of example designed TCR constant (TCRC) proteins.

| | | | | | | | Relative | | |
|---|---|---|---|---|---|---|---|---|---|
| | | Mutation (numbering per | | Tm (° C.) | | | yield (/MM) | | |
| Design (TCR | | reference PDB: 6U07) | | MM | | | MM | | |
| constant) | design type | chain A (chA) | chain B (chB) | (WTWT) | MWT | WTM | (WTWT) | MWT | WTM |
| stTCRC | (no design) | NA | NA | 74.4° C. | NA | NA | 1.00 | NA | NA |
| desC20 | charge to | chA D124F F205W | chB R139L | 75.3° C. | 75.9° C. | 71.5° C. | 1.00 | 1.38 | 0.65 |
| | hydrophobic | | | | | | | | |
| desC21 | other | chA D124Q F205K | chB R139L | 74.7° C. | 74.7° C. | 71.5° C. | 1.00 | 1.23 | 1.00 |
| desC27 | knob-into-hole | chA S179R | chB R195T | 75.6° C. | 67.2° C. | 73.0° C. | 1.00 | 0.17 | 0.79 |
| desC43 | knob-into-hole | chA S179R | chB R195S | 75.0° C. | 67.6° C. | 69.9° C. | 1.00 | 0.14 | 0.84 |
| desC56 | charge swap | chA R171Q S172D | chB D170K | 74.8° C. | 76.1° C. | 71.8° C. | 1.00 | 1.02 | 0.83 |
| desC99 | charge swap | chA F175R S177K | chB K142E R197S | 73.7° C. | 72.3° C. | 68.4° C. | 1.00 | 1.21 | 1.05 |
| desC100 | charge swap | chA D145H F175R | chB K142E R197S | 72.9° C. | 69.0° C. | 68.4° C. | 1.00 | 0.86 | 1.34 |
| | | S177K | | | | | | | |
| desC127 | charge swap | chA D124R F205K | chB R139E | 74.5° C. | 74.8° C. | 68.3° C. | 1.00 | 0.75 | 0.59 |
| desC128 | charge swap | chA D124R F205K | chB R139D | 73.8° C. | 74.8° C. | 66.3° C. | 1.00 | 1.06 | 0.80 |
| combiC12 | combination | chA D124Q S179R | chB R139L R195T | 77.2° C. | 68.1° C. | 69.5° C. | 1.00 | 0.15 | 0.43 |
| (desC21_C27) | | F205K | | | | | | | |
| combiC25 | combination | chA D124Q F175R | chB R139L K142E | 76.9° C. | 67.4° C. | 62.2° C. | 1.00 | 0.11 | 0.18 |
| (desC21_27_99) | | S177K S179R F205K | R195T R197S | | | | | | |
| combiC26 | combination | chA D124Q D145H | chB R139L K142E | 76.1° C. | 65.5° C. | 62.1° C. | 1.00 | 0.11 | 0.25 |
| (desC21_27_100) | | F175R S177K S179R | R195T R197S | | | | | | |
| | | F205K | | | | | | | |
| combiC46 | combination | chA D124R S179R | chB R139E R195S | 74.5° C. | 67.6° C. | 63.3° C. | 1.00 | 0.23 | 0.24 |
| (desC43_127) | | F205K | | | | | | | |
| combiC48 | combination | chA D124R S179R | chB R139D R195S | 73.9° C. | 67.6° C. | 61.3° C. | 1.00 | 0.37 | 0.41 |
| (desC43_128) | | F205K | | | | | | | |

Legend: TCRC: TCR constant domain; desC: TCRC design; combiC: combinative desC; Tm; melting temperature determined by nanoDSF; MM: mutant-mutant pair; MWT: mutant-wildtype pair; WTM: wildtype-mutant pair. stTCRC: disulfate engineered and stabilized TCRC PDB: 6u07 (rcsb.org/sequence/6u07; doi.org/10.1038/s41467-020-16231-7)

TABLE 2
Characterization of example designed TCR variant (TCRV) proteins.

| | | Mutation (numbering per | | | | | | | |
|---|---|---|---|---|---|---|---|---|---|
| | | reference PDB: 2F53) | | | Tm | | | relative | |
| Design (TCR | | chain A | chain B | MM | (° C.) | | MM | yield (/MM) | |
| variant) | design type | (chA) | (chB) | (WTWT) | MWT | WTM | (WTWT) | MWT | WTM |
| TCR_wtVdsC | (no design) | NA | NA | 62.5° C. | NA | NA | 1.00 | NA | NA |
| desV11dsC | polar to cation-pi | chA Q37Y | chB Q35K | 61.3° C. | 60.5° C. | 58.8° C. | 1.00 | 0.58 | 0.25 |
| desV30dsC | polar to cation-pi | chA Q37K | chB Q35Y | 62.8° C. | 57.5° C. | 62.3° C. | 1.00 | 0.08 | 0.39 |
| desV31dsC | polar to charge | chA Q37K | chB Q35D | 63.8° C. | 57.2° C. | 61.6° C. | 1.00 | 0.40 | 0.55 |
| desV32dsC | polar to charge | chA Q37D | chB Q35K | 61.6° C. | 58.9° C. | 58.6° C. | 1.00 | 0.61 | 0.34 |
| desV38dsC | polar to | chA Q37L | chB Q35M | 62.5° C. | 60.7° C. | 61.8° C. | 1.00 | 0.43 | 0.54 |
| | hydrophobic | | | | | | | | |
| des V40dsC | polar to | chA Q37V | chB Q35M | 61.8° C. | 59.3° C. | 62.0° C. | 1.00 | 0.13 | 0.45 |
| | hydrophobic | | | | | | | | |
| des V58dsC | new salt bridge | chA S108K | chB G38E | 62.4° C. | 63.6° C. | 61.2° C. | 1.00 | 0.82 | 0.22 |

Legend: desV: TCR variant domain design; wtVdsC: wildtype TCR variant domain and TCR constant domain with optional additional disulfide bond; Tm: melting temperature determined by nanoDSF; MM: mutant-mutant pair; MWT: mutant-wildtype pair; WTM: wildtype-mutant pair. dsTCRC(dsC): disulfate engineered TCRC (doi.org/10.1038/s41467-020-16231-7).

TABLE 3
Characterization of example designed TCRVC (variant and constant) combinative proteins.

| | | | Mutation (TCRC) (numbering | | | | | | | |
|---|---|---|---|---|---|---|---|---|---|---|
| Design (TCR | Mutation (TCRV) (numbering | | per reference PDB: 6U07) | | Tm (° C.) | | | relative yield (/MM) | | |
| variant + | per reference PDB: 2F53) | | chain A | chain B | MM | | | MM | | |
| constant) | chain A | chain B | (chA) | (chB) | (WTWT) | MWT | WTM | (WTWT) | MWT | WTM |
| TCR_wtVstC | NA | NA | NA | NA | 65.4° C. | NA | NA | 1.00 | NA | NA |
| desV30combiC12 | chA Q37K | chB Q35Y | chA D124Q | chB | 65.7° C. | 56.2° C. | 59.6° C. | 1.00 | 0.15 | 0.29 |
| (desV30, | | | S179R | R139L | | | | | | |
| desC21_C27) | | | F205K | R195T | | | | | | |
| desV30combiC25 | chA Q37K | chB Q35Y | chA D124Q | chB | 67.0° C. | ND | 57.5° C. | 1.00 | ND | 0.12 |
| (desV30, | | | F175R | R139L | | | | | | |
| desC27_21_99) | | | S177K | K142E | | | | | | |
| | | | S179R | R195T | | | | | | |
| | | | F205K | R197S | | | | | | |
| des V30combiC26 | chA Q37K | chB Q35Y | chA D124Q | chB | 65.3° C. | ND | 57.2° C. | 1.00 | ND | 0.14 |
| (desV30, | | | D145H | R139L | | | | | | |
| desC27_21_100) | | | F175R | K142E | | | | | | |
| | | | S177K | R195T | | | | | | |
| | | | S179R | R197S | | | | | | |
| | | | F205K | | | | | | | |
| desV30combiC46 | chA Q37K | chB Q35Y | chA D124R | chB | 65.3° C. | ND | 59.1° C. | 1.00 | ND | 0.15 |
| (desV30, | | | S179R | R139E | | | | | | |
| desC43_127) | | | F205K | R195S | | | | | | |
| desV30combiC48 | chA Q37K | chB Q35Y | chA D124R | chB | 64.4° C. | ND | 59.6° C. | 1.00 | ND | 0.08 |
| (desV30, | | | S179R | R139D | | | | | | |
| desC43_128) | | | F205K | R195S | | | | | | |

Legend: desV: TCR variant domain design; wtVdsC: wildtype TCR variant domain and TCR constant domain with optional additional disulfide bond; Tm: melting temperature determined by nanoDSF; MM: mutant-mutant pair; MWT: mutant-wildtype pair; WTM: wildtype-mutant pair.

TABLE 4
Additional example constant designs (desC).

| | Final mutation_chA | Final mutation_chB |
|---|---|---|
| | (numbering per | (numbering per |
| Design | reference PDB: 6U07) | reference PDB: 6U07) |
| des1 | chA Y128F T144M D145F L168A | chB K142L R197I |
| | F175L S177L | |
| des2 | chA T144M L168A F175K S177M | chB T140K K142D R197I |
| des3 | chA T144M D145N F175R S177L | chB T140Y K142E R197T |
| des4 | chA Y128F D145M L168I F175I | chB K142Q R197M |
| | S177V | |
| des5 | chA Y128F D145K L168I F175V | chB K142E R197M |
| | S177I | |
| des6 | chA D124R F205E | chB R139D |
| des7 | chA D124E F205K | chB R139D |
| des8 | chA D124Q F205K | chB R139D |
| des9 | chA V140T V181L W183K | chB V146A L148W |
| des10 | chA V140M L142M V181A | chB V146G L148F |
| des11 | chA S1791 V181Q | chB D175Q R195A |
| des12 | chA K134E | chB E240K |
| des13 | chA F175K | chB K142D |
| des14 | chA S179A | chB R195M |
| des15 | chA K134E | chB E126R |
| des16 | chA T144M D145N F175R S177L | chB E136Q T140Y K142E R197T |
| des17 | chA T144E D145N F175R S177K | chB E136Q T140Y K142E R197S |
| des18 | chA D145H F175R S177Q | chB T140Y K142E R197T |
| des19 | chA S177H | chB K142W R197T |
| des20 | chA D124F F205W | chB R139L |
| des21 | chA D124Q F205K | chB R139L |
| des22 | chA V140M L142M V181A W183T | chB F130Y V146G L148W |
| des23 | chA V140T L142S V181L W183K | chB F130Y V146A L148W |
| des24 | chA T163G V181R | chB D175L R195A |
| des25 | chA Y128H V140A L142S V181M | chB V146A L148F |
| des26 | chA V140T V181A W183R | chB L148W |
| des27 | chA S179R | chB R195T |
| des28 | chA S179I V181I | chB R195A |
| des29 | chA S179L V181I | chB D175M R195A |
| des30 | chA S179I V181I | chB D175E R195A |
| des31 | chA W183S | chB L148A S193Y |
| des32 | chA V181M W183Q | chB L148W D175I R195A |
| des33 | chA V140L W183G | chB L148A D175I S193Y R195M |
| des34 | chA T163G V181L | chB V146A S193F R195L |
| des35 | chA V181Q W183K | chB L148W D175V R195A |
| des36 | chA F205R P207G | chB A135E |
| des37 | chA F205K P207G | chB A135E |
| des38 | chA L130A V181Q | chB V146Q |
| des39 | chA Y161V | chB L179M E181Y |
| des40 | chA Y161I | chB L179M E181H |
| des41 | chA S179R | chB R195G |
| des42 | chA S179R | chB R195A |
| des43 | chA S179R | chB R195S |
| des44 | chA T163G S179I V181I | chB D175L R195A |
| des45 | chA T163G S179I V181I | chB D175L S193A R195A |
| des46 | chA T163M S179L | chB D175A S193I R195T |
| des47 | chA S133H | chB |
| des48 | chA S133V | chB |
| des49 | chA R171P S172W | chB D170A |
| des50 | chA R171D S172W | chB D170K |
| des51 | chA D132L | chB A128G |
| des52 | chA D132A K138L | chB A128I |
| des53 | chA D132S K138L | chB A128I |
| des54 | chA D132L K138R | chB A128G |
| des55 | chA R171E S172D | chB D170K |
| des56 | chA R171Q S172D | chB D170K |
| des57 | chA R171E S172D | chB D170R |
| des58 | chA D164N | chB Q177E |
| des59 | chA D164N K165T | chB Q177E |
| des60 | chA Y161V | chB L179M E181H |
| des61 | chA Y161V | chB L179M E181F |
| des62 | chA Y161V | chB E181H Q182Y |
| des63 | chA Y161I | chB E181H Q182Y |
| des64 | chA Y161V | chB E181W Q182Y |
| des65 | chA F175R S177M | chB K142T R197T |
| des66 | chA T144M F175D S177L | chB R197S |
| des67 | chA T144M D145N F175K S177L | chB K142D R197T |
| des68 | chA T144E F175K S177L | chB E136Q K142D R197T |
| des69 | chA F175R S177L | chB K142T R197T |
| des70 | chA T144M F175D S177L | chB R197V |
| des71 | chA S177H | chB K142W T144I R197T |
| des72 | chA S177T | chB K142Y T144I R197G |
| des73 | chA S177A | chB K142L R197T |
| des74 | chA L168I F175V S177Q | chB K142Q R197M |
| des75 | chA D124R F205E | chB R139N |
| des76 | chA F205K | chB R139D |
| des77 | chA D124A F205R | chB R139D |
| des78 | chA D124W F205W | chB R139L |
| des79 | chA V181Q | chB D175Q R195A |
| des80 | chA A126Q Y128V F205K | chB R139L |
| des81 | chA Y128M F205K | chB R139L T140Y |
| des82 | chA S133R | chB |
| des83 | chA R171P S172W | chB D170A |
| des84 | chA R171E S172D | chB D170K |
| des85 | chA P207G | chB S133T A135Q |
| des86 | chA P207G | chB S133T A135E |
| des87 | chA F205R P207G | chB A135Q |
| des88 | chA F205R P207G | chB A135D |
| des89 | chA S133E | chB E131A |
| des90 | chA K134E | chB E240R |
| des91 | chA K134D | chB E240K |
| des92 | chA K138E | chB E126R |
| des93 | chA D124T chA F205K | chB R139D |
| des94 | chA F205K | chB R139E |
| des95 | chA F175E chA S177E | chB R197V |
| des96 | chA F175R S177Q | chB T140K K142E R197T |
| des97 | chA F175K S177L | chB K142D R197T |
| des98 | chA F175R S177L | chB K142E R197T |
| des99 | chA F175R S177K | chB K142E R197S |
| des100 | chA D145H F175R S177K | chB K142E R197S |
| des101 | chA ASP 124 PHE | chB R139D |
| des102 | chA ASP 124 TRP | chB R139D |
| des103 | chA D124F F205W | chB R139D |
| des104 | chA D124W F205W | chB R139D |
| des105 | chA ASP 124 LYS | chB R139D |
| des106 | chA ASP 124 ARG | chB R139D |
| des107 | chA ASP 124 TYR | chB R139D |
| des108 | chA D124Y F205K | chB R139D |
| des109 | chA ASP 124 LYS | chB R139E |
| des110 | chA ASP 124 ARG | chB R139E |
| des111 | chA ASP 124 TRP | chB R139E |
| des112 | chA D124S F205R | chB R139E |
| des113 | chA: SER 158 ARG | chB: ALA 184 ASP |
| des114 | chA: SER 158 ARG | chB: ALA 184 ASP chB: LEU 185 |
| | | ARG |
| des115 | chA: SER 158 ASP | chB: ALA 184 LYS |
| des116 | chA: SER 158 GLU | chB: ALA 184 LYS |
| des117 | chA: SER 158 TYR | chB: ALA 184 LYS |
| des118 | chA: SER 158 TYR | chB: ALA 184 GLU |
| des119 | chA: SER 158 TRP | chB: LEU 185 ARG |
| des120 | chA: SER 158 TRP | chB: LEU 185 LYS |
| des121 | chA: SER 172 ARG | chB: PHE 202 GLU |
| des122 | chA: SER 172 GLU | chB: PHE 202 ARG |
| des123 | chA: PHE 175 ARG | chB: LYS 142 GLU |
| des124 | chA: MET 173 TYR chA: PHE 175 | chB: LYS 142 ASP |
| | LYS | |
| des125 | chA D124R F205R | chB R139E |
| des126 | chA D124R F205H | chB R139E |
| des127 | chA D124R F205K | chB R139E |
| des128 | chA D124R F205K | chB R139D |

TABLE 5
Additional example variant designs (desV).

| | Final mutation_chA | Final mutation_chB |
|---|---|---|
| | (numbering per | (numbering per |
| Design | reference PDB: 2F53) | reference PDB: 2F53) |
| desV1 | chA K41D | chB E104R |
| desV2 | chA Q37E | chB Q35K |
| desV3 | chA R105E | chB M39R |
| desV4 | chA F103E | chB L41G F102Y |
| desV5 | chA K41Q | chB R107K |
| desV6 | chA K41R G42A | chB R107K |
| desV7 | chA K41D | chB E104S |
| desV8 | chA K41Q | chB |
| desV9 | chA Q37N | chB Q35R |
| desV10 | chA Q37R | chB Q35S |
| desV11 | chA Q37Y | chB Q35K |
| desV12 | chA Q37Y | chB Q35R |
| desV13 | chA R105D | chB M39T |
| desV14 | chA R105S | chB M39T |
| desV15 | chA R105D | chB M39K |
| desV16 | chA R105E | chB M39S |
| desV17 | chA P101W F103M | chB Y33W |
| desV18 | chA P101Y F103S | chB Y33A L41I |
| desV19 | chA I100L | chB L43T |
| desV20 | chA I100L | chB L43S |
| desV21 | chA I100L | chB Y29F L43A |
| desV22 | chA I100L | chB Y29W L43T |
| desV23 | chA N31L L48V | chB E99T |
| desV24 | chA L48S | chB E99T |
| desV25 | chA S45L L48S | chB E99T |
| desV26 | chA L48F | chB E99F |
| desV27 | chA F35A | chB F102W |
| desV28 | chA F35N | chB F102W |
| desV29 | chA Q37K | chB Q35E |
| desV30 | chA Q37K | chB Q35Y |
| desV31 | chA Q37K | chB Q35D |
| desV32 | chA Q37D | chB Q35K |
| desV33 | chA Q37R | chB Q35T |
| desV34 | chA Q37T | chB Q35R |
| desV35 | chA Q37R | chB Q35A |
| desV36 | chA Q37A | chB Q35R |
| desV37 | chA Q37I | chB Q35M |
| desV38 | chA Q37L | chB Q35M |
| desV39 | chA Q37S | chB Q35R |
| desV40 | chA Q37V | chB Q35M |
| desV41 | chA K41D | chB E104D |
| desV42 | chA K41D | chB E104K |
| desV43 | chA K41D | chB E104T |
| desV44 | chA K41D | chB E104V |
| desV45 | chA K41Q | chB F8Y |
| desV46 | chA K41Q | chB K7R |
| desV47 | chA K41T | chB V86L |
| desV48 | chA K41T | chB V86W |
| desV49 | chA L43A | chB L41F |
| desV50 | chA L43F | chB L41A |
| desV51 | chA Q37V L43A | chB Q35R L41M |
| desV52 | chA Q37V L43A | chB L41M |
| desV53 | chA: GLY40GLU | chB: ARG107LYS |
| desV54 | chA: GLY40LYS | chB: ARG107PHE |
| desV55 | chA: PHE103TRP | chB: ARG42ILE |
| desV56 | chA: PHE103TYR | chB: SER31ALA |
| desV57 | chA: SER108LYS | chB: GLY38ASP |
| desV58 | chA: SER108LYS | chB: GLY38GLU |
| desV59 | chA: ARG105ASP | chB: MET39ASN |
| desV60 | chA: ARG105ASP | chB: MET39ARG |
| desV61 | chA: THR86LEU | chB: MET39TRP |
| desV62 | chA: THR86VAL | chB: MET39TYR |
| desV63 | chA: SER45LYS | chB: PHE101ASP |
| desV64 | chA: SER45PRO | chB: PHE101VAL |
| desV65 | chA: GLY40MET | chB: ARG107PHE: ASP150PRO |
| desV66 | chA: GLY40ASN | chB: ARG107PHE: ASP150PRO |
| desV67 | chA: GLY40ARG | chB: ARG107PHE: ASP150PRO |
| desV68 | chA: GLY40TRP | chB: ARG107ILE: ASP150PRO |
| desV69 | chA: ARG105ASP | chB: MET39LYS: ARG42ILE |
| desV70 | chA: ARG105ASP | chB: GLY38ASP: MET39LYS |
| desV71 | chA: ARG105ASP | chB: GLY38ASN: MET39LYS |
| desV72 | chA: ARG105ASP | chB: MET39ARG: ARG42ILE |
| desV73 | chA Q37R | chB Q35Y |

TABLE 6
Alignment of numbering for reference Protein Data Bank
(PDB; rcsb.org) TCRCα 6U07_A and Kabat and International
Immunogenetics Information System (IMGT; imgt.org) T-
cell receptor numbering schemes. Related to FIG. 14A.

| | | | | Residue | Residue |
|---|---|---|---|---|---|
| IMGT DOMAIN | IMGT | Kabat | PDB | [1 letter] | [3 letter] |
| V-ALPHA [D1] | 128V | — | 118 | P | PRO |
| C-ALPHA [D1] | 1.5C | 117 | 119 | Y | TYR |
| C-ALPHA [D1] | 1.4C | 118 | 120 | I | ILE |
| C-ALPHA [D1] | 1.3C | 119 | 121 | Q | GLN |
| C-ALPHA [D1] | 1.2C | 120 | 122 | N | ASN |
| C-ALPHA [D1] | 1.1C | 121 | 123 | P | PRO |
| C-ALPHA [D1] | 1C | 122 | 124 | D | ASP |
| C-ALPHA [D1] | 2C | 123 | 125 | P | PRO |
| C-ALPHA [D1] | 3C | 124 | 126 | A | ALA |
| C-ALPHA [D1] | 4C | 125 | 127 | V | VAL |
| C-ALPHA [D1] | 5C | 126 | 128 | Y | TYR |
| C-ALPHA [D1] | 6C | 127 | 129 | Q | GLN |
| C-ALPHA [D1] | 7C | 128 | 130 | L | LEU |
| C-ALPHA [D1] | 8C | 129 | 131 | R | ARG |
| C-ALPHA [D1] | 9C | 132 | 132 | D | ASP |
| C-ALPHA [D1] | 10C | 133 | 133 | S | SER |
| C-ALPHA [D1] | 11C | 134 | 134 | K | LYS |
| C-ALPHA [D1] | 17C | 135 | 135 | S | SER |
| C-ALPHA [D1] | 18C | 136 | 136 | S | SER |
| C-ALPHA [D1] | 19C | 137 | 137 | D | ASP |
| C-ALPHA [D1] | 20C | 138 | 138 | K | LYS |
| C-ALPHA [D1] | 21C | 139 | 139 | F | PHE |
| C-ALPHA [D1] | 22C | 140 | 140 | V | VAL |
| C-ALPHA [D1] | 23C | 141 | 141 | C | CYS |
| C-ALPHA [D1] | 24C | 142 | 142 | L | LEU |
| C-ALPHA [D1] | 25C | 143 | 143 | F | PHE |
| C-ALPHA [D1] | 26C | 144 | 144 | T | THR |
| C-ALPHA [D1] | 27C | 145 | 145 | D | ASP |
| C-ALPHA [D1] | 28C | 146 | 146 | F | PHE |
| C-ALPHA [D1] | 29C | 147 | 147 | D | ASP |
| C-ALPHA [D1] | 30C | 148 | 148 | S | SER |
| C-ALPHA [D1] | 36C | 149 | 149 | Q | GLN |
| C-ALPHA [D1] | 37C | 150 | 150 | I | ILE |
| C-ALPHA [D1] | 38C | 151 | 151 | N | ASN |
| C-ALPHA [D1] | 39C | 152 | 152 | V | VAL |
| C-ALPHA [D1] | 40C | 153 | 153 | S | SER |
| C-ALPHA [D1] | 41C | 154 | 154 | Q | GLN |
| C-ALPHA [D1] | 42C | 155 | 155 | S | SER |
| C-ALPHA [D1] | 43C | 156 | 156 | K | LYS |
| C-ALPHA [D1] | 44C | 157 | 157 | D | ASP |
| C-ALPHA [D1] | 45C | 158 | 158 | S | SER |
| C-ALPHA [D1] | 77C | 159 | 159 | D | ASP |
| C-ALPHA [D1] | 78C | 160 | 160 | V | VAL |
| C-ALPHA [D1] | 79C | 161 | 161 | Y | TYR |
| C-ALPHA [D1] | 80C | 162 | 162 | I | ILE |
| C-ALPHA [D1] | 81C | 163 | 163 | T | THR |
| C-ALPHA [D1] | 82C | 164 | 164 | D | ASP |
| C-ALPHA [D1] | 83C | 165 | 165 | K | LYS |
| C-ALPHA [D1] | 84C | 166 | 166 | C | CYS |
| C-ALPHA [D1] | 84.1C | 167 | 167 | V | VAL |
| C-ALPHA [D1] | 84.2C | 168 | 168 | L | LEU |
| C-ALPHA [D1] | 84.3C | 169 | 169 | D | ASP |
| C-ALPHA [D1] | 84.4C | 170 | 170 | M | MET |
| C-ALPHA [D1] | 84.5C | 171 | 171 | R | ARG |
| C-ALPHA [D1] | 84.6C | 172 | 172 | S | SER |
| C-ALPHA [D1] | 84.7C | 173 | 173 | M | MET |
| C-ALPHA [D1] | 85.6C | 174 | 174 | D | ASP |
| C-ALPHA [D1] | 85.5C | 175 | 175 | F | PHE |
| C-ALPHA [D1] | 85.4C | 176 | 176 | K | LYS |
| C-ALPHA [D1] | 85.3C | 177 | 177 | S | SER |
| C-ALPHA [D1] | 85.2C | 178 | 178 | N | ASN |
| C-ALPHA [D1] | 85.1C | 179 | 179 | S | SER |
| C-ALPHA [D1] | 85C | 180 | 180 | A | ALA |
| C-ALPHA [D1] | 86C | 181 | 181 | V | VAL |
| C-ALPHA [D1] | 87C | 182 | 182 | A | ALA |
| C-ALPHA [D1] | 88C | 183 | 183 | W | TRP |
| C-ALPHA [D1] | 89C | 184 | 184 | S | SER |
| C-ALPHA [D1] | 90C | 185 | 185 | N | ASN |
| C-ALPHA [D1] | 91C | 186 | 186 | K | LYS |
| C-ALPHA [D1] | 92C | 187 | 187 | S | SER |
| C-ALPHA [D1] | 101C | 188 | 188 | D | ASP |
| C-ALPHA [D1] | 102C | 189 | 189 | F | PHE |
| C-ALPHA [D1] | 103C | 190 | 190 | T | THR |
| C-ALPHA [D1] | 104C | 191 | 191 | C | CYS |
| C-ALPHA [D1] | 105C | 192 | 192 | A | ALA |
| C-ALPHA [D1] | 106C | 193 | 193 | N | ASN |
| C-ALPHA [D1] | 107C | 194 | 194 | A | ALA |
| C-ALPHA [D1] | 108C | 195 | 195 | F | PHE |
| C-ALPHA [D1] | 109C | 196 | 196 | N | ASN |
| C-ALPHA [D1] | 110C | 197 | 197 | N | ASN |
| C-ALPHA [D1] | 113C | 198 | 198 | S | SER |
| C-ALPHA [D1] | 114C | 199 | 199 | I | ILE |
| C-ALPHA [D1] | 115C | 200 | 200 | I | ILE |
| C-ALPHA [D1] | 116C | 201 | 201 | P | PRO |
| C-ALPHA [D1] | 117C | 202 | 202 | E | GLU |
| C-ALPHA [D1] | 118C | 203 | 203 | D | ASP |
| C-ALPHA [D1] | 119C | 204 | 204 | T | THR |
| C-ALPHA [D1] | 120C | 205 | 205 | F | PHE |
| C-ALPHA [D1] | 121C | 206 | 206 | F | PHE |
| C-ALPHA [D1] | 122C | 207 | 207 | P | PRO |
| C-ALPHA [D1] | 123C | 208 | 208 | S | SER |
| C-ALPHA [D1] | 124C | 209 | 209 | P | PRO |
| C-ALPHA [D1] | 125C | 210 | 210 | E | GLU |
| C-ALPHA [D1] | 126C | 211 | 211 | S | SER |
| C-ALPHA [D1] | 127C | 212 | 212 | S | SER |
| C-ALPHA [D1] | 128C | 213 | 213 | C | CYS |

TABLE 7
Alignment of numbering for reference Protein Data Bank
(PDB; rcsb.org) TCRCβ 6U07_B and Kabat and International
Immunogenetics Information System (IMGT; imgt.org) T-
cell receptor numbering schemes. Related to FIG. 14B.

| | | | | Residue | Residue |
|---|---|---|---|---|---|
| IMGT DOMAIN | IMGT | Kabat | PDB | [1 letter] | [3 letter] |
| V-ALPHA [D1] | 128V | — | 118 | P | PRO |
| C-ALPHA [D1] | 1.5C | 117 | 119 | Y | TYR |
| C-ALPHA [D1] | 1.4C | 118 | 120 | I | ILE |
| C-ALPHA [D1] | 1.3C | 119 | 121 | Q | GLN |
| C-ALPHA [D1] | 1.2C | 120 | 122 | N | ASN |
| C-ALPHA [D1] | 1.1C | 121 | 123 | P | PRO |
| C-ALPHA [D1] | 1C | 122 | 124 | D | ASP |
| C-ALPHA [D1] | 2C | 123 | 125 | P | PRO |
| C-ALPHA [D1] | 3C | 124 | 126 | A | ALA |
| C-ALPHA [D1] | 4C | 125 | 127 | V | VAL |
| C-ALPHA [D1] | 5C | 126 | 128 | Y | TYR |
| C-ALPHA [D1] | 6C | 127 | 129 | Q | GLN |
| C-ALPHA [D1] | 7C | 128 | 130 | L | LEU |
| C-ALPHA [D1] | 8C | 129 | 131 | R | ARG |
| C-ALPHA [D1] | 9C | 132 | 132 | D | ASP |
| C-ALPHA [D1] | 10C | 133 | 133 | S | SER |
| C-ALPHA [D1] | 11C | 134 | 134 | K | LYS |
| C-ALPHA [D1] | 17C | 135 | 135 | S | SER |
| C-ALPHA [D1] | 18C | 136 | 136 | S | SER |
| C-ALPHA [D1] | 19C | 137 | 137 | D | ASP |
| C-ALPHA [D1] | 20C | 138 | 138 | K | LYS |
| C-ALPHA [D1] | 21C | 139 | 139 | F | PHE |
| C-ALPHA [D1] | 22C | 140 | 140 | V | VAL |
| C-ALPHA [D1] | 23C | 141 | 141 | C | CYS |
| C-ALPHA [D1] | 24C | 142 | 142 | L | LEU |
| C-ALPHA [D1] | 25C | 143 | 143 | F | PHE |
| C-ALPHA [D1] | 26C | 144 | 144 | T | THR |
| C-ALPHA [D1] | 27C | 145 | 145 | D | ASP |
| C-ALPHA [D1] | 28C | 146 | 146 | F | PHE |
| C-ALPHA [D1] | 29C | 147 | 147 | D | ASP |
| C-ALPHA [D1] | 30C | 148 | 148 | S | SER |
| C-ALPHA [D1] | 36C | 149 | 149 | Q | GLN |
| C-ALPHA [D1] | 37C | 150 | 150 | I | ILE |
| C-ALPHA [D1] | 38C | 151 | 151 | N | ASN |
| C-ALPHA [D1] | 39C | 152 | 152 | V | VAL |
| C-ALPHA [D1] | 40C | 153 | 153 | S | SER |
| C-ALPHA [D1] | 41C | 154 | 154 | Q | GLN |
| C-ALPHA [D1] | 42C | 155 | 155 | S | SER |
| C-ALPHA [D1] | 43C | 156 | 156 | K | LYS |
| C-ALPHA [D1] | 44C | 157 | 157 | D | ASP |
| C-ALPHA [D1] | 45C | 158 | 158 | S | SER |
| C-ALPHA [D1] | 77C | 159 | 159 | D | ASP |
| C-ALPHA [D1] | 78C | 160 | 160 | V | VAL |
| C-ALPHA [D1] | 79C | 161 | 161 | Y | TYR |
| C-ALPHA [D1] | 80C | 162 | 162 | I | ILE |
| C-ALPHA [D1] | 81C | 163 | 163 | T | THR |
| C-ALPHA [D1] | 82C | 164 | 164 | D | ASP |
| C-ALPHA [D1] | 83C | 165 | 165 | K | LYS |
| C-ALPHA [D1] | 84C | 166 | 166 | C | CYS |
| C-ALPHA [D1] | 84.1C | 167 | 167 | V | VAL |
| C-ALPHA [D1] | 84.2C | 168 | 168 | L | LEU |
| C-ALPHA [D1] | 84.3C | 169 | 169 | D | ASP |
| C-ALPHA [D1] | 84.4C | 170 | 170 | M | MET |
| C-ALPHA [D1] | 84.5C | 171 | 171 | R | ARG |
| C-ALPHA [D1] | 84.6C | 172 | 172 | S | SER |
| C-ALPHA [D1] | 84.7C | 173 | 173 | M | MET |
| C-ALPHA [D1] | 85.6C | 174 | 174 | D | ASP |
| C-ALPHA [D1] | 85.5C | 175 | 175 | F | PHE |
| C-ALPHA [D1] | 85.4C | 176 | 176 | K | LYS |
| C-ALPHA [D1] | 85.3C | 177 | 177 | S | SER |
| C-ALPHA [D1] | 85.2C | 178 | 178 | N | ASN |
| C-ALPHA [D1] | 85.1C | 179 | 179 | S | SER |
| C-ALPHA [D1] | 85C | 180 | 180 | A | ALA |
| C-ALPHA [D1] | 86C | 181 | 181 | V | VAL |
| C-ALPHA [D1] | 87C | 182 | 182 | A | ALA |
| C-ALPHA [D1] | 88C | 183 | 183 | W | TRP |
| C-ALPHA [D1] | 89C | 184 | 184 | S | SER |
| C-ALPHA [D1] | 90C | 185 | 185 | N | ASN |
| C-ALPHA [D1] | 91C | 186 | 186 | K | LYS |
| C-ALPHA [D1] | 92C | 187 | 187 | S | SER |
| C-ALPHA [D1] | 101C | 188 | 188 | D | ASP |
| C-ALPHA [D1] | 102C | 189 | 189 | F | PHE |
| C-ALPHA [D1] | 103C | 190 | 190 | T | THR |
| C-ALPHA [D1] | 104C | 191 | 191 | C | CYS |
| C-ALPHA [D1] | 105C | 192 | 192 | A | ALA |
| C-ALPHA [D1] | 106C | 193 | 193 | N | ASN |
| C-ALPHA [D1] | 107C | 194 | 194 | A | ALA |
| C-ALPHA [D1] | 108C | 195 | 195 | F | PHE |
| C-ALPHA [D1] | 109C | 196 | 196 | N | ASN |
| C-ALPHA [D1] | 110C | 197 | 197 | N | ASN |
| C-ALPHA [D1] | 113C | 198 | 198 | S | SER |
| C-ALPHA [D1] | 114C | 199 | 199 | I | ILE |
| C-ALPHA [D1] | 115C | 200 | 200 | I | ILE |
| C-ALPHA [D1] | 116C | 201 | 201 | P | PRO |
| C-ALPHA [D1] | 117C | 202 | 202 | E | GLU |
| C-ALPHA [D1] | 118C | 203 | 203 | D | ASP |
| C-ALPHA [D1] | 119C | 204 | 204 | T | THR |
| C-ALPHA [D1] | 120C | 205 | 205 | F | PHE |
| C-ALPHA [D1] | 121C | 206 | 206 | F | PHE |
| C-ALPHA [D1] | 122C | 207 | 207 | P | PRO |
| C-ALPHA [D1] | 123C | 208 | 208 | S | SER |
| C-ALPHA [D1] | 124C | 209 | 209 | P | PRO |
| C-ALPHA [D1] | 125C | 210 | 210 | E | GLU |
| C-ALPHA [D1] | 126C | 211 | 211 | S | SER |
| C-ALPHA [D1] | 127C | 212 | 212 | S | SER |
| C-ALPHA [D1] | 128C | 213 | 213 | C | CYS |

TABLE 8
Alignment of numbering for reference Protein Data Bank (PDB; rcsb.org) TCRVα
2F53_D and Kabat and International Immunogenetics Information System (IMGT;
imgt.org) T-cell receptor numbering schemes. Related to FIG. 14C.

| | | | | | Residue | Residue |
|---|---|---|---|---|---|---|
| IMGT DOMAIN | FR/CDR | IMGT | Kabat | PDB | [1 letter] | [3 letter] |
| — | — | — | — | −1 | M | MET |
| V-ALPHA [D1] | FR1 | 1V | 1 | 0 | K | LYS |
| V-ALPHA [D1] | FR1 | 2V | 2 | 1 | Q | GLN |
| V-ALPHA [D1] | FR1 | 3V | 3 | 2 | E | GLU |
| V-ALPHA [D1] | FR1 | 4V | 4 | 3 | V | VAL |
| V-ALPHA [D1] | FR1 | 5V | 5 | 4 | T | THR |
| V-ALPHA [D1] | FR1 | 6V | 6 | 5 | Q | GLN |
| V-ALPHA [D1] | FR1 | 7V | 7 | 6 | I | ILE |
| V-ALPHA [D1] | FR1 | 8V | 8 | 7 | P | PRO |
| V-ALPHA [D1] | FR1 | 9V | 9 | 8 | A | ALA |
| V-ALPHA [D1] | FR1 | 10V | 10 | 9 | A | ALA |
| V-ALPHA [D1] | FR1 | 11V | 11 | 10 | L | LEU |
| V-ALPHA [D1] | FR1 | 12V | 12 | 11 | S | SER |
| V-ALPHA [D1] | FR1 | 13V | 13 | 12 | V | VAL |
| V-ALPHA [D1] | FR1 | 14V | 14 | 13 | P | PRO |
| V-ALPHA [D1] | FR1 | 15V | 15 | 14 | E | GLU |
| V-ALPHA [D1] | FR1 | 16V | 16 | 15 | G | GLY |
| V-ALPHA [D1] | FR1 | 17V | 17 | 16 | E | GLU |
| V-ALPHA [D1] | FR1 | 18V | 18 | 17 | N | ASN |
| V-ALPHA [D1] | FR1 | 19V | 19 | 18 | L | LEU |
| V-ALPHA [D1] | FR1 | 20V | 20 | 19 | V | VAL |
| V-ALPHA [D1] | FR1 | 21V | 21 | 20 | L | LEU |
| V-ALPHA [D1] | FR1 | 22V | 22 | 21 | N | ASN |
| V-ALPHA [D1] | FR1 | 23V | 23 | 22 | C | CYS |
| V-ALPHA [D1] | FR1 | 24V | 24 | 23 | S | SER |
| V-ALPHA [D1] | FR1 | 25V | 25 | 24 | F | PHE |
| V-ALPHA [D1] | FR1 | 26V | 26 | 25 | T | THR |
| V-ALPHA [D1] | CDR1 | 27V | 27 | 26 | D | ASP |
| V-ALPHA [D1] | CDR1 | 28V | 29 | 27 | S | SER |
| V-ALPHA [D1] | CDR1 | 29V | 30 | 28 | A | ALA |
| — | — | 30 | — | — | — | — |
| — | — | 31 | — | — | — | — |
| — | — | 32 | — | — | — | — |
| — | — | 33 | — | — | — | — |
| — | — | 34 | — | — | — | — |
| — | — | 35 | — | — | — | — |
| V-ALPHA [D1] | CDR1 | 36V | 30a | 29 | I | ILE |
| V-ALPHA [D1] | CDR1 | 37V | 30b | 30 | Y | TYR |
| V-ALPHA [D1] | CDR1 | 38V | 31 | 31 | N | ASN |
| V-ALPHA [D1] | FR2 | 39V | 32 | 32 | L | LEU |
| V-ALPHA [D1] | FR2 | 40V | 33 | 33 | Q | GLN |
| V-ALPHA [D1] | FR2 | 41V | 34 | 34 | W | TRP |
| V-ALPHA [D1] | FR2 | 42V | 35 | 35 | F | PHE |
| V-ALPHA [D1] | FR2 | 43V | 36 | 36 | R | ARG |
| V-ALPHA [D1] | FR2 | 44V | 37 | 37 | Q | GLN |
| V-ALPHA [D1] | FR2 | 45V | 38 | 38 | D | ASP |
| V-ALPHA [D1] | FR2 | 46V | 39 | 39 | P | PRO |
| V-ALPHA [D1] | FR2 | 47V | 40 | 40 | G | GLY |
| V-ALPHA [D1] | FR2 | 48V | 41 | 41 | K | LYS |
| V-ALPHA [D1] | FR2 | 49V | 42 | 42 | G | GLY |
| V-ALPHA [D1] | FR2 | 50V | 43 | 43 | L | LEU |
| V-ALPHA [D1] | FR2 | 51V | 44 | 44 | T | THR |
| V-ALPHA [D1] | FR2 | 52V | 45 | 45 | S | SER |
| V-ALPHA [D1] | FR2 | 53V | 46 | 46 | L | LEU |
| V-ALPHA [D1] | FR2 | 54V | 47 | 47 | L | LEU |
| V-ALPHA [D1] | FR2 | 55V | 48 | 48 | L | LEU |
| V-ALPHA [D1] | CDR2 | 56V | 49 | 49 | I | ILE |
| V-ALPHA [D1] | CDR2 | 57V | 50 | 50 | P | PRO |
| V-ALPHA [D1] | CDR2 | 58V | — | 51 | F | PHE |
| V-ALPHA [D1] | CDR2 | 59V | — | 52 | W | TRP |
| — | — | 60 | — | — | — | 0 |
| — | — | 61 | — | — | — | — |
| — | — | 62 | — | — | — | — |
| V-ALPHA [D1] | CDR2 | 63V | 55 | 53 | Q | GLN |
| V-ALPHA [D1] | CDR2 | 64V | 56 | 54 | R | ARG |
| V-ALPHA [D1] | CDR2 | 65V | 57 | 55 | E | GLU |
| V-ALPHA [D1] | FR3 | 66V | 58 | 56 | Q | GLN |
| V-ALPHA [D1] | FR3 | 67V | 59 | 57 | T | THR |
| V-ALPHA [D1] | FR3 | 68V | — | 58 | S | SER |
| — | — | 69 | — | — | — | — |
| — | — | 70 | — | — | — | — |
| — | — | 71 | — | — | — | — |
| — | — | 72 | — | — | — | — |
| — | — | 73 | — | — | — | — |
| V-ALPHA [D1] | FR3 | 74V | 60 | 59 | G | GLY |
| V-ALPHA [D1] | FR3 | 75V | 61 | 60 | R | ARG |
| V-ALPHA [D1] | FR3 | 76V | 62 | 61 | L | LEU |
| V-ALPHA [D1] | FR3 | 77V | 63 | 62 | N | ASN |
| V-ALPHA [D1] | FR3 | 78V | 64 | 63 | A | ALA |
| V-ALPHA [D1] | FR3 | 79V | 65 | 64 | S | SER |
| V-ALPHA [D1] | FR3 | 80V | 66 | 65 | L | LEU |
| V-ALPHA [D1] | FR3 | 81V | 67 | 66 | D | ASP |
| V-ALPHA [D1] | FR3 | 82V | 68 | 67 | K | LYS |
| V-ALPHA [D1] | FR3 | 83V | 69 | 68 | S | SER |
| V-ALPHA [D1] | FR3 | 84V | 70 | 69 | S | SER |
| V-ALPHA [D1] | FR3 | 85V | 71 | 70 | G | GLY |
| V-ALPHA [D1] | FR3 | 86V | 72 | 71 | R | ARG |
| V-ALPHA [D1] | FR3 | 87V | 73 | 72 | S | SER |
| V-ALPHA [D1] | FR3 | 88V | 74 | 73 | T | THR |
| V-ALPHA [D1] | FR3 | 89V | 75 | 74 | L | LEU |
| V-ALPHA [D1] | FR3 | 90V | 76 | 75 | Y | TYR |
| V-ALPHA [D1] | FR3 | 91V | 77 | 76 | I | ILE |
| V-ALPHA [D1] | FR3 | 92V | 78 | 77 | A | ALA |
| V-ALPHA [D1] | FR3 | 93V | 79 | 78 | A | ALA |
| V-ALPHA [D1] | FR3 | 94V | 80 | 79 | S | SER |
| V-ALPHA [D1] | FR3 | 95V | 81 | 80 | Q | GLN |
| V-ALPHA [D1] | FR3 | 96V | 82 | 81 | P | PRO |
| V-ALPHA [D1] | FR3 | 97V | 83 | 82 | G | GLY |
| V-ALPHA [D1] | FR3 | 98V | 84 | 83 | D | ASP |
| V-ALPHA [D1] | FR3 | 99V | 85 | 84 | S | SER |
| V-ALPHA [D1] | FR3 | 100V | 86 | 85 | A | ALA |
| V-ALPHA [D1] | FR3 | 101V | 87 | 86 | T | THR |
| V-ALPHA [D1] | FR3 | 102V | 88 | 87 | Y | TYR |
| V-ALPHA [D1] | FR3 | 103V | 89 | 88 | L | LEU |
| V-ALPHA [D1] | FR3 | 104V | 90 | 89 | C | CYS |
| V-ALPHA [D1] | CDR3 | 105V | 91 | 90 | A | ALA |
| V-ALPHA [D1] | CDR3 | 106V | 92 | 91 | V | VAL |
| V-ALPHA [D1] | CDR3 | 107V | 93 | 92 | R | ARG |
| V-ALPHA [D1] | CDR3 | 108V | — | 93 | P | PRO |
| V-ALPHA [D1] | CDR3 | 109V | — | 94 | T | THR |
| V-ALPHA [D1] | CDR3 | 110V | — | 95 | S | SER |
| V-ALPHA [D1] | CDR3 | 111V | — | 96 | G | GLY |
| V-ALPHA [D1] | CDR3 | 112V | — | 97 | G | GLY |
| V-ALPHA [D1] | CDR3 | 113V | — | 98 | S | SER |
| V-ALPHA [D1] | CDR3 | 114V | — | 99 | Y | TYR |
| V-ALPHA [D1] | CDR3 | 115V | — | 100 | I | ILE |
| V-ALPHA [D1] | CDR3 | 116V | — | 101 | P | PRO |
| V-ALPHA [D1] | CDR3 | 117V | — | 102 | T | THR |
| V-ALPHA [D1] | FR4 | 118V | — | 103 | F | PHE |
| V-ALPHA [D1] | FR4 | 119V | — | 104 | G | GLY |
| V-ALPHA [D1] | FR4 | 120V | — | 105 | R | ARG |
| V-ALPHA [D1] | FR4 | 121V | — | 106 | G | GLY |
| V-ALPHA [D1] | FR4 | 122V | — | 107 | T | THR |
| V-ALPHA [D1] | FR4 | 123V | — | 108 | S | SER |
| V-ALPHA [D1] | FR4 | 124V | — | 109 | L | LEU |
| V-ALPHA [D1] | FR4 | 125V | — | 110 | I | ILE |
| V-ALPHA [D1] | FR4 | 126V | — | 111 | V | VAL |
| V-ALPHA [D1] | FR4 | 127V | — | 112 | H | HIS |
| V-ALPHA [D1] | FR4 | 128V | — | 113 | P | PRO |
| C-ALPHA [D2] | — | 1.5C | — | 114 | Y | TYR |
| C-ALPHA [D2] | — | 1.4C | — | 115 | I | ILE |
| C-ALPHA [D2] | — | 1.3C | — | 116 | Q | GLN |
| C-ALPHA [D2] | — | 1.2C | — | 117 | N | ASN |
| C-ALPHA [D2] | — | 1.1C | — | 118 | P | PRO |
| C-ALPHA [D2] | — | 1C | — | 119 | D | ASP |
| C-ALPHA [D2] | — | 2C | — | 120 | P | PRO |
| C-ALPHA [D2] | — | 3C | — | 121 | A | ALA |
| C-ALPHA [D2] | — | 4C | — | 122 | V | VAL |
| C-ALPHA [D2] | — | 5C | — | 123 | Y | TYR |
| C-ALPHA [D2] | — | 6C | — | 124 | Q | GLN |
| C-ALPHA [D2] | — | 7C | — | 125 | L | LEU |
| C-ALPHA [D2] | — | 8C | — | 126 | R | ARG |
| C-ALPHA [D2] | — | 9C | — | 127 | D | ASP |
| C-ALPHA [D2] | — | 10C | — | 128 | S | SER |
| C-ALPHA [D2] | — | 11C | — | 129 | K | LYS |
| C-ALPHA [D2] | — | 17C | — | 130 | S | SER |
| C-ALPHA [D2] | — | 18C | — | 131 | S | SER |
| C-ALPHA [D2] | — | 19C | — | 132 | D | ASP |
| C-ALPHA [D2] | — | 20C | — | 133 | K | LYS |
| C-ALPHA [D2] | — | 21C | — | 134 | S | SER |
| C-ALPHA [D2] | — | 22C | — | 135 | V | VAL |
| C-ALPHA [D2] | — | 23C | — | 136 | C | CYS |
| C-ALPHA [D2] | — | 24C | — | 137 | L | LEU |
| C-ALPHA [D2] | — | 25C | — | 138 | F | PHE |
| C-ALPHA [D2] | — | 26C | — | 139 | T | THR |
| C-ALPHA [D2] | — | 27C | — | 140 | D | ASP |
| C-ALPHA [D2] | — | 28C | — | 141 | F | PHE |
| C-ALPHA [D2] | — | 29C | — | 142 | D | ASP |
| C-ALPHA [D2] | — | 30C | — | 143 | S | SER |
| C-ALPHA [D2] | — | 36C | — | 144 | Q | GLN |
| C-ALPHA [D2] | — | 37C | — | 145 | T | THR |
| C-ALPHA [D2] | — | 38C | — | 146 | N | ASN |
| C-ALPHA [D2] | — | 39C | — | 147 | V | VAL |
| C-ALPHA [D2] | — | 40C | — | 148 | S | SER |
| C-ALPHA [D2] | — | 41C | — | 149 | Q | GLN |
| C-ALPHA [D2] | — | 42C | — | 150 | S | SER |
| C-ALPHA [D2] | — | 43C | — | 151 | K | LYS |
| C-ALPHA [D2] | — | 44C | — | 152 | D | ASP |
| C-ALPHA [D2] | — | 45C | — | 153 | S | SER |
| C-ALPHA [D2] | — | 77C | — | 154 | D | ASP |
| C-ALPHA [D2] | — | 78C | — | 155 | V | VAL |
| C-ALPHA [D2] | — | 79C | — | 156 | Y | TYR |
| C-ALPHA [D2] | — | 80C | — | 157 | I | ILE |
| C-ALPHA [D2] | — | 81C | — | 158 | T | THR |
| C-ALPHA [D2] | — | 82C | — | 159 | D | ASP |
| C-ALPHA [D2] | — | 83C | — | 160 | K | LYS |
| C-ALPHA [D2] | — | 84C | — | 161 | C | CYS |
| C-ALPHA [D2] | — | 84.1C | — | 162 | V | VAL |
| C-ALPHA [D2] | — | 84.2C | — | 163 | L | LEU |
| C-ALPHA [D2] | — | 84.3C | — | 164 | D | ASP |
| C-ALPHA [D2] | — | 84.4C | — | 165 | M | MET |
| C-ALPHA [D2] | — | 84.5C | — | 166 | R | ARG |
| C-ALPHA [D2] | — | 84.6C | — | 167 | S | SER |
| C-ALPHA [D2] | — | 84.7C | — | 168 | M | MET |
| C-ALPHA [D2] | — | 85.6C | — | 169 | D | ASP |
| C-ALPHA [D2] | — | 85.5C | — | 170 | F | PHE |
| C-ALPHA [D2] | — | 85.4C | — | 171 | K | LYS |
| C-ALPHA [D2] | — | 85.3C | — | 172 | S | SER |
| C-ALPHA [D2] | — | 85.2C | — | 173 | N | ASN |
| C-ALPHA [D2] | — | 85.1C | — | 174 | S | SER |
| C-ALPHA [D2] | — | 85C | — | 175 | A | ALA |
| C-ALPHA [D2] | — | 86C | — | 176 | V | VAL |
| C-ALPHA [D2] | — | 87C | — | 177 | A | ALA |
| C-ALPHA [D2] | — | 88C | — | 178 | W | TRP |
| C-ALPHA [D2] | — | 89C | — | 179 | S | SER |
| C-ALPHA [D2] | — | 90C | — | 180 | N | ASN |
| C-ALPHA [D2] | — | 91C | — | 181 | K | LYS |
| C-ALPHA [D2] | — | 92C | — | 182 | S | SER |
| C-ALPHA [D2] | — | 101C | — | 183 | D | ASP |
| C-ALPHA [D2] | — | 102C | — | 184 | F | PHE |
| C-ALPHA [D2] | — | 103C | — | 185 | A | ALA |
| C-ALPHA [D2] | — | 104C | — | 186 | C | CYS |
| C-ALPHA [D2] | — | 105C | — | 187 | A | ALA |
| C-ALPHA [D2] | — | 106C | — | 188 | N | ASN |
| C-ALPHA [D2] | — | 107C | — | 189 | A | ALA |
| C-ALPHA [D2] | — | 108C | — | 190 | F | PHE |
| C-ALPHA [D2] | — | 109C | — | 191 | N | ASN |

TABLE 9
Alignment of numbering for reference Protein Data Bank (PDB; rcsb.org) TCRVβ
2F53_E and Kabat and International Immunogenetics Information System (IMGT;
imgt.org) T-cell receptor numbering schemes. Related to FIG. 14D.

| | | | | | Residue | Residue |
|---|---|---|---|---|---|---|
| IMGT DOMAIN | IMGT CDR | IMGT | Kabat | PDB | [1 letter] | [3 letter] |
| V-BETA [D1] | FR1 | 1V | 1 | −1 | N | ASN |
| V-BETA [D1] | FR1 | 2V | 2 | 0 | A | ALA |
| V-BETA [D1] | FR1 | 3V | 3 | 1 | G | GLY |
| V-BETA [D1] | FR1 | 4V | 4 | 2 | V | VAL |
| V-BETA [D1] | FR1 | 5V | 5 | 3 | T | THR |
| V-BETA [D1] | FR1 | 6V | 6 | 4 | Q | GLN |
| V-BETA [D1] | FR1 | 7V | 7 | 5 | T | THR |
| V-BETA [D1] | FR1 | 8V | 8 | 6 | P | PRO |
| V-BETA [D1] | FR1 | 9V | 9 | 7 | K | LYS |
| V-BETA [D1] | FR1 | 10V | 10 | 8 | F | PHE |
| V-BETA [D1] | FR1 | 11V | 11 | 9 | Q | GLN |
| V-BETA [D1] | FR1 | 12V | 12 | 10 | V | VAL |
| V-BETA [D1] | FR1 | 13V | 13 | 11 | L | LEU |
| V-BETA [D1] | FR1 | 14V | 14 | 12 | K | LYS |
| V-BETA [D1] | FR1 | 15V | 15 | 13 | T | THR |
| V-BETA [D1] | FR1 | 16V | 16 | 14 | G | GLY |
| V-BETA [D1] | FR1 | 17V | 17 | 15 | Q | GLN |
| V-BETA [D1] | FR1 | 18V | 18 | 16 | S | SER |
| V-BETA [D1] | FR1 | 19V | 19 | 17 | M | MET |
| V-BETA [D1] | FR1 | 20V | 20 | 18 | T | THR |
| V-BETA [D1] | FR1 | 21V | 21 | 19 | L | LEU |
| V-BETA [D1] | FR1 | 22V | 22 | 20 | Q | GLN |
| V-BETA [D1] | FR1 | 23V | 23 | 21 | C | CYS |
| V-BETA [D1] | FR1 | 24V | 24 | 22 | A | ALA |
| V-BETA [D1] | FR1 | 25V | 25 | 23 | Q | GLN |
| V-BETA [D1] | FR1 | 26V | 26 | 24 | D | ASP |
| V-BETA [D1] | CDR1 | 27V | 27 | 25 | M | MET |
| V-BETA [D1] | CDR1 | 28V | 28 | 26 | N | ASN |
| V-BETA [D1] | CDR1 | 29V | 29 | 27 | H | HIS |
| — | CDR1 | 30 | — | — | — | — |
| — | CDR1 | 31 | — | — | — | — |
| — | CDR1 | 32 | — | — | — | — |
| — | CDR1 | 33 | — | — | — | — |
| — | CDR1 | 34 | — | — | — | — |
| — | CDR1 | 35 | — | — | — | — |
| — | CDR1 | 36 | — | — | — | — |
| V-BETA [D1] | CDR1 | 37V | 30 | 28 | E | GLU |
| V-BETA [D1] | CDR1 | 38V | 31 | 29 | Y | TYR |
| V-BETA [D1] | FR2 | 39V | 32 | 30 | M | MET |
| V-BETA [D1] | FR2 | 40V | 33 | 31 | S | SER |
| V-BETA [D1] | FR2 | 41V | 34 | 32 | W | TRP |
| V-BETA [D1] | FR2 | 42V | 35 | 33 | Y | TYR |
| V-BETA [D1] | FR2 | 43V | 36 | 34 | R | ARG |
| V-BETA [D1] | FR2 | 44V | 37 | 35 | Q | GLN |
| V-BETA [D1] | FR2 | 45V | 38 | 36 | D | ASP |
| V-BETA [D1] | FR2 | 46V | 39 | 37 | P | PRO |
| V-BETA [D1] | FR2 | 47V | 40 | 38 | G | GLY |
| V-BETA [D1] | FR2 | 48V | 41 | 39 | M | MET |
| V-BETA [D1] | FR2 | 49V | 42 | 40 | G | GLY |
| V-BETA [D1] | FR2 | 50V | 43 | 41 | L | LEU |
| V-BETA [D1] | FR2 | 51V | 44 | 42 | R | ARG |
| V-BETA [D1] | FR2 | 52V | 45 | 43 | L | LEU |
| V-BETA [D1] | FR2 | 53V | 46 | 44 | I | ILE |
| V-BETA [D1] | FR2 | 54V | 47 | 45 | H | HIS |
| V-BETA [D1] | FR2 | 55V | 48 | 46 | Y | TYR |
| V-BETA [D1] | CDR2 | 56V | 49 | 47 | S | SER |
| V-BETA [D1] | CDR2 | 57V | 50 | 48 | V | VAL |
| V-BETA [D1] | CDR2 | 58V | 51 | 49 | S | SER |
| — | CDR2 | 59 | — | — | — | — |
| — | CDR2 | 60 | — | — | — | — |
| — | CDR2 | 61 | — | — | — | — |
| — | CDR2 | 62 | — | — | — | — |
| V-BETA [D1] | CDR2 | 63V | 52 | 50 | V | VAL |
| V-BETA [D1] | CDR2 | 64V | 53 | 51 | G | GLY |
| V-BETA [D1] | CDR2 | 65V | 54 | 52 | M | MET |
| V-BETA [D1] | FR3 | 66V | 55 | 53 | T | THR |
| V-BETA [D1] | FR3 | 67V | 56 | 54 | D | ASP |
| V-BETA [D1] | FR3 | 68V | 57 | 55 | Q | GLN |
| V-BETA [D1] | FR3 | 69V | 58 | 56 | G | GLY |
| V-BETA [D1] | FR3 | 70V | 59 | 57 | E | GLU |
| V-BETA [D1] | FR3 | 71V | 60 | 58 | V | VAL |
| V-BETA [D1] | FR3 | 72V | 61 | 59 | P | PRO |
| — | FR3 | 73 | 62 | — | — | — |
| V-BETA [D1] | FR3 | 74V | 63 | 60 | N | ASN |
| V-BETA [D1] | FR3 | 75V | 64 | 61 | G | GLY |
| V-BETA [D1] | FR3 | 76V | 65 | 62 | Y | TYR |
| V-BETA [D1] | FR3 | 77V | 66 | 63 | N | ASN |
| V-BETA [D1] | FR3 | 78V | 67 | 64 | V | VAL |
| V-BETA [D1] | FR3 | 79V | 68 | 65 | S | SER |
| V-BETA [D1] | FR3 | 80V | 69 | 66 | R | ARG |
| V-BETA [D1] | FR3 | 81V | 70 | 67 | S | SER |
| — | FR3 | 82 | — | — | — | — |
| V-BETA [D1] | FR3 | 83V | 71 | 68 | T | THR |
| V-BETA [D1] | FR3 | 84V | 72 | 69 | T | THR |
| V-BETA [D1] | FR3 | 85V | 73 | 70 | E | GLU |
| V-BETA [D1] | FR3 | 86V | 74 | 71 | D | ASP |
| V-BETA [D1] | FR3 | 87V | 75 | 72 | F | PHE |
| V-BETA [D1] | FR3 | 88V | 76 | 73 | P | PRO |
| V-BETA [D1] | FR3 | 89V | 77 | 74 | L | LEU |
| V-BETA [D1] | FR3 | 90V | 78 | 75 | R | ARG |
| V-BETA [D1] | FR3 | 91V | 79 | 76 | L | LEU |
| V-BETA [D1] | FR3 | 92V | 80 | 77 | L | LEU |
| V-BETA [D1] | FR3 | 93V | 81 | 78 | S | SER |
| V-BETA [D1] | FR3 | 94V | 82 | 79 | A | ALA |
| V-BETA [D1] | FR3 | 95V | 83 | 80 | A | ALA |
| V-BETA [D1] | FR3 | 96V | 84 | 81 | P | PRO |
| V-BETA [D1] | FR3 | 97V | 85 | 82 | S | SER |
| V-BETA [D1] | FR3 | 98V | 86 | 83 | Q | GLN |
| V-BETA [D1] | FR3 | 99V | 87 | 84 | T | THR |
| V-BETA [D1] | FR3 | 100V | 88 | 85 | S | SER |
| V-BETA [D1] | FR3 | 101V | 89 | 86 | V | VAL |
| V-BETA [D1] | FR3 | 102V | 90 | 87 | Y | TYR |
| V-BETA [D1] | FR3 | 103V | 91 | 88 | F | PHE |
| V-BETA [D1] | FR3 | 104V | 92 | 89 | C | CYS |
| V-BETA [D1] | CDR3 | 105V | 93 | 90 | A | ALA |
| V-BETA [D1] | CDR3 | 106V | 94 | 91 | S | SER |
| V-BETA [D1] | CDR3 | 107V | 95 | 92 | S | SER |
| V-BETA [D1] | CDR3 | 108V | 96 | 93 | Y | TYR |
| V-BETA [D1] | CDR3 | 109V | — | 94 | V | VAL |
| V-BETA [D1] | CDR3 | 110V | — | 95 | G | GLY |
| — | CDR3 | 111 | — | — | — | — |
| V-BETA [D1] | CDR3 | 112V | — | 96 | N | ASN |
| V-BETA [D1] | CDR3 | 113V | — | 97 | T | THR |
| V-BETA [D1] | CDR3 | 114V | — | 98 | G | GLY |
| V-BETA [D1] | CDR3 | 115V | — | 99 | E | GLU |
| V-BETA [D1] | CDR3 | 116V | — | 100 | L | LEU |
| V-BETA [D1] | CDR3 | 117V | — | 101 | F | PHE |
| V-BETA [D1] | FR4 | 118V | — | 102 | F | PHE |
| V-BETA [D1] | FR4 | 119V | — | 103 | G | GLY |
| V-BETA [D1] | FR4 | 120V | — | 104 | E | GLU |
| V-BETA [D1] | FR4 | 121V | — | 105 | G | GLY |
| V-BETA [D1] | FR4 | 122V | — | 106 | S | SER |
| V-BETA [D1] | FR4 | 123V | — | 107 | R | ARG |
| V-BETA [D1] | FR4 | 124V | — | 108 | L | LEU |
| V-BETA [D1] | FR4 | 125V | — | 109 | T | THR |
| V-BETA [D1] | FR4 | 126V | — | 110 | V | VAL |
| V-BETA [D1] | FR4 | 127V | — | 111 | L | LEU |
| C-BETA-2 [D2] | — | 1.7C | — | 112 | E | GLU |
| C-BETA-2 [D2] | — | 1.6C | — | 113 | D | ASP |
| C-BETA-2 [D2] | — | 1.5C | — | 114 | L | LEU |
| C-BETA-2 [D2] | — | 1.4C | — | 115 | K | LYS |
| C-BETA-2 [D2] | — | 1.3C | — | 116 | N | ASN |
| C-BETA-2 [D2] | — | 1.2C | — | 117 | V | VAL |
| C-BETA-2 [D2] | — | 1.1C | — | 118 | F | PHE |
| C-BETA-2 [D2] | — | 1C | — | 119 | P | PRO |
| C-BETA-2 [D2] | — | 2C | — | 120 | P | PRO |
| C-BETA-2 [D2] | — | 3C | — | 121 | E | GLU |
| C-BETA-2 [D2] | — | 4C | — | 122 | V | VAL |
| C-BETA-2 [D2] | — | 5C | — | 123 | A | ALA |
| C-BETA-2 [D2] | — | 6C | — | 124 | V | VAL |
| C-BETA-2 [D2] | — | 7C | — | 125 | F | PHE |
| C-BETA-2 [D2] | — | 8C | — | 126 | E | GLU |
| C-BETA-2 [D2] | — | 9C | — | 127 | P | PRO |
| C-BETA-2 [D2] | — | 10C | — | 128 | S | SER |
| C-BETA-2 [D2] | — | 11C | — | 129 | E | GLU |
| C-BETA-2 [D2] | — | 12C | — | 130 | A | ALA |
| C-BETA-2 [D2] | — | 13C | — | 131 | E | GLU |
| C-BETA-2 [D2] | — | 14C | — | 132 | I | ILE |
| C-BETA-2 [D2] | — | 15C | — | 133 | S | SER |
| C-BETA-2 [D2] | — | 15.1C | — | 134 | H | HIS |
| C-BETA-2 [D2] | — | 16C | — | 135 | T | THR |
| C-BETA-2 [D2] | — | 17C | — | 136 | Q | GLN |
| C-BETA-2 [D2] | — | 18C | — | 137 | K | LYS |
| C-BETA-2 [D2] | — | 19C | — | 138 | A | ALA |
| C-BETA-2 [D2] | — | 20C | — | 139 | T | THR |
| C-BETA-2 [D2] | — | 21C | — | 140 | L | LEU |
| C-BETA-2 [D2] | — | 22C | — | 141 | V | VAL |
| C-BETA-2 [D2] | — | 23C | — | 142 | C | CYS |
| C-BETA-2 [D2] | — | 24C | — | 143 | L | LEU |
| C-BETA-2 [D2] | — | 25C | — | 144 | A | ALA |
| C-BETA-2 [D2] | — | 26C | — | 145 | T | THR |
| C-BETA-2 [D2] | — | 27C | — | 146 | G | GLY |
| C-BETA-2 [D2] | — | 28C | — | 147 | F | PHE |
| C-BETA-2 [D2] | — | 29C | — | 148 | Y | TYR |
| C-BETA-2 [D2] | — | 30C | — | 149 | P | PRO |
| C-BETA-2 [D2] | — | 35C | — | 150 | D | ASP |
| C-BETA-2 [D2] | — | 36C | — | 151 | H | HIS |
| C-BETA-2 [D2] | — | 37C | — | 152 | V | VAL |
| C-BETA-2 [D2] | — | 38C | — | 153 | E | GLU |
| C-BETA-2 [D2] | — | 39C | — | 154 | L | LEU |
| C-BETA-2 [D2] | — | 40C | — | 155 | S | SER |
| C-BETA-2 [D2] | — | 41C | — | 156 | W | TRP |
| C-BETA-2 [D2] | — | 42C | — | 157 | W | TRP |
| C-BETA-2 [D2] | — | 43C | — | 158 | V | VAL |
| C-BETA-2 [D2] | — | 44C | — | 159 | N | ASN |
| C-BETA-2 [D2] | — | 45C | — | 160 | G | GLY |
| C-BETA-2 [D2] | — | 45.1C | — | 161 | K | LYS |
| C-BETA-2 [D2] | — | 45.2C | — | 162 | E | GLU |
| C-BETA-2 [D2] | — | 45.3C | — | 163 | V | VAL |
| C-BETA-2 [D2] | — | 45.4C | — | 164 | H | HIS |
| C-BETA-2 [D2] | — | 45.5C | — | 165 | S | SER |
| C-BETA-2 [D2] | — | 77C | — | 166 | G | GLY |
| C-BETA-2 [D2] | — | 78C | — | 167 | V | VAL |
| C-BETA-2 [D2] | — | 79C | — | 168 | C | CYS |
| C-BETA-2 [D2] | — | 80C | — | 169 | T | THR |
| C-BETA-2 [D2] | — | 81C | — | 170 | D | ASP |
| C-BETA-2 [D2] | — | 82C | — | 171 | P | PRO |
| C-BETA-2 [D2] | — | 83C | — | 172 | Q | GLN |
| C-BETA-2 [D2] | — | 84C | — | 173 | P | PRO |
| C-BETA-2 [D2] | — | 84.1C | — | 174 | L | LEU |
| C-BETA-2 [D2] | — | 84.2C | — | 175 | K | LYS |
| C-BETA-2 [D2] | — | 84.3C | — | 176 | E | GLU |
| C-BETA-2 [D2] | — | 84.4C | — | 177 | Q | GLN |
| C-BETA-2 [D2] | — | 84.5C | — | 178 | P | PRO |
| C-BETA-2 [D2] | — | 84.6C | — | 179 | A | ALA |
| C-BETA-2 [D2] | — | 84.7C | — | 180 | L | LEU |
| C-BETA-2 [D2] | — | 85.6C | — | 181 | N | ASN |
| C-BETA-2 [D2] | — | 85.5C | — | 182 | D | ASP |
| C-BETA-2 [D2] | — | 85.4C | — | 183 | S | SER |
| C-BETA-2 [D2] | — | 85.3C | — | 184 | R | ARG |
| C-BETA-2 [D2] | — | 85.2C | — | 185 | Y | TYR |
| C-BETA-2 [D2] | — | 85.1C | — | 186 | A | ALA |
| C-BETA-2 [D2] | — | 85C | — | 187 | L | LEU |
| C-BETA-2 [D2] | — | 86C | — | 188 | S | SER |
| C-BETA-2 [D2] | — | 87C | — | 189 | S | SER |
| C-BETA-2 [D2] | — | 88C | — | 190 | R | ARG |
| C-BETA-2 [D2] | — | 89C | — | 191 | L | LEU |
| C-BETA-2 [D2] | — | 90C | — | 192 | R | ARG |
| C-BETA-2 [D2] | — | 91C | — | 193 | V | VAL |
| C-BETA-2 [D2] | — | 92C | — | 194 | S | SER |
| C-BETA-2 [D2] | — | 93C | — | 195 | A | ALA |
| C-BETA-2 [D2] | — | 94C | — | 196 | T | THR |
| C-BETA-2 [D2] | — | 95C | — | 197 | F | PHE |
| C-BETA-2 [D2] | — | 96C | — | 198 | W | TRP |
| C-BETA-2 [D2] | — | 96.1C | — | 199 | Q | GLN |
| C-BETA-2 [D2] | — | 97C | — | 200 | D | ASP |
| C-BETA-2 [D2] | — | 98C | — | 201 | P | PRO |
| C-BETA-2 [D2] | — | 99C | — | 202 | R | ARG |
| C-BETA-2 [D2] | — | 100C | — | 203 | N | ASN |
| C-BETA-2 [D2] | — | 101C | — | 204 | H | HIS |
| C-BETA-2 [D2] | — | 102C | — | 205 | F | PHE |
| C-BETA-2 [D2] | — | 103C | — | 206 | R | ARG |
| C-BETA-2 [D2] | — | 104C | — | 207 | C | CYS |
| C-BETA-2 [D2] | — | 105C | — | 208 | Q | GLN |
| C-BETA-2 [D2] | — | 106C | — | 209 | V | VAL |
| C-BETA-2 [D2] | — | 107C | — | 210 | Q | GLN |
| C-BETA-2 [D2] | — | 108C | — | 211 | F | PHE |
| C-BETA-2 [D2] | — | 109C | — | 212 | Y | TYR |
| C-BETA-2 [D2] | — | 110C | — | 213 | G | GLY |
| C-BETA-2 [D2] | — | 111C | — | 214 | L | LEU |
| C-BETA-2 [D2] | — | 111.1C | — | 215 | S | SER |
| C-BETA-2 [D2] | — | 111.2C | — | 216 | E | GLU |
| C-BETA-2 [D2] | — | 111.3C | — | 217 | N | ASN |
| C-BETA-2 [D2] | — | 111.4C | — | 218 | D | ASP |
| C-BETA-2 [D2] | — | 111.5C | — | 219 | E | GLU |
| C-BETA-2 [D2] | — | 111.6C | — | 220 | W | TRP |
| C-BETA-2 [D2] | — | 112.6C | — | 221 | T | THR |
| C-BETA-2 [D2] | — | 112.5C | — | 222 | Q | GLN |
| C-BETA-2 [D2] | — | 112.4C | — | 223 | D | ASP |
| C-BETA-2 [D2] | — | 112.3C | — | 224 | R | ARG |
| C-BETA-2 [D2] | — | 112.2C | — | 225 | A | ALA |
| C-BETA-2 [D2] | — | 112.1C | — | 226 | K | LYS |
| C-BETA-2 [D2] | — | 112C | — | 227 | P | PRO |
| C-BETA-2 [D2] | — | 113C | — | 228 | V | VAL |
| C-BETA-2 [D2] | — | 114C | — | 229 | T | THR |
| C-BETA-2 [D2] | — | 115C | — | 230 | Q | GLN |
| C-BETA-2 [D2] | — | 116C | — | 231 | I | ILE |
| C-BETA-2 [D2] | — | 117C | — | 232 | V | VAL |
| C-BETA-2 [D2] | — | 118C | — | 233 | S | SER |
| C-BETA-2 [D2] | — | 119C | — | 234 | A | ALA |
| C-BETA-2 [D2] | — | 120C | — | 235 | E | GLU |
| C-BETA-2 [D2] | — | 121C | — | 236 | A | ALA |
| C-BETA-2 [D2] | — | 122C | — | 237 | W | TRP |
| C-BETA-2 [D2] | — | 123C | — | 238 | G | GLY |
| C-BETA-2 [D2] | — | 124C | — | 239 | R | ARG |
| C-BETA-2 [D2] | — | 125C | — | 240 | A | ALA |
| C-BETA-2 [D2] | — | 126C | — | 241 | D | ASP |

### REFERENCES

1 Savage, P., Stebbing, J., Bower, M. & Crook, T. Why does cytotoxic chemotherapy cure only some cancers? Nature Clinical Practice Oncology 6, 43-52 (2009).
2 Crews, C. M. Targeting the undruggable proteome: the small molecules of my dreams. Chem Biol 17, 551-555, doi: 10.1016/j.chembiol.2010.05.011 (2010).
3 Scott, A. M., Wolchok, J. D. & Old, L. J. Antibody therapy of cancer. Nature Reviews Cancer 12, 278-287, doi: 10.1038/nrc3236 (2012).
4 Dang, C. V., Reddy, E. P., Shokat, K. M. & Soucek, L. Drugging the ‘undruggable’ cancer targets. Nature Reviews Cancer 17, 502 (2017).
5 He, Q. et al. TCR-like antibodies in cancer immunotherapy. Journal of Hematology & Oncology 12, 99, doi: 10.1186/s13045-019-0788-4 (2019).
6 Chruściel, E. et al. Adoptive Cell Therapy—Harnessing Antigen-Specific T Cells to Target Solid Tumours. Cancers 12, 683 (2020).
7 Wong, W. K., Leem, J. & Deane, C. M. Comparative analysis of the CDR loops of antigen receptors. Frontiers in immunology 10, 2454 (2019).
8 Sewell, A. K. Why must T cells be cross-reactive? Nature Reviews Immunology 12, 669-677 (2012).
9 Leng, Q., Tarbe, M., Long, Q. & Wang, F. Pre-existing heterologous T-cell immunity and neoantigen immunogenicity. Clinical & Translational Immunology 9, e01111 (2020).
10 Kaufman, E. N. & Jain, R. K. Effect of bivalent interaction upon apparent antibody affinity: experimental confirmation of theory using fluorescence photobleaching and implications for antibody binding assays. Cancer research 52, 4157-4167 (1992).
11 Mazor, Y. et al. Enhanced tumor-targeting selectivity by modulating bispecific antibody binding affinity and format valence. Sci Rep 7, 40098-40098, doi: 10.1038/srep40098 (2017).
12 Slaga, D. et al. Avidity-based binding to HER2 results in selective killing of HER2-overexpressing cells by anti-HER2/CD3. Science translational medicine 10 (2018).
13 Salzer, B. et al. Engineering AvidCARs for combinatorial antigen recognition and reversible control of CAR function. Nature Communications 11, 4166, doi: 10.1038/s41467-020-17970-3 (2020).
14 Zhang, C., Zhuang, Q., Liu, J. & Liu, X. Synthetic Biology in Chimeric Antigen Receptor T (CAR T) Cell Engineering. ACS Synthetic Biology 11, 1-15, doi: 10.1021/acssynbio.1c00256 (2022).
15 Roybal, Kole T. et al. Precision Tumor Recognition by T Cells With Combinatorial Antigen-Sensing Circuits. Cell 164, 770-779, doi: https://doi.org/10.1016/j.cell.2016.01.011 (2016).
16 Jorritsma, A. et al. Selecting highly affine and well-expressed TCRs for gene therapy of melanoma. Blood 110, 3564-3572, doi: 10.1182/blood-2007-02-075010 (2007).
17 van Loenen, M. M. et al. Mixed T cell receptor dimers harbor potentially harmful neoreactivity. Proceedings of the National Academy of Sciences 107, 10972-10977 (2010).
18 Rohl, C. A., Strauss, C. E., Misura, K. M. & Baker, D. in Methods Enzymol Vol. 383 66-93 (Elsevier, 2004).
19 Leaver-Fay, A. et al. ROSETTA3: an object-oriented software suite for the simulation and design of macromolecules. Methods Enzymol 487, 545-574, doi: 10.1016/B978-0-12-381270-4.00019-6 (2011).
20 Baker, D. What has de novo protein design taught us about protein folding and biophysics? Protein Science 28, 678-683 (2019).
21 Kuhlman, B. & Bradley, P. Advances in protein structure prediction and design. Nature Reviews Molecular Cell Biology 20, 681-697 (2019).
22 Lewis, S. M. et al. Generation of bispecific IgG antibodies by structure-based design of an orthogonal Fab interface. Nature biotechnology 32, 191-198 (2014).
23 Leaver-Fay, A. et al. Computationally designed bispecific antibodies using negative state repertoires. Structure 24, 641-651 (2016).
24 Shcherbinin, D. S., Belousov, V. A. & Shugay, M. Comprehensive analysis of structural and sequencing data reveals almost unconstrained chain pairing in TCRalphabeta complex. PLOS Comput Biol 16, e1007714, doi: 10.1371/journal.pcbi. 1007714 (2020).
25 Wu, X. et al. in MAbs. 470-482 (Taylor & Francis).
26 Froning, K. et al. Computational stabilization of T cell receptors allows pairing with antibodies to form bispecifics. Nature communications 11, 1-14 (2020).
27 Parray, H. A. et al. Hybridoma technology a versatile method for isolation of monoclonal antibodies, its applicability across species, limitations, advancement and future perspectives. International immunopharmacology 85, 106639 (2020).
28 Ma, P. et al. Avidity-Based Selection of Tissue-Specific CAR-T Cells from a Combinatorial Cellular Library of CARs. Advanced Science 8, 2003091 (2021).
29 Hartmann, J., Schüβler-Lenz, M., Bondanza, A. & Buchholz, C. J. Clinical development of CAR T cells-challenges and opportunities in translating innovative treatment concepts. EMBO Molecular Medicine 9, 1183-1197, doi: https://doi.org/10.15252/emmm.201607485 (2017).
30 Labanieh, L., Majzner, R. G. & Mackall, C. L. Programming CAR-T cells to kill cancer. Nature biomedical engineering 2, 377-391 (2018).
31 Ambroggio, X. I. & Kuhlman, B. Computational Design of a Single Amino Acid Sequence that Can Switch between Two Distinct Protein Folds. Journal of the American Chemical Society 128, 1154-1161, doi: 10.1021/ja054718w (2006).
32 Leaver-Fay, A., Jacak, R., Stranges, P. B. & Kuhlman, B. A generic program for multistate protein design. PloS one 6, e20937 (2011).
33 Parrish, J., Metters, H., Chen, L. & Xue, D. Demonstration of the in vivo interaction of key cell death regulators by structure-based design of second-site suppressors. Proceedings of the National Academy of Sciences 97, 11916-11921 (2000).
34 Kortemme, T. et al. Computational redesign of protein-protein interaction specificity. Nature structural & molecular biology 11, 371-379 (2004).
35 Sammond, D. W., Eletr, Z. M., Purbeck, C. & Kuhlman, B. Computational design of second-site suppressor mutations at protein-protein interfaces. Proteins: Structure, Function, and Bioinformatics 78, 1055-1065 (2010).
36 van Boxel, G. I. et al. Some lessons from the systematic production and structural analysis of soluble αβ T-cell receptors. Journal of immunological methods 350, 14-21 (2009).
37 Stone, J. D., Chervin, A. S., Aggen, D. H. & Kranz, D. M. in Methods Enzymol Vol. 503 189-222 (Elsevier, 2012).
38 Govers, C., Sebestyén, Z., Coccoris, M., Willemsen, R. A. & Debets, R. T cell receptor gene therapy: strategies for optimizing transgenic TCR pairing. Trends in Molecular Medicine 16, 77-87, doi: https://doi.org/10.1016/j.molmed.2009.12.004 (2010).
39 Chandran, S. S. & Klebanoff, C. A. T cell receptor-based cancer immunotherapy: emerging efficacy and pathways of resistance. Immunological reviews 290, 127-147 (2019).
40 Zhang, J. & Wang, L. The emerging world of TCR-T cell trials against cancer: a systematic review. Technology in cancer research & treatment 18, 1533033819831068 (2019).
41 Froning, K. J. et al. Computational design of a specific heavy chain/kappa light chain interface for expressing fully IgG bispecific antibodies. Protein Sci 26, 2021-2038, doi: 10.1002/pro.3240 (2017).
42 Borbulevych, O. Y., Santhanagopolan, S. M., Hossain, M. & Baker, B. M. TCRs used in cancer gene therapy cross-react with MART-1/Melan-A tumor antigens via distinct mechanisms. The Journal of Immunology 187, 2453-2463 (2011).
43 Emsley, P., Lohkamp, B., Scott, W. G. & Cowtan, K. Features and development of Coot. Acta Crystallographica Section D: Biological Crystallography 66, 486-501 (2010).
44 Terwilliger, T. C. et al. Iterative model building, structure refinement and density modification with the PHENIX AutoBuild wizard. Acta Crystallographica Section D: Biological Crystallography 64, 61-69 (2008).
45 Leisegang, M., Kammertoens, T., Uckert, W. & Blankenstein, T. Targeting human melanoma neoantigens by T cell receptor gene therapy. The Journal of clinical investigation 126, 854-858 (2016).
46 Bargh, J. D., Isidro-Llobet, A., Parker, J. S. & Spring, D. R. Cleavable linkers in antibody-drug conjugates. Chemical Society Reviews 48, 4361-4374 (2019).
47 Khongorzul, P., Ling, C. J., Khan, F. U., Ihsan, A. U. & Zhang, J. Antibody-drug conjugates: a comprehensive review. Molecular Cancer Research 18, 3-19 (2020).
48 Park, H. I., Yoon, H. W. & Jung, S. T. The highly evolvable antibody Fc domain. Trends in biotechnology 34, 895-908 (2016).
49 Kang, T. H. & Jung, S. T. Boosting therapeutic potency of antibodies by taming Fc domain functions. Experimental & molecular medicine 51, 1-9 (2019).
50 Baeuerle, P. A. & Reinhardt, C. Bispecific T-cell engaging antibodies for cancer therapy. Cancer research 69, 4941-4944 (2009).
51 Huehls, A. M., Coupet, T. A. & Sentman, C. L. Bispecific T-cell engagers for cancer immunotherapy. Immunology and cell biology 93, 290-296 (2015).
52 Mamedov, T. et al. Production of non-glycosylated recombinant proteins in Nicotiana benthamiana plants by co-expressing bacterial PNGase F. Plant biotechnology journal 10, 773-782 (2012).
53 Brea, E. J. et al. Kinase Regulation of Human MHC Class I Molecule Expression on Cancer Cells. Cancer Immunol Res 4, 936-947, doi: 10.1158/2326-6066.CIR-16-0177 (2016).
54 Ong, C. E., Lyons, A. B., Woods, G. M. & Flies, A. S. Inducible IFN-γ expression for MHC-I upregulation in devil facial tumor cells. Frontiers in immunology 9, 3117 (2019).
55 Pule, M. A. et al. Virus-specific T cells engineered to coexpress tumor-specific receptors: persistence and antitumor activity in individuals with neuroblastoma. Nature medicine 14, 1264-1270 (2008).
56 Pulè, M. A. et al. A chimeric T cell antigen receptor that augments cytokine release and supports clonal expansion of primary human T cells. Molecular Therapy 12, 933-941 (2005).
57 Perna, S. K. et al. Interleukin-7 mediates selective expansion of tumor-redirected cytotoxic T lymphocytes (CTLs) without enhancement of regulatory T-cell inhibition. Clinical Cancer Research 20, 131-139 (2014).
58 Krenciute, G. et al. Transgenic expression of IL15 improves antiglioma activity of IL13Rα2-CAR T cells but results in antigen loss variants. Cancer Immunol Res 5, 571-581 (2017).
59 Hirabayashi, K. et al. Dual-targeting CAR-T cells with optimal co-stimulation and metabolic fitness enhance antitumor activity and prevent escape in solid tumors. Nature Cancer 2, 904-918, doi: 10.1038/s43018-021-00244-2 (2021).
60 Williams, J. Z. et al. Precise T cell recognition programs designed by transcriptionally linking multiple receptors. Science 370, 1099-1104 (2020).
61 Choe, J. H. et al. SynNotch-CAR T cells overcome challenges of specificity, heterogeneity, and persistence in treating glioblastoma. Science translational medicine 13, eabe 7378 (2021).
62 Minagawa, A. et al. Enhancing T cell receptor stability in rejuvenated iPSC-derived T cells improves their use in cancer immunotherapy. Cell Stem Cell 23, 850-858. e854 (2018).
63 Biernacki, M. A., Brault, M. & Bleakley, M. TCR-based Immunotherapy for hematologic malignancies. Cancer journal (Sudbury, Mass.) 25, 179 (2019).
64 Mitsunaga, M. et al. Cancer cell-selective in vivo near infrared photoimmunotherapy targeting specific membrane molecules. Nature Medicine 17, 1685-1691, doi: 10.1038/nm.2554 (2011).

The foregoing is illustrative of the present invention, and is not to be construed as limiting thereof. The invention is defined by the following claims, with equivalents of the claims to be included therein.

That which is claimed is:

1. A synthetic T-cell receptor (TCR) molecule comprising:
a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen,
wherein the TCRCα and TCRCβ or fragments thereof are modified TCRCα and TCRCβ or fragments thereof wherein the first and the second interface are bioorthogonal to each other, and/or wherein the TCRVα and TCRVβ or fragments thereof are modified TCRVα and TCRVβ or fragments thereof wherein the third and the fourth interface are bioorthogonal to each other;
wherein the bioorthogonal first and second interface and/or the bioorthogonal third and fourth interface each comprise one or more amino acid substitutions,
wherein the bioorthogonal first and second interface selectively bind to each other and/or the bioorthogonal third and fourth interface selectively bind to each other via the one or more substitutions of each interface (e.g., wherein the bioorthogonal TCRC interfaces and/or bioorthogonal TCRV interfaces selectively bind to each other, e.g., selectively bind to each other as compared to unmodified interfaces).

2. The synthetic TCR molecule of claim 1, comprising
the modified TCRCα or fragment thereof with a first interface and the modified TCRCβ or fragment thereof with a second interface which is bioorthogonal to the first interface; and
the modified TCRVα or fragment thereof with a third interface, and the modified TCRVβ or fragment thereof with a fourth interface which is bioorthogonal to the third interface.

3. The synthetic TCR molecule of claim 1 or 2, wherein each of the (modified and/or unmodified) TCRCα, TCRCβ, TCRVα, and TCRVα domains or fragment thereof comprises an N-terminus and a C-terminus, and wherein the TCRCα C-terminus is linked to the TCRVα N-terminus, and wherein the TCRCβ C-terminus is linked to the TCRVβ N-terminus.

4. The synthetic TCR molecule any one of claims 1-3, wherein the one or more substitutions of the bioorthogonal first interface comprise substitution at amino acid position 124 (Kabat 122), 145, 171, 172, 175, 177, 179, and/or 205, wherein the numbering corresponds to PDB numbering of the reference TCRCα amino acid sequence of PDB: 6U07_A (e.g., PDB numbering within residues 118-213 of the reference TCRCα amino acid sequence of 6U07_A (Chain A) in the Protein Data Bank (rcsb.org/sequence/6U07)).

5. The synthetic TCR molecule of claim 4, wherein the one or more substitutions of the bioorthogonal first interface comprise 124F, 124Q, 124R (Kabat 122F, 122Q, 122R), 145H, 171Q, 172D, 175R, 177K, 179R, and/or 205K.

6. The synthetic TCR molecule of claim 5, wherein the one or more substitutions of the bioorthogonal first interface comprise D124F, D124Q, D124R (Kabat D122F, D122Q, D122R), D145H, R171Q, S172D, F175R, S177K, S179R, and/or F205K.

7. The synthetic TCR molecule of any one of claims 1-6, wherein the one or more substitutions of the bioorthogonal second interface comprise substitution at amino acid position 139, 142, 170, 195 and/or, 197, wherein the numbering corresponds to PDB numbering of the reference TCRCβ amino acid sequence of PDB: 6U07_B (e.g., PDB numbering within residues 117-247 of the reference TCRCβ amino acid sequence of 6U07_B (Chain B) in the Protein Data Bank (rcsb.org/sequence/6U07)).

8. The synthetic TCR molecule of claim 7, wherein the one or more substitutions of the bioorthogonal second interface comprise 139L, 139D, 139E, 142E, 170K, 195T, 195S and/or 197S.

9. The synthetic TCR molecule of claim 8, wherein the one or more substitutions of the bioorthogonal first interface comprise R139L, R139D, R139E, K142E, D170K, R195T, R195S and/or R197S.

10. The synthetic TCR molecule of any one of claims 1-9, wherein the one or more substitutions of the bioorthogonal first interface and the bioorthogonal second interface comprise:
(i) 179R in the first interface and 195S in the second interface;
(ii) 124R and 205K in the first interface and 139E in the second interface;
(iii) 124R, 179R and 205K in the first interface and 139E and 195S in the second interface;
(iv) 124Q and 205K in the first interface and 139L in the second interface;
(v) 179R in the first interface and 195T in the second interface;
(vi) 175R and 177K in the first interface and 142E and 197S in the second interface;
(vii) 124F and 205W in the first interface and 139L in the second interface;
(viii) 171Q and 172D in the first interface and 170K in the second interface;
(ix) 145H, 175R and 177K in the first interface and 142E and 197S in the second interface;
(x) 124R and 205K in the first interface and 139D in the second interface;
(xi) 124Q, 179R, and 205K in the first interface and 139L and 195T in the second interface;
(xii) 124Q, 145H, 185R, 177K, 179R and 205K in the first interface and 139L, 142E, 195T and 197S in the second interface;
(xiii) 124R, 179R and 205K in the first interface and 139D and 195S in the second interface, and/or
(xiv) 124Q, 175R, 177K, 179R, and 205K in the first interface and 139L, 142E, 195T and 197S in the second interface.

11. The synthetic TCR molecule of claim 10, wherein the modified TCRCα domain and modified TCRCβ domain or fragment thereof comprises the amino acid sequences of:
(i)
(TCRCα; SEQ ID NO: 3)
PYIQNPDPAVYQLRDSKSSDKFVCLFTDFDSQINVSQSKDSDVYITDKC
VLDMRSMDFKSNRAVAWSNKSDFTCANAFNNSIIPEDTFFPSPESSC,
and
(TCRCβ; SEQ ID NO: 4)
EDLKNVFPPEVAVFEPSKAEISRTQKATLVCLATGFYPPHVELSWWVNG
KEVHDGVCTDPQPLKEQPALNDSRYALSSSLRVSATFWQDPRNHFRCQV
QFYGLSENDEWTQDRAKPVTQIVSAEAWGRADC;
(ii)
(TCRCα; SEQ ID NO: 5)
PYIQNPRPAVYQLRDSKSSDKFVCLFTDFDSQINVSQSKDSDVYITDKC
VLDMRSMDFKSNSAVAWSNKSDFTCANAFNNSIIPEDTKFPSPESSC,
and
(TCRCβ; SEQ ID NO: 6)
EDLKNVFPPEVAVFEPSKAEISETQKATLVCLATGFYPPHVELSWWVNG
KEVHDGVCTDPQPLKEQPALNDSRYALSSRLRVSATFWQDPRNHFRCQV
QFYGLSENDEWTQDRAKPVTQIVSAEAWGRADC;
(iii)
(TCRCα; SEQ ID NO: 7)
PYIQNPRPAVYQLRDSKSSDKFVCLFTDFDSQINVSQSKDSDVYITDKC
VLDMRSMDFKSNRAVAWSNKSDFTCANAFNNSIIPEDTKFPSPESSC,
and
(TCRCβ; SEQ ID NO: 8)
EDLKNVFPPEVAVFEPSKAEISETQKATLVCLATGFYPPHVELSWWVNG
KEVHDGVCTDPQPLKEQPALNDSRYALSSSLRVSATFWQDPRNHFRCQV
QFYGLSENDEWTQDRAKPVTQIVSAEAWGRADC;
(iv)
(TCRCα; SEQ ID NO: 9)
PYIQNPQPAVYQLRDSKSSDKFVCLFTDFDSQINVSQSKDSDVYITDKC
VLDMRSMDFKSNSAVAWSNKSDFTCANAFNNSIIPEDTKFPSPESSC,
and
(TCRCβ; SEQ ID NO: 10)
EDLKNVFPPEVAVFEPSKAEISLTQKATLVCLATGFYPPHVELSWWVNG
KEVHDGVCTDPQPLKEQPALNDSRYALSSRLRVSATFWQDPRNHFRCQV
QFYGLSENDEWTQDRAKPVTQIVSAEAWGRADC;
(v)
(TCRCα; SEQ ID NO: 11)
PYIQNPDPAVYQLRDSKSSDKFVCLFTDFDSQINVSQSKDSDVYITDKC
VLDMRSMDFKSNRAVAWSNKSDFTCANAFNNSIIPEDTFFPSPESSC,
and
(TCRCβ; SEQ ID NO: 12)
EDLKNVFPPEVAVFEPSKAEISRTQKATLVCLATGFYPPHVELSWWVNG
KEVHDGVCTDPQPLKEQPALNDSRYALSSTLRVSATFWQDPRNHFRCQV
QFYGLSENDEWTQDRAKPVTQIVSAEAWGRADC;
(vi)
(TCRCα; SEQ ID NO: 13)
PYIQNPDPAVYQLRDSKSSDKFVCLFTDFDSQINVSQSKDSDVYITDKC
VLDMRSMDRKKNSAVAWSNKSDFTCANAFNNSIIPEDTFFPSPESSC,
and
(TCRCβ; SEQ ID NO: 14)
EDLKNVFPPEVAVFEPSKAEISRTQEATLVCLATGFYPPHVELSWWVNG
KEVHDGVCTDPQPLKEQPALNDSRYALSSRLSVSATFWQDPRNHFRCQV
QFYGLSENDEWTQDRAKPVTQIVSAEAWGRADC;
(vii)
(TCRCα; SEQ ID NO: 15)
PYIQNPFPAVYQLRDSKSSDKFVCLFTDFDSQINVSQSKDSDVYITDKC
VLDMRSMDFKSNSAVAWSNKSDFTCANAFNNSIIPEDTWFPSPESSC,
and
(TCRCβ; SEQ ID NO: 16)
EDLKNVFPPEVAVFEPSKAEISLTQKATLVCLATGFYPPHVELSWWVNG
KEVHDGVCTDPQPLKEQPALNDSRYALSSRLRVSATFWQDPRNHFRCQV
QFYGLSENDEWTQDRAKPVTQIVSAEAWGRADC;
(viii)
(TCRCα: SEQ ID NO: 17)
PYIQNPDPAVYQLRDSKSSDKFVCLFTDFDSQINVSQSKDSDVYITDKC
VLDMQDMDFKSNSAVAWSNKSDFTCANAFNNSIIPEDTFFPSPESSC,
and
(TCRCβ; SEQ ID NO: 18)
EDLKNVFPPEVAVFEPSKAEISRTQKATLVCLATGFYPPHVELSWWVNG
KEVHKGVCTDPQPLKEQPALNDSRYALSSRLRVSATFWQDPRNHFRCQV
QFYGLSENDEWTQDRAKPVTQIVSAEAWGRADC;
(ix)
(TCRCα; SEQ ID NO: 19)
PYIQNPDPAVYQLRDSKSSDKFVCLFTHFDSQINVSQSKDSDVYITDKC
VLDMRSMDRKKNSAVAWSNKSDFTCANAFNNSIIPEDTFFPSPESSC,
and
(TCRCβ; SEQ ID NO: 20)
EDLKNVFPPEVAVFEPSKAEISRTQEATLVCLATGFYPPHVELSWWVNG
KEVHDGVCTDPQPLKEQPALNDSRYALSSRLSVSATFWQDPRNHFRCQV
QFYGLSENDEWTQDRAKPVTQIVSAEAWGRADC;
(x)
(TCRCα; SEQ ID NO: 21)
PYIQNPRPAVYQLRDSKSSDKFVCLFTDFDSQINVSQSKDSDVYITDKC
VLDMRSMDFKSNSAVAWSNKSDFTCANAFNNSIIPEDTKFPSPESSC,
and
(TCRCβ; SEQ ID NO: 22)
EDLKNVFPPEVAVFEPSKAEISDTQKATLVCLATGFYPPHVELSWWVNG
KEVHDGVCTDPQPLKEQPALNDSRYALSSRLRVSATFWQDPRNHFRCQV
QFYGLSENDEWTQDRAKPVTQIVSAEAWGRADC;
(xi)
(TCRCα; SEQ ID NO: 23)
PYIQNPQPAVYQLRDSKSSDKFVCLFTDFDSQINVSQSKDSDVYITDKC
VLDMRSMDFKSNRAVAWSNKSDFTCANAFNNSIIPEDTKFPSPESSC,
and
(TCRCβ; SEQ ID NO: 24)
EDLKNVFPPEVAVFEPSKAEISLTQKATLVCLATGFYPPHVELSWWVNG
KEVHDGVCTDPQPLKEQPALNDSRYALSSTLRVSATFWQDPRNHFRCQV
QFYGLSENDEWTQDRAKPVTQIVSAEAWGRADC;
(xii)
(TCRCα; SEQ ID NO: 25)
PYIQNPQPAVYQLRDSKSSDKFVCLFTHFDSQINVSQSKDSDVYITDKC
VLDMRSMDRKKNRAVAWSNKSDFTCCANAFNNSIIPEDTKFPSPESSC,
and
(TCRCβ; SEQ ID NO: 26)
EDLKNVFPPEVAVFEPSKAEISLTQEATLVCLATGFYPPHVELSWWVNG
KEVHDGVCTDPQPLKEQPALNDSRYALSSTLSVSATFWQDPRNHFRCQV
QFYGLSENDEWTQDRAKPVTQIVSAEAWGRADC;
(xiii)
(TCRCα; SEQ ID NO: 27)
PYIQNPRPAVYQLRDSKSSDKFVCLFTDFDSQINVSQSKDSDVYITDKC
VLDMRSMDFKSNRAVAWSNKSDFTCANAFNNSIIPEDTKFPSPESSC,
and
(TCRCBβ; SEQ ID NO: 28)
EDLKNVFPPEVAVFEPSKAEISDTQKATLVCLATGFYPPHVELSWWVNG
KEVHDGVCTDPQPLKEQPALNDSRYALSSSLRVSATFWQDPRNHFRCQV
QFYGLSENDEWTQDRAKPVTQIVSAEAWGRADC;
and/or
(xiv)
(TCRCα; SEQ ID NO: 29)
PYIQNPQPAVYQLRDSKSSDKFVCLFTDFDSQINVSQSKDSDVYITDKC
VLDMRSMDRKKNRAVAWSNKSDFTCANAFNNSIIPEDTKFPSPESSC,
and
(TCRCβ; SEQ ID NO: 30)
EDLKNVFPPEVAVFEPSKAEISLTQEATLVCLATGFYPPHVELSWWVNG
KEVHDGVCTDPQPLKEQPALNDSRYALSSTLSVSATFWQDPRNHFRCQV
QFYGLSENDEWTQDRAKPVTQIVSAEAWGRADC.

12. The synthetic TCR molecule of any one of claims 1-11, wherein the one or more amino acid substitutions of each the bioorthogonal third and fourth interface are comprised in a conserved region of each of the modified TCRVα or fragment thereof and modified TCRVβ or fragment thereof.

13. The synthetic TCR molecule of claim 12, wherein the conserved region of each of the modified TCRVα or fragment thereof and modified TCRVβ or fragment thereof comprises amino acid Kabat positions 1-24, 32-48, 62-92, and/or 105-116 in the TCRVα or fragment thereof, and/or amino acid Kabat positions 1-24, 32-48, 65-94, and/or 107-116 in the TCRVβ or fragment thereof.

14. The synthetic TCR molecule of any one of claims 1-13, wherein the one or more substitutions of the bioorthogonal third interface comprise substitution at amino acid positions 37 and/or 108 (Kabat 109), wherein the numbering corresponds to PDB numbering of the reference TCRVα amino acid sequence of PDB: 2F53_D (e.g., PDB numbering within residues-1 to 191 of the reference TCRVα amino acid sequence of 2F53_D (Chain D) in the Protein Data Bank (rcsb.org/sequence/2F53)).

15. The synthetic TCR molecule of claim 14, wherein the one or more substitutions of the bioorthogonal third interface comprise 37Y, 37K, 37D, 37L, 37V, or 108K (Kabat 109K).

16. The synthetic TCR molecule of claim 15, wherein the one or more substitutions of the bioorthogonal third interface comprise Q37Y, Q37K, Q37D, Q37L, Q37V, or S108K (Kabat S109K).

17. The synthetic TCR molecule of any one of claims 1-16, wherein the one or more substitutions of the bioorthogonal fourth interface comprise substitution at amino acid position 35 (Kabat 37) and/or 38 (Kabat 40) wherein the numbering corresponds to PDB numbering of the reference TCRVα amino acid sequence of PDB: 2F53_E (e.g., PDB numbering within residues-1 to 241 of the reference TCRVβ amino acid sequence of 2F53_E (Chain E) in the Protein Data Bank (rcsb.org/sequence/2F53)).

18. The synthetic TCR molecule of claim 17, wherein the one or more substitutions of the bioorthogonal fourth interface comprise 35K, 35Y, 35D, 35M (Kabat 37K, 37Y, 37D, 37M), or 38E (Kabat 40E).

19. The synthetic TCR molecule of claim 18, wherein the one or more substitutions of the bioorthogonal fourth interface comprise Q35K, Q35Y, Q35D, Q35M, (Kabat Q37K, Q37Y, Q37D, Q37M), or G38E (Kabat G40E).

20. The synthetic TCR molecule of any one of claims 1-19, wherein the one or more substitutions of the bioorthogonal third interface and the bioorthogonal fourth interface comprise:
(i) 37K in the third interface and 35Y (Kabat 37Y) in the fourth interface;
(ii) 37K in the third interface and 35Y (Kabat 37Y) in the fourth interface;
(iii) 37K in the third interface and 35D (Kabat 37D) in the fourth interface;
(iv) 37D in the third interface and 35K (Kabat 37K) in the fourth interface;
(v) 37V in the third interface and 35M (Kabat 37M) in the fourth interface;
(vi) 108K (Kabat 109K) in the third interface and 38E (Kabat 40E) in the fourth interface; and/or
(vii) 37L and 35M (Kabat 37M) in the third interface and 139E in the fourth interface.

21. The synthetic TCR molecule of claim 20, wherein the modified TCRVα domain and modified TCRVβ domain or fragment thereof comprises the amino acid sequences of:
(i)
(TCRVα; SEQ ID NO: 33)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 34)
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL;
(ii)
(TCRVα; SEQ ID NO: 35)
QEVTQIPAALSVPEGENLVLNCSFTDSAIYNLQWFRYDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 36)
GVTQTPKFQVLKTGQSMTLQCAQDMNHEYMSWYRKDPGMGLRLIHYSVA
IQTTDQGEVPNGYNVSRSTIEDFPLRLLSAAPSQTSVYFCASSYVGDTG
ELFFGEGSRLTVL;
(iii)
(TCRVα; SEQ ID NO: 37)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 38)
GVTQTPKFQVLKTGQSMTLQCAQDMNHEYMSWYRDDPGMGLRLIHYSVA
IQTTDQGEVPNGYNVSRSTIEDFPLRLLSAAPSQTSVYFCASSYVGDTG
ELFFGEGSRLTVL;
(iv)
(TCRVα; SEQ ID NO: 39)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 40)
GVTQTPKFQVLKTGQSMTLQCAQDMNHEYMSWYRKDPGMGLRLIHYSVA
IQTTDQGEVPNGYNVSRSTIEDFPLRLLSAAPSQTSVYFCASSYVGDTG
ELFFGEGSRLTVL;
(v)
(TCRVα; SEQ ID NO: 41)
QEVTQIPAALSVPEGENLVLNCSFTDSAIYNLQWFRVDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 42)
GVTQTPKFQVLKTGQSMTLQCAQDMNHEYMSWYRMDPGMGLRLIHYSVA
IQTTDQGEVPNGYNVSRSTIEDFPLRLLSAAPSQTSVYFCASSYVGDTG
ELFFGEGSRLTVL;
(vi)
(TCRVα; SEQ ID NO: 43)
QEVTQIPAALSVPEGENLVLNCSFTDSAIYNLQWFRQDPGKGLTSLLLI
SPWQREQTSGRLNASLDKSSGRSTLYIAASQPGDSATYLCAVRPLLDGT
YIPTFGRGTKLIVH,
and
(TCRVβ; SEQ ID NO: 44)
GVTQTPKFQVLKTGQSMTLQCAQDMNHEYMSWYRQDPEMGLRLIHYSVA
IQTTDQGEVPNGYNVSRSTIEDFPLRLLSAAPSQTSVYFCASSYVGDTG
ELFFGEGSRLTVL;
and/or
(vii)
(TCRVα; SEQ ID NO: 45)
QEVTQIPAALSVPEGENLVLNCSFTDSAIYNLQWFRLDPGKGLTSLLLI
SPWQREQTSGRLNASLDKSSGRSTLYIAASQPGDSATYLCAVRPLLDGT
YIPTFGRGTSLIVH,
and
(TCRVβ; SEQ ID NO: 46)
GVTQTPKFQVLKTGQSMTLQCAQDMNHEYMSWYRMDPGMGLRLIHYSVA
IQTTDQGEVPNGYNVSRSTIEDFPLRLLSAAPSQTSVYFCASSYVGDTG
ELFFGEGSRLTVL.

22. The synthetic TCR molecule of any one of claims 1-21, wherein the one or more substitutions of each bioorthogonal interface comprise:
(i) 124D (Kabat 122D), 179R, and 205K in the first interface, 139L and 195T in the second interface, 37K in the third interface, and 35Y (Kabat 37Y) in the fourth interface;
(ii) 124Q (Kabat 122Q), 175R, 177K, 179S and 205K in the first interface, 139L, 142E, 195T and 197S in the second interface, 37K in the third interface, and 35Y (Kabat 37Y) in the fourth interface;
(iii) 124Q (Kabat 122Q), 145H, 175R, 177K, 179R and 205K in the first interface, 139L, 142E, 195T and 197S in the second interface, 37K in the third interface, and 35Y (Kabat 37Y) in the fourth interface;
(iv) 124R (Kabat 122R), 179R and 205K in the first interface, 139D and 195S in the second interface, 37K in the third interface, and 35Y (Kabat 37Y) in the fourth interface; and/or
(v) 124Q (Kabat 122Q), 179S and 205K in the first interface, 139E and 195S in the second interface, 37K in the third interface, and 35Y (Kabat 37Y) in the fourth interface.

23. The synthetic TCR molecule of claim 22, wherein the modified TCRCα domain, modified TCRCβ domain, TCRVα domain, modified TCRVβ domain, or fragments thereof comprise the amino acid sequences of:
(i)
(SEQ ID NOs: 23, 24, 33 and 34)
PYIQNPQPAVYQLRDSKSSDKFVCLFTDFDSQINVSQSKDSDVYITDKC
VLDMRSMDFKSNRAVAWSNKSDFTCANAFNNSIIPEDTKFPSPESSC
(TCRCα),
EDLKNVFPPEVAVFEPSKAEISLTQKATLVCLATGFYPPHVELSWWVNG
KEVHDGVCTDPQPLKEQPALNDSRYALSSTLRVSATFWQDPRNHFRCQV
QFYGLSENDEWTQDRAKPVTQIVSAEAWGRADC (TCRCβ)
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH (TCRVα),
and
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL (TCRVβ);
(ii)
(SEQ ID NOs: 29, 30, 33 and 34)
PYIQNPQPAVYQLRDSKSSDKFVCLFTDFDSQINVSQSKDSDVYITDKC
VLDMRSMDRKKNRAVAWSNKSDFTCANAFNNSIIPEDTKFPSPESSC
(TCRCα),
EDLKNVFPPEVAVFEPSKAEISLTQEATLVCLATGFYPPHVELSWWVNG
KEVHDGVCTDPQPLKEQPALNDSRYALSSTLSVSATFWQDPRNHFRCQV
QFYGLSENDEWTQDRAKPVTQIVSAEAWGRADC (TCRCβ),
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH (TCRVα),
and
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL (TCRVβ);
(iii)
(SEQ ID NOs: 25, 26, 33 and 34)
PYIQNPQPAVYQLRDSKSSDKFVCLFTHFDSQINVSQSKDSDVYITDKC
VLDMRSMDRKKNRAVAWSNKSDFTCCANAFNNSIIPEDTKFPSPESSC
(TCRCα),
EDLKNVFPPEVAVFEPSKAEISLTQEATLVCLATGFYPPHVELSWWVNG
KEVHDGVCTDPQPLKEQPALNDSRYALSSTLSVSATFWQDPRNHFRCQV
QFYGLSENDEWTQDRAKPVTQIVSAEAWGRADC (TCRCβ),
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH (TCRVα),
and
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL (TCRVβ);
(iv)
(SEQ ID NOs: 27, 28, 33 and 34)
PYIQNPRPAVYQLRDSKSSDKFVCLFTDFDSQINVSQSKDSDVYITDKC
VLDMRSMDFKSNRAVAWSNKSDFTCANAFNNSIIPEDTKFPSPESSC
(TCRCα),
EDLKNVFPPEVAVFEPSKAEISDTQKATLVCLATGFYPPHVELSWWVNG
KEVHDGVCTDPQPLKEQPALNDSRYALSSSLRVSATFWQDPRNHFRCQV
QFYGLSENDEWTQDRAKPVTQIVSAEAWGRADC (TCRCβ),
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH (TCRVα),
and
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL (TCRVβ);
and/or
(v)
(SEQ ID NOs: 7, 8, 33 and 34)
PYIQNPRPAVYQLRDSKSSDKFVCLFTDFDSQINVSQSKDSDVYITDKC
VLDMRSMDFKSNRAVAWSNKSDFTCANAFNNSIIPEDTKFPSPESSC
(TCRCα),
EDLKNVFPPEVAVFEPSKAEISETQKATLVCLATGFYPPHVELSWWVNG
KEVHDGVCTDPQPLKEQPALNDSRYALSSSLRVSATFWQDPRNHFRCQV
QFYGLSENDEWTQDRAKPVTQIVSAEAWGRADC (TCRCβ),
QEVTQIPAALSVPEGENLVLNCSFTDSAIYNLQWFRKDPGKGLTSLLLI
SPWQREQTSGRLNASLDKSSGRSTLYIAASQPGDSATYLCAVRPLLDGT
YIPTFGRGTSLIVH (TCRVα),
and
GVTQTPKFQVLKTGQSMTLQCAQDMNHEYMSWYRYDPGMGLRLIHYSVA
IQTTDQGEVPNGYNVSRSTIEDFPLRLLSAAPSQTSVYFCASSYVGDTG
ELFFGEGSRLTVL (TCRVβ).

24. The synthetic TCR molecule of any one of claims 1-23, in soluble form (e.g., wherein the synthetic TCR molecule is devoid of a transmembrane domain).

25. The synthetic TCR molecule of any one of claims 1-24, further comprising a second TCRCα, TCRCβ, TCRVα, and TCRVβ or fragments thereof, each comprising an N-terminus and a C-terminus and a first, second, third and fourth interface, respectively, wherein at least a portion of each of the second TCRVα and TCRVβ together form a variable portion with binding specificity to a second target antigen that is different from the first target antigen.

26. The synthetic TCR molecule of claim 25, wherein the second TCRCα, TCRCβ, TCRVα, and TCRVβ or fragments thereof are unmodified (e.g., wildtype).

27. The synthetic TCR molecule of claim 25, wherein the second TCRCα, TCRCβ, TCRVα, and TCRVβ or fragments thereof are modified (e.g., modified to comprise bioorthogonal interfaces, e.g., wherein the second TCRCα and TCRCβ first and second interfaces selectively bind to each other and/or the second TCRVα and TCRVβ third and fourth interfaces selectively bind to each other, e.g., selectively bind to each other as compared to unmodified interfaces).

28. The synthetic TCR molecule of any one of claims 25-27, wherein the C-terminus or N-terminus of the modified TCRCα, TCRCβ, TCRVα, and/or TCRVβ or fragment thereof with an interface which is bioorthogonal to a corresponding interface, is linked to the N-terminus or C-terminus of the second (e.g., modified or unmodified) TCRCα, TCRCβ, TCRVα, and/or TCRVβ or fragment thereof.

29. The synthetic TCR molecule of any one of claims 25-27, further comprising an antibody Fc or fragment thereof comprising an N-terminus and a C-terminus, wherein the C-terminus of the modified TCRCα or fragment thereof with a first interface which is bioorthogonal to the second interface, or the C-terminus of the TCRCβ or fragment thereof with a second interface which is bioorthogonal to the first interface, is linked to an N-terminus of the antibody Fc or fragment thereof, and optionally wherein the C-terminus of the second (modified or unmodified) TCRCα or fragment thereof or the C-terminus of the second TCRCβ or fragment thereof is linked to another N-terminus of the antibody Fc or fragment thereof.

30. The synthetic TCR molecule of claim 29, wherein the antibody Fc or fragment thereof comprises an IgE, IgA, IgM, IgD, or IgG Fc or fragment thereof.

31. The synthetic TCR molecule of claim 29 or 30, wherein the antibody Fc or fragment thereof comprises two or more bioorthogonal domains, each comprising one or more modifications (e.g., amino acid substitutions) and which selectively bind to each other via the one or more modifications (e.g., wherein the Fc domains are bioorthogonal, e.g., selectively bind to each other as compared to unmodified interfaces).

32. The synthetic TCR molecule of any one of claims 25-27, further comprising an antibody or antibody fragment (e.g., an antibody Fab, a Fab′, a F(ab′)2, a Fv, a disulfide linked Fv, a single chain antibody (scFv), a single domain antibody (dAb), a diabody, a nanobody, and/or an affibody or fragment thereof) e.g., in particular embodiments, an antibody scFv) comprising an N-terminus and a C-terminus, wherein the C-terminus of the modified TCRVβ or fragment thereof with a fourth interface which is bioorthogonal to the third interface, is linked to an N-terminus of the antibody or antibody fragment, and optionally wherein the C-terminus of the second (modified or unmodified) TCRCβ or fragment thereof is linked to another N-terminus of the antibody or antibody fragment.

33. The synthetic TCR molecule of any one of claims 1-23, further comprising a T cell inhibitory domain (e.g., PD-1, ITIM) or fragment thereof.

34. The synthetic TCR molecule of any one of claims 1-23, further comprising a T cell signaling domain (e.g., CD3° C.) or fragment thereof and/or a T cell co-stimulatory domain (e.g., CD28, 4-1BB).

35. The synthetic TCR molecule of claim 34, wherein the synthetic TCR molecule is devoid of a signaling domain or fragment thereof.

36. The synthetic TCR molecule of claim 34, wherein the synthetic TCR molecule is devoid of a costimulatory domain or fragment thereof.

37. The synthetic TCR molecule of claim 34, comprising a T cell signaling domain or fragment thereof and a T cell co-stimulatory domain.

38. The synthetic TCR molecule of claim 37, further comprising a second TCRCα, TCRCβ, TCRVα, and TCRVβ or fragments thereof, each comprising an N-terminus and a C-terminus, wherein at least a portion of each of the second TCRVα and TCRVβ together form a variable portion with binding specificity to a second target antigen that is different from the first target antigen.

39. The synthetic TCR molecule of claim 38, wherein the second TCRCα, TCRCβ, TCRVα, and TCRVβ or fragments thereof are unmodified (e.g., wildtype).

40. The synthetic TCR molecule of claim 38, wherein the second TCRCα, TCRCβ, TCRVα, and TCRVβ or fragments thereof are modified (e.g., modified to comprise bioorthogonal interfaces, e.g., wherein the second TCRCα and TCRCβ first and second interfaces selectively bind to each other and/or the second TCRVα and TCRVβ third and fourth interfaces selectively bind to each other, e.g., selectively bind to each other as compared to unmodified interfaces).

41. The synthetic TCR molecule of any one of claims 38-40, wherein the C-terminus or N-terminus of the modified TCRCα, TCRCβ, TCRVa, and/or TCRVβ or fragment thereof with an interface which is bioorthogonal to a corresponding interface, is linked to the N-terminus or C-terminus of the second (e.g., modified or unmodified) TCRCα, TCRCβ, TCRVα, and/or TCRVβ or fragment thereof.

42. The synthetic TCR molecule of any one of claims 33-41, further comprising a hinge region (e.g., CD8 hinge, CD4 hinge), a transmembrane domain, a linker, a costimulatory domain (e.g., CD28, 4-1BB, etc.) and/or an scFv and/or Fab (e.g., wherein the synthetic TCR molecule is a chimeric antigen receptor (CAR).

43. The synthetic TCR molecule of any one of claims 1-42, wherein the synthetic TCR molecule binds a major histocompatibility complex (MHC) (e.g., in vivo and/or in vitro).

44. The synthetic TCR molecule of any one of claims 1-43, wherein the bioorthogonal first and second interface, when selectively bound to each other, and/or the bioorthogonal third and fourth interface, when selectively bound to each other, have a melting temperature of about 55° C. to about 85° C. Tm (e.g., about 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85° C. or any value or range therein, e.g., about 70 to about 80, e.g., for TCRC designs, e.g., about 60 to about 65, e.g., for TCRV designs, about 62 to about 68, e.g., for TCRVC designs).

45. The synthetic TCR molecule of any one of claims 1-44, further comprising a detectable moiety.

46. The synthetic TCR molecule of any one of claims 1-45, Further comprising an effector molecule selected from the group consisting of a drug, a toxin, a small molecule, a radioactive molecule, a photoactivatable molecule, an antibody, a cytokine, an oncolytic virus, an enzyme, a nanoparticle, a biomaterial, a scaffold and any combination thereof.

47. The synthetic TCR molecule of any one of claims 1-46, wherein the target antigen is a cancer antigen, a virus antigen, and/or a bacterial antigen, or any combination thereof.

48. An isolated cell comprising the synthetic TCR molecule of any one of claims 1-47.

49. The isolated cell of claim 48, further comprising a chimeric antigen receptor (CAR) that is different from the synthetic TCR molecule (e.g., that has specificity for a target antigen that is different from the first and/or second target antigen of the synthetic TCR molecule).

50. An isolated cell comprising the synthetic TCR molecule of any one of claims 33-47, wherein the synthetic TCR molecule is expressed on the surface of the cell.

51. A nucleic acid molecule encoding the synthetic TCR molecule of any one of claims 1-47.

52. A vector (e.g., a virus vector, e.g., a virus-like particle) comprising the nucleic acid molecule of claim 51.

53. An isolated cell comprising the vector of claim 52.

54. The cell of any one of claim 48-50 or 53, wherein the cell is selected from the group consisting of an αβT cell (e.g., a CD4+αβT cell, a CD8+αβT cell), a natural killer (NK) cell, a cytotoxic T lymphocyte (CTL), a regulatory T cell, a natural killer T (NKT) cell, a Th17 cell, a γδT cell, a neutrophil, a macrophage, an artificial cell (e.g., cell-like particle) and any combination thereof.

55. A composition comprising the synthetic TCR molecule of any one of claims 1-47, nucleic acid molecule of claim 51, the vector of claim 52, and/or the isolated cell of any one of claim 48-50, 53 or 54.

56. The composition of claim 55, further comprising a pharmaceutically acceptable carrier, diluent and/or adjuvant (e.g., a pharmaceutical composition, e.g., a pharmaceutical formulation).

57. A method of expressing a synthetic TCR molecule in a cell, comprising contacting the cell with the nucleic acid molecule of claim 51, vector of claim 52, and/or composition of any one of claim 55 or 56.

58. The method of claim 57, wherein the cell is in a subject (e.g., a human patient).

59. A method of treating a disorder in a subject, comprising administering to the subject an effective amount of the synthetic TCR molecule of any one of claims 1-47, nucleic acid molecule of claim 51, the vector of claim 52, isolated cell of any one of claim 48-50, 53 or 54, and/or composition of claim 55 or 56, wherein the synthetic TCR binds an antigen associated with the disorder (e.g., a cancer antigen, a viral antigen, a bacterial antigen, or any combination thereof).

60. The method of claim 59, wherein the disorder is cancer (e.g., melanoma, lymphoma, leukemia, pancreatic cancer), a viral infection, a bacterial infection, an autoimmune disease, cellular senescence, or any combination thereof.

61. A method of producing a synthetic T-cell receptor (TCR) molecule, comprising:
(a) providing a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen;
(b) introducing one or more amino acid substitutions into the first and second interface and/or into the third and fourth interface, wherein the one or more amino acid substitutions modify the first and the second interface and/or the third and fourth interface such that the interfaces are bioorthogonal to each other (e.g., such that the bioorthogonal interfaces selectively bind to each other, e.g., selectively bind to each other as compared to unmodified interfaces);
thereby producing a synthetic TCR molecule (e.g., the synthetic TCR molecule of any one of claims 1-47).

62. A method of enhancing stability (e.g., in vitro and/or in vivo) of a T-cell receptor (TCR) molecule, comprising:
(a) providing a TCR alpha chain constant domain (TCRCα) or fragment thereof with a first interface, a TCR beta chain constant domain (TCRCβ) or fragment thereof with a second interface, a TCR alpha chain variable domain (TCRVα) or fragment thereof with a third interface, and a TCR beta chain variable domain (TCRVβ) or fragment thereof with a fourth interface, wherein at least a portion of each of the TCRVα and TCRVβ together form a variable portion with binding specificity to a first target antigen;
(b) introducing one or more amino acid substitutions into the first and second interface and/or into the third and fourth interface, wherein the one or more amino acid substitutions modify the first and the second interface and/or the third and fourth interface such that the interfaces are bioorthogonal to each other (e.g., such that the bioorthogonal interfaces selectively bind to each other, e.g., selectively bind to each other as compared to unmodified interfaces), thereby enhancing the stability of the synthetic TCR molecule;
thereby producing a synthetic TCR molecule (e.g., the synthetic TCR molecule of any one of claims 1-47), wherein the synthetic TCR molecule has enhanced stability (e.g., as compared to an unmodified TCR).


SEQUENCE LISTING (88 sequences)

SEQ ID NO: 1 - protein, 96 aa, Homo sapiens
PYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSC

SEQ ID NO: 2 - protein, 131 aa, Homo sapiens
EDLKNVFPPEVAVFEPSKAEISRTQKATLVCLATGFYPPHVELSWWVNGKEVHDGVCTDPQPLKEQPALNDSRYALSSRLRVSATFWQDPRNHFRCQVQFYGLSENDEWTQDRAKPVTQIVSAEAWGRADC

SEQ ID NO: 3 - protein, 96 aa, synthetic construct
PYIQNPDPAVYQLRDSKSSDKFVCLFTDFDSQINVSQSKDSDVYITDKCVLDMRSMDFKSNRAVAWSNKSDFTCANAFNNSIIPEDTFFPSPESSC

SEQ ID NO: 4 - protein, 131 aa, synthetic construct
EDLKNVFPPEVAVFEPSKAEISRTQKATLVCLATGFYPPHVELSWWVNGKEVHDGVCTDPQPLKEQPALNDSRYALSSSLRVSATFWQDPRNHFRCQVQFYGLSENDEWTQDRAKPVTQIVSAEAWGRADC

SEQ ID NO: 5 - protein, 96 aa, synthetic construct
PYIQNPRPAVYQLRDSKSSDKFVCLFTDFDSQINVSQSKDSDVYITDKCVLDMRSMDFKSNSAVAWSNKSDFTCANAFNNSIIPEDTKFPSPESSC

SEQ ID NO: 6 - protein, 131 aa, synthetic construct
EDLKNVFPPEVAVFEPSKAEISETQKATLVCLATGFYPPHVELSWWVNGKEVHDGVCTDPQPLKEQPALNDSRYALSSRLRVSATFWQDPRNHFRCQVQFYGLSENDEWTQDRAKPVTQIVSAEAWGRADC

SEQ ID NO: 7 - protein, 96 aa, synthetic construct
PYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSC

SEQ ID NO: 8 - protein, 131 aa, synthetic construct
EDLKNVFPPEVAVFEPSKAEISETQKATLVCLATGFYPPHVELSWWVNGKEVHDGVCTDPQPLKEQPALNDSRYALSSSLRVSATFWQDPRNHFRCQVQFYGLSENDEWTQDRAKPVTQIVSAEAWGRADC

SEQ ID NO: 9 - protein, 96 aa, synthetic construct
PYIQNPQPAVYQLRDSKSSDKFVCLFTDFDSQINVSQSKDSDVYITDKCVLDMRSMDFKSNSAVAWSNKSDFTCANAFNNSIIPEDTKFPSPESSC

SEQ ID NO: 10 - protein, 131 aa, synthetic construct
EDLKNVFPPEVAVFEPSKAEISLTQKATLVCLATGFYPPHVELSWWVNGKEVHDGVCTDPQPLKEQPALNDSRYALSSRLRVSATFWQDPRNHFRCQVQFYGLSENDEWTQDRAKPVTQIVSAEAWGRADC

SEQ ID NO: 11 - protein, 96 aa, synthetic construct; identical to SEQ ID NO: 3

SEQ ID NO: 12 - protein, 131 aa, synthetic construct
EDLKNVFPPEVAVFEPSKAEISRTQKATLVCLATGFYPPHVELSWWVNGKEVHDGVCTDPQPLKEQPALNDSRYALSSTLRVSATFWQDPRNHFRCQVQFYGLSENDEWTQDRAKPVTQIVSAEAWGRADC

SEQ ID NO: 13 - protein, 96 aa, synthetic construct
PYIQNPDPAVYQLRDSKSSDKFVCLFTDFDSQINVSQSKDSDVYITDKCVLDMRSMDRKKNSAVAWSNKSDFTCANAFNNSIIPEDTFFPSPESSC

SEQ ID NO: 14 - protein, 131 aa, synthetic construct
EDLKNVFPPEVAVFEPSKAEISRTQEATLVCLATGFYPPHVELSWWVNGKEVHDGVCTDPQPLKEQPALNDSRYALSSRLSVSATFWQDPRNHFRCQVQFYGLSENDEWTQDRAKPVTQIVSAEAWGRADC

SEQ ID NO: 15 - protein, 96 aa, synthetic construct
PYIQNPFPAVYQLRDSKSSDKFVCLFTDFDSQINVSQSKDSDVYITDKCVLDMRSMDFKSNSAVAWSNKSDFTCANAFNNSIIPEDTWFPSPESSC

SEQ ID NO: 16 - protein, 131 aa, synthetic construct; identical to SEQ ID NO: 10

SEQ ID NO: 17 - protein, 96 aa, synthetic construct
PYIQNPDPAVYQLRDSKSSDKFVCLFTDFDSQINVSQSKDSDVYITDKCVLDMQDMDFKSNSAVAWSNKSDFTCANAFNNSIIPEDTFFPSPESSC

SEQ ID NO: 18 - protein, 131 aa, synthetic construct
EDLKNVFPPEVAVFEPSKAEISRTQKATLVCLATGFYPPHVELSWWVNGKEVHKGVCTDPQPLKEQPALNDSRYALSSRLRVSATFWQDPRNHFRCQVQFYGLSENDEWTQDRAKPVTQIVSAEAWGRADC

SEQ ID NO: 19 - protein, 96 aa, synthetic construct
PYIQNPDPAVYQLRDSKSSDKFVCLFTHFDSQINVSQSKDSDVYITDKCVLDMRSMDRKKNSAVAWSNKSDFTCANAFNNSIIPEDTFFPSPESSC

SEQ ID NO: 20 - protein, 131 aa, synthetic construct; identical to SEQ ID NO: 14

SEQ ID NO: 21 - protein, 96 aa, synthetic construct; identical to SEQ ID NO: 5

SEQ ID NO: 22 - protein, 131 aa, synthetic construct
EDLKNVFPPEVAVFEPSKAEISDTQKATLVCLATGFYPPHVELSWWVNGKEVHDGVCTDPQPLKEQPALNDSRYALSSRLRVSATFWQDPRNHFRCQVQFYGLSENDEWTQDRAKPVTQIVSAEAWGRADC

SEQ ID NO: 23 - protein, 96 aa, synthetic construct
PYIQNPQPAVYQLRDSKSSDKFVCLFTDFDSQINVSQSKDSDVYITDKCVLDMRSMDFKSNRAVAWSNKSDFTCANAFNNSIIPEDTKFPSPESSC

SEQ ID NO: 24 - protein, 131 aa, synthetic construct
EDLKNVFPPEVAVFEPSKAEISLTQKATLVCLATGFYPPHVELSWWVNGKEVHDGVCTDPQPLKEQPALNDSRYALSSTLRVSATFWQDPRNHFRCQVQFYGLSENDEWTQDRAKPVTQIVSAEAWGRADC

SEQ ID NO: 25 - protein, 97 aa, synthetic construct
PYIQNPQPAVYQLRDSKSSDKFVCLFTHFDSQINVSQSKDSDVYITDKCVLDMRSMDRKKNRAVAWSNKSDFTCCANAFNNSIIPEDTKFPSPESSC

SEQ ID NO: 26 - protein, 131 aa, synthetic construct
EDLKNVFPPEVAVFEPSKAEISLTQEATLVCLATGFYPPHVELSWWVNGKEVHDGVCTDPQPLKEQPALNDSRYALSSTLSVSATFWQDPRNHFRCQVQFYGLSENDEWTQDRAKPVTQIVSAEAWGRADC

SEQ ID NO: 27 - protein, 96 aa, synthetic construct; identical to SEQ ID NO: 7

SEQ ID NO: 28 - protein, 131 aa, synthetic construct
EDLKNVFPPEVAVFEPSKAEISDTQKATLVCLATGFYPPHVELSWWVNGKEVHDGVCTDPQPLKEQPALNDSRYALSSSLRVSATFWQDPRNHFRCQVQFYGLSENDEWTQDRAKPVTQIVSAEAWGRADC

SEQ ID NO: 29 - protein, 96 aa, synthetic construct
PYIQNPQPAVYQLRDSKSSDKFVCLFTDFDSQINVSQSKDSDVYITDKCVLDMRSMDRKKNRAVAWSNKSDFTCANAFNNSIIPEDTKFPSPESSC

SEQ ID NO: 30 - protein, 131 aa, synthetic construct; identical to SEQ ID NO: 26

SEQ ID NO: 31 - protein, 193 aa, Homo sapiens
MKQEVTQIPAALSVPEGENLVLNCSFTDSAIYNLQWFRQDPGKGLTSLLLIPFWQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFN

SEQ ID NO: 32 - protein, 243 aa, synthetic construct
NAGVTQTPKFQVLKTGQSMTLQCAQDMNHEYMSWYRQDPGMGLRLIHYSVSVGMTDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 33 - protein, 112 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVH

SEQ ID NO: 34 - protein, 111 aa, synthetic construct
GVTQTPKFQVLKTGQSMTLQCAQDMNHEYMSWYRYDPGMGLRLIHYSVAIQTTDQGEVPNGYNVSRSTIEDFPLRLLSAAPSQTSVYFCASSYVGDTGELFFGEGSRLTVL

SEQ ID NO: 35 - protein, 112 aa, synthetic construct
QEVTQIPAALSVPEGENLVLNCSFTDSAIYNLQWFRYDPGKGLTSLLLISPWQREQTSGRLNASLDKSSGRSTLYIAASQPGDSATYLCAVRPLLDGTYIPTFGRGTSLIVH

SEQ ID NO: 36 - protein, 111 aa, synthetic construct
GVTQTPKFQVLKTGQSMTLQCAQDMNHEYMSWYRKDPGMGLRLIHYSVAIQTTDQGEVPNGYNVSRSTIEDFPLRLLSAAPSQTSVYFCASSYVGDTGELFFGEGSRLTVL

SEQ ID NO: 37 - protein, 112 aa, synthetic construct; identical to SEQ ID NO: 33

SEQ ID NO: 38 - protein, 111 aa, synthetic construct
GVTQTPKFQVLKTGQSMTLQCAQDMNHEYMSWYRDDPGMGLRLIHYSVAIQTTDQGEVPNGYNVSRSTIEDFPLRLLSAAPSQTSVYFCASSYVGDTGELFFGEGSRLTVL

SEQ ID NO: 39 - protein, 112 aa, synthetic construct; identical to SEQ ID NO: 33

SEQ ID NO: 40 - protein, 111 aa, synthetic construct; identical to SEQ ID NO: 36

SEQ ID NO: 41 - protein, 112 aa, synthetic construct
QEVTQIPAALSVPEGENLVLNCSFTDSAIYNLQWFRVDPGKGLTSLLLISPWQREQTSGRLNASLDKSSGRSTLYIAASQPGDSATYLCAVRPLLDGTYIPTFGRGTSLIVH

SEQ ID NO: 42 - protein, 111 aa, synthetic construct
GVTQTPKFQVLKTGQSMTLQCAQDMNHEYMSWYRMDPGMGLRLIHYSVAIQTTDQGEVPNGYNVSRSTIEDFPLRLLSAAPSQTSVYFCASSYVGDTGELFFGEGSRLTVL

SEQ ID NO: 43 - protein, 112 aa, synthetic construct
QEVTQIPAALSVPEGENLVLNCSFTDSAIYNLQWFRQDPGKGLTSLLLISPWQREQTSGRLNASLDKSSGRSTLYIAASQPGDSATYLCAVRPLLDGTYIPTFGRGTKLIVH

SEQ ID NO: 44 - protein, 111 aa, synthetic construct
GVTQTPKFQVLKTGQSMTLQCAQDMNHEYMSWYRQDPEMGLRLIHYSVAIQTTDQGEVPNGYNVSRSTIEDFPLRLLSAAPSQTSVYFCASSYVGDTGELFFGEGSRLTVL

SEQ ID NO: 45 - protein, 112 aa, synthetic construct
QEVTQIPAALSVPEGENLVLNCSFTDSAIYNLQWFRLDPGKGLTSLLLISPWQREQTSGRLNASLDKSSGRSTLYIAASQPGDSATYLCAVRPLLDGTYIPTFGRGTSLIVH

SEQ ID NO: 46 - protein, 111 aa, synthetic construct; identical to SEQ ID NO: 42

SEQ ID NO: 47 - protein, 9 aa, synthetic construct
SLLMWITQC

SEQ ID NO: 48 - protein, 10 aa, synthetic construct
ELAGIGILTV

SEQ ID NO: 49 - protein, 193 aa, Homo sapiens; identical to SEQ ID NO: 31

SEQ ID NO: 50 - protein, 243 aa, Homo sapiens; identical to SEQ ID NO: 32

SEQ ID NO: 51 - protein, 722 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGSGGSGGHHHHHHHH

SEQ ID NO: 52 - protein, 727 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGSGGSGGHHHHHHHH

SEQ ID NO: 53 - protein, 722 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGSGGSGGHHHHHHHH

SEQ ID NO: 54 - protein, 727 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGSGGSGGHHHHHHHH

SEQ ID NO: 55 - protein, 722 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 56 - protein, 727 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 57 - protein, 722 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 58 - protein, 727 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 59 - protein, 722 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 60 - protein, 727 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 61 - protein, 722 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 62 - protein, 727 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 63 - protein, 680 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGSGGSGGHHHHHHHH

SEQ ID NO: 64 - protein, 682 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGSGGSGGHHHHHHHH

SEQ ID NO: 65 - protein, 693 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGSGGHHHHHHHH

SEQ ID NO: 66 - protein, 696 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGSGGHHHHHHHH

SEQ ID NO: 67 - protein, 680 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 68 - protein, 682 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 69 - protein, 693 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 70 - protein, 696 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 71 - protein, 680 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 72 - protein, 682 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 73 - protein, 693 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 74 - protein, 696 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSCGGGGSGGGGSGGGGSGGGGSQKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSCGGSGGSGGHHHHHHHH

SEQ ID NO: 75 - protein, 212 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 76 - protein, 214 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 77 - protein, 165 aa, synthetic construct
NQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 78 - protein, 228 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 79 - protein, 208 aa, synthetic construct
QEVTQIPAALSVPEGENLVLNCSFTDSAIYNLQWFRKDPGKGLTSLLLISPWQREQTSGRLNASLDKSSGRSTLYIAASQPGDSATYLCAVRPLLDGTYIPTFGRGTSLIVHPYIQNPRPAVYQLRDSKSSDKFVCLFTDFDSQINVSQSKDSDVYITDKCVLDMRSMDFKSNRAVAWSNKSDFTCANAFNNSIIPEDTKFPSPESSC

SEQ ID NO: 80 - protein, 242 aa, synthetic construct
GVTQTPKFQVLKTGQSMTLQCAQDMNHEYMSWYRYDPGMGLRLIHYSVAIQTTDQGEVPNGYNVSRSTIEDFPLRLLSAAPSQTSVYFCASSYVGDTGELFFGEGSRLTVLEDLKNVFPPEVAVFEPSKAEISETQKATLVCLATGFYPPHVELSWWVNGKEVHDGVCTDPQPLKEQPALNDSRYALSSSLRVSATFWQDPRNHFRCQVQFYGLSENDEWTQDRAKPVTQIVSAEAWGRADC

SEQ ID NO: 81 - protein, 204 aa, synthetic construct
QKEVEQNSGPLSVPEGAIASLNCTYSFLGSQSFFWYRQYSGKSPELIMFTYREGDKEDGRFTAQLNKASQHVSLLIRDSQPSDSATYLCAVNDGGRLTFGDGTTLTVKPYIQNPDPAVYQLRDSKSSDKFVCLFTDFDSQINVSQSKDSDVYITDKCVLDMRSMDFKSNSAVAWSNKSDFTCANAFNNSIIPEDTFFPSPESSC

SEQ ID NO: 82 - protein, 245 aa, synthetic construct
SQTIHQWPATLVQPVGSPLSLECTVEGTSNPNLYWYRQAAGRGPQLLFYWGPFGQISSEVPQNLSASRPQDRQFILSSKKLLLSDSGFYLCAWSETGLGMGGWQFGEGSRLTVLEDLKNVFPPEVAVFEPSKAEISRTQKATLVCLATGFYPPHVELSWWVNGKEVHDGVCTDPQPLKEQPALNDSRYALSSRLRVSATFWQDPRNHFRCQVQFYGLSENDEWTQDRAKPVTQIVSAEAWGRADC

SEQ ID NO: 83 - protein, 270 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGSGGSGGHHHHHHHH

SEQ ID NO: 84 - protein, 275 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGSGGSGGHHHHHHHH

SEQ ID NO: 85 - protein, 270 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGSGGSGGHHHHHHHH

SEQ ID NO: 86 - protein, 275 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGSGGSGGHHHHHHHH

SEQ ID NO: 87 - protein, 241 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGSGGHHHHHHHH

SEQ ID NO: 88 - protein, 244 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGSGGHHHHHHHH